(19) United States
(12) Patent Application Publication
(10) Pub. No.: US 2020/0056225 A1
(43) Pub. Date: Feb. 20, 2020

(54) METHOD FOR DIAGNOSING CHRONIC OBSTRUCTIVE AIRWAY DISEASE THROUGH BACTERIAL METAGENOME ANALYSIS

(71) Applicant: MD HEALTHCARE INC., Seoul (KR)

(72) Inventor: Yoon-Keun KIM, Gyeonggi-do (KR)

(21) Appl. No.: 16/488,258

(22) PCT Filed: Feb. 23, 2018
(86) PCT No.: PCT/KR2018/002290

§ 371 Date: Aug. 23, 2019

(30) Foreign Application Priority Data
Feb. 24, 2017 (KR) 10-2017-0025084
May 12, 2017 (KR) 10-2017-0059081
Feb. 23, 2018 (KR) 10-2018-0021708

(51) Int. Cl. C12Q 1/689

(52) U.S. Cl. CPC C12Q 1/689; C12Q 2600/112; C12Q 2600/158

(57) ABSTRACT

The present invention relates to a method of diagnosing chronic obstructive airway disease such as asthma, COPD, and the like through bacterial metagenomic analysis, and more particularly, to a method of diagnosing chronic obstructive pulmonary disease and asthma by analyzing an increase or decrease in content of extracellular vesicles derived from specific bacteria through bacterial metagenomic analysis using a subject-derived sample. Extracellular vesicles secreted from bacteria present in the environment are absorbed into the human body, and thus may directly affect inflammatory responses, and it is difficult to diagnose chronic obstructive airway diseases such as asthma, COPD, and the like early before symptoms occur, and thus efficient treatment therefor is difficult. Thus, according to the present invention, a risk of developing chronic obstructive airway diseases such as asthma, COPD, and the like can be predicted through metagenomic analysis of a gene present in bacteria-derived extracellular vesicles by using a human body-derived sample, and thus the onset of chronic obstructive airway diseases can be delayed or prevented through appropriate management by early diagnosis and prediction of a risk group for chronic obstructive airway diseases, and, even after chronic obstructive airway diseases such as asthma, COPD, and the like occur, early diagnosis therefor can be implemented, thereby lowering the incidence rate of chronic obstructive airway diseases and increasing therapeutic effects.

### TECHNICAL FIELD

The present invention relates to a method of diagnosing chronic obstructive airway diseases such as chronic obstructive pulmonary disease, asthma, and the like through bacterial metagenomic analysis, and more particularly, to a method of diagnosing chronic obstructive airway diseases by analyzing an increase or decrease in content of extracellular vesicles derived from specific bacteria through bacterial metagenomic analysis using a subject-derived sample.

### BACKGROUND ART

Chronic obstructive airway diseases are lung diseases characterized by respiratory difficulty due to chronic airway obstruction and may be broadly classified into asthma, which is characterized by reversible airway obstruction, and chronic obstructive pulmonary disease (COPD), which is characterized by irreversible airway obstruction. According to data of the National Statistical Office in 2012, the number of annual deaths due to respiratory diseases has been steadily increasing since 2006, and among these, death due to COPD is known to be the only cause of death which is on the rise, among the 10 leading causes of death, and the 2013 WHO report has shown that COPD-induced death is the fourth leading cause of death worldwide.

COPD is a disease in which chronic inflammation of the lungs causes the occurrence of chronic bronchitis, chronic bronchiolitis, and emphyema, resulting in irreversible airway obstruction. With regard to the causative factors of COPD, it is known that smoking or chemicals such as air pollutants and biological factors derived from viruses, bacteria, or the like are important. COPD is a disease that is pathologically characterized by neutrophilic inflammation, and this is immunologically characterized by Th17 cell-induced hypersensitivity. Meanwhile, asthma is a disease characterized by airway hypersensitivity to non-specific stimuli and chronic inflammatory responses, and is characterized by Th2 cell-induced hypersensitivity due to allergens such as protein antigens derived from house dust mites and the like and eosinophilic inflammation occurring due to this.

It is known that the number of microorganisms symbiotically living in the human body is 100 trillion which is 10 times the number of human cells, and the number of genes of microorganisms exceeds 100 times the number of human genes. A microbiota or microbiome is a microbial community that includes bacteria, archaea, and eukaryotes present in a given habitat. The intestinal microbiota is known to play a vital role in human's physiological phenomena and significantly affect human health and diseases through interactions with human cells. Bacteria coexisting in human bodies secrete nanometer-sized vesicles to exchange information about genes, proteins, and the like with other cells. The mucous membranes form a physical barrier membrane that does not allow particles with the size of 200 nm or more to pass therethrough, and thus bacteria symbiotically living in the mucous membranes are unable to pass therethrough, but bacteria-derived extracellular vesicles have a size of approximately 100 nm or less and thus relatively freely pass through the mucous membranes and are absorbed into the human body.

Metagenomics, also called environmental genomics, may be analytics for metagenomic data obtained from samples collected from the environment (Korean Patent Publication No. 2011-0073049). Recently, the bacterial composition of human microbiota has been listed using a method based on 16s ribosomal RNA (16s rRNA) base sequences, and 16s rDNA base sequences, which are genes of 16s ribosomal RNA, are analyzed using a next generation sequencing (NGS) platform. However, in the onset of chronic obstructive airway diseases, identification of causative factors of chronic obstructive airway diseases such as asthma, and COPD and the like through metagenomic analysis of bacteria-derived vesicles isolated from a human-derived substance, such as blood and the like, and a method of diagnosing a risk of developing chronic obstructive airway diseases have never been reported.

### DISCLOSURE

### Technical Problem

To diagnose a chronic obstructive airway diseases based on causative factors of COPD and asthma, the inventors of the present invention extracted DNA from bacteria-derived extracellular vesicles in blood, which is a subject-derived sample, and performed metagenomic analysis on the extracted DNA, and, as a result, identified bacteria-derived extracellular vesicles capable of acting as causative factors of chronic obstructive airway diseases such as COPD, asthma and the like, thus completing the present invention based on these findings.

Therefore, an object of the present invention is to provide a method of providing information for chronic obstructive airway diseases diagnosis through metagenomic analysis of bacteria-derived extracellular vesicles.

However, the technical goals of the present invention are not limited to the aforementioned goals, and other unmentioned technical goals will be clearly understood by those of ordinary skill in the art from the following description.

### Technical Solution

To achieve the above-described object of the present invention, there is provided a method of providing information for the diagnosis of a chronic obstructive airway disease, comprising the following processes:

(a) extracting DNA from extracellular vesicles isolated from a subject sample;

(b) performing polymerase chain reaction (PCR) on the extracted DNA using a pair of primers having SEQ ID NO: 1 and SEQ ID NO: 2; and

(c) comparing an increase or decrease in content of bacteria-derived extracellular vesicles between a normal individual-derived sample and a chronic obstructive pulmonary disease (COPD) patient-derived sample through sequencing of a product of the PCR,

comparing an increase or decrease in content of bacteria-derived extracellular vesicles between a normal individual-derived sample and an asthma patient-derived sample through sequencing of a product of the PCR, or

comparing an increase or decrease in content of bacteria-derived extracellular vesicles between an asthma patient-derived sample and a COPD patient-derived sample through sequencing of a product of the PCR.

The present invention also provides a method of diagnosing chronic obstructive airway disease, comprising the following processes:

(a) extracting DNA from extracellular vesicles isolated from a subject sample;

(b) performing polymerase chain reaction (PCR) on the extracted DNA using a pair of primers having SEQ ID NO: 1 and SEQ ID NO: 2; and

(c) comparing an increase or decrease in content of bacteria-derived extracellular vesicles between a normal individual-derived sample and a chronic obstructive pulmonary disease (COPD) patient-derived sample through sequencing of a product of the PCR,

comparing an increase or decrease in content of bacteria-derived extracellular vesicles between a normal individual-derived sample and an asthma patient-derived sample through sequencing of a product of the PCR, or

comparing an increase or decrease in content of bacteria-derived extracellular vesicles between an asthma patient-derived sample and a COPD patient-derived sample through sequencing of a product of the PCR.

The present invention also provides a method of predicting a risk for chronic obstructive airway disease, comprising the following processes:

(a) extracting DNA from extracellular vesicles isolated from a subject sample;

(b) performing polymerase chain reaction (PCR) on the extracted DNA using a pair of primers having SEQ ID NO: 1 and SEQ ID NO: 2; and

(c) comparing an increase or decrease in content of bacteria-derived extracellular vesicles between a normal individual-derived sample and a chronic obstructive pulmonary disease (COPD) patient-derived sample through sequencing of a product of the PCR,

comparing an increase or decrease in content of bacteria-derived extracellular vesicles between a normal individual-derived sample and an asthma patient-derived sample through sequencing of a product of the PCR, or

comparing an increase or decrease in content of bacteria-derived extracellular vesicles between an asthma patient-derived sample and a COPD patient-derived sample through sequencing of a product of the PCR.

In one embodiment of the present invention, the subject sample may be blood.

In another embodiment of the present invention, in process (c) above, COPD may be diagnosed by comparing an increase or decrease in content of extracellular vesicles derived from bacteria belonging to the phylum Tenericutes of the subject sample with that of a normal individual-derived sample.

In another embodiment of the present invention, in process (c) above, COPD may be diagnosed by comparing an increase or decrease in content of extracellular vesicles derived from one or more bacteria selected from the group consisting of the class Mollicutes and the class Solibacteres of the subject sample with that of a normal individual-derived sample.

In another embodiment of the present invention, in process (c) above, COPD may be diagnosed by comparing an increase or decrease in content of extracellular vesicles derived from one or more bacteria selected from the group consisting of the order Stramenopiles, the order Rubrobacterales, the order Turicibacterales, the order Rhodocyclales, the order RF39, and the order Solibacterales of the subject sample with that of a normal individual-derived sample.

In another embodiment of the present invention, in process (c) above, COPD may be diagnosed by comparing an increase or decrease in content of extracellular vesicles derived from one or more bacteria selected from the group consisting of the family Rubrobacteraceae, the family Turicibacteraceae, the family Rhodocyclaceae, the family Nocardiaceae, the family Clostridiaceae, the family S24-7, the family Staphylococcaceae, and the family Gordoniaceae of the subject sample with that of a normal individual-derived sample.

In another embodiment of the present invention, in process (c) above, COPD may be diagnosed by comparing an increase or decrease in content of extracellular vesicles derived from one or more bacteria selected from the group consisting of the genus Hydrogenophilus, the genus Proteus, the genus Geobacillus, the genus Chromohalobacter, the genus Rubrobacter, the genus Megamonas, the genus Turicibacter, the genus Rhodococcus, the genus Phascolarctobacterium, the genus SMB53, the genus Desulfovibrio, the genus Jeotgalicoccus, the genus Cloacibacterium, the genus Klebsiella, the genus Escherichia, the genus Cupriavidus, the genus Adlercreutzia, the genus Clostridium, the genus Faecalibacterium, the genus Stenotrophomonas, the genus Staphylococcus, the genus Gordonia, the genus Micrococcus, the genus Coprococcus, the genus Novosphingobium, the genus Enhydrobacter, the genus Citrobacter, and the genus Brevundimonas of the subject sample with that of a normal individual-derived sample.

In another embodiment of the present invention, in process (c) above, asthma may be diagnosed by comparing an increase or decrease in content of extracellular vesicles derived from one or more bacteria selected from the group consisting of the phylum Chlorollexi, the phylum Armatimonadetes, the phylum Fusobacteria, the phylum Cyanobacteria, the phylum Planctomycetes, the phylum Thermi, the phylum Verrucomicrobia, the phylum Acidobacteria, and the phylum TM7 of the subject sample with that of a normal individual-derived sample.

In another embodiment of the present invention, in process (c) above, asthma may be diagnosed by comparing an increase or decrease in content of extracellular vesicles derived from one or more bacteria selected from the group consisting of the class Rubrobacteria, the class Fimbriimonadia, the class Cytophagia, the class Chloroplast, the class Fusobacteriia, the class Saprospirae, the class Sphingobacteriia, the class Deinococci, the class Verrucomicrobiae, the class TM7-3, the class Alphaproteobacteria, the class Flavobacteriia, the class Bacilli, and the class 4C0d-2 of the subject sample with that of a normal individual-derived sample.

In another embodiment of the present invention, in process (c) above, asthma may be diagnosed by comparing an increase or decrease in content of extracellular vesicles derived from one or more bacteria selected from the group consisting of the order Rubrobacterales, the order Stramenopiles, the order Bacillales, the order Rhodocyclales, the order Fimbriimonadales, the order Cytophagales, the order Rickettsiales, the order Alteromonadales, the order Actinomycetales, the order Streptophyta, the order Fusobacteriales, the order CW040, the order Saprospirales, the order Aeromonadales, the order Neisseriales, the order Rhizobiales, the order Pseudomonadales, the order Deinococcales, the order Xanthomonadales, the order Sphingomonadales, the order Sphingobacteriales, the order Verrucomicrobiales, the order Flavobacteriales, the order Caulobacterales, the order Enterobacteriales, the order Bifidobacteriales, and the order YS2 of the subject sample with that of a normal individual-derived sample.

In another embodiment of the present invention, in process (c) above, asthma may be diagnosed by comparing an increase or decrease in content of extracellular vesicles derived from one or more bacteria selected from the group consisting of the family Rubrobacteraceae, the family Exiguobacteraceae, the family Nocardiaceae, the family F16, the family Pseudonocardiaceae, the family Dermabacteraceae, the family Brevibacteriaceae, the family Microbacteriaceae, the family Staphylococcaceae, the family Cytophagaceae, the family Planococcaceae, the family Tissierellaceae, the family Rhodocyclaceae, the family Propionibacteriaceae, the family Fimbriimonadaceae, the family Campylobacteraceae, the family Dermacoccaceae, the family Burkholderiaceae, the family Rhizobiaceae, the family Bacillaceae, the family Corynebacteriaceae, the family mitochondria, the family Fusobacteriaceae, the family Leptotrichiaceae, the family Pseudomonadaceae, the family Bradyrhizobiaceae, the family Aeromonadaceae, the family Neisseriaceae, the family Methylobacteriaceae, the family Carnobacteriaceae, the family Xanthomonadaceae, the family Geodermatophilaceae, the family Mycobacteriaceae, the family Gordoniaceae, the family Micrococcaceae, the family Hyphomicrobiaceae, the family Moraxellaceae, the family Sphingomonadaceae, the family Actinomycetaceae, the family Deinococcaceae, the family Intrasporangiaceae, the family Flavobacteriaceae, the family Lactobacillaceae, the family Verrucomicrobiaceae, the family Nocardioidaceae, the family Sphingobacteriaceae, the family Rhodospirillaceae, the family Caulobacteraceae, the family Weeksellaceae, the family Dietziaceae, the family Aerococcaceae, the family Porphyromonadaceae, the family Veillonellaceae, the family Enterobacteriaceae, the family Barnesiellaceae, the family Rikenellaceae, the family Bacteroidaceae, and the family Bifidobacteriaceae of the subject sample with that of a normal individual-derived sample.

In another embodiment of the present invention, in process (c) above, asthma may be diagnosed by comparing an increase or decrease in content of extracellular vesicles derived from one or more bacteria selected from the group consisting of the genus Geobacillus, the genus Rubrobacter, the genus Exiguobacterium, the genus Ralstonia, the genus Sporosarcina, the genus Hydrogenophilus, the genus Rhodococcus, the genus Proteus, the genus Leptotrichia, the genus Brevibacterium, the genus Brachybacterium, the genus Staphylococcus, the genus Peptomphilus, the genus Lautropia, the genus Finegoldia, the genus Anaerococcus, the genus Sphingobacterium, the genus Propionibacterium, the genus Micrococcus, the genus Fimbriimonas, the genus Dermacoccus, the genus Campylobacter, the genus Agrobacterium, the genus Neisseria, the genus Acinetobacter, the genus Thermus, the genus Corynebacterium, the genus Fusobacterium, the genus Pseudomonas, the genus Jeotgalicoccus, the genus Dietzia, the genus Rubellimicrobium, the genus Flavobacterium, the genus Megamonas, the genus Porphyromonas, the genus Granulicatella, the genus Novosphingobium, the genus Sphingomonas, the genus Mycobacterium, the genus Methylobacterium, the genus Gordonia, the genus Burkholderia, the genus Kocuria, the genus Lactobacillus, the genus Deinococcus, the genus Kaistobacter, the genus Akkermansia, the genus Actinomyces, the genus Brevundimonas, the genus Virgibacillus, the genus Bacillus, the genus Eubacterium, the genus Rothia, the genus Chryseobacterium, the genus Faecalibacterium, the genus Roseburia, the genus Klebsiella, the genus Sutterella, the genus Paraprevotella, the genus Parabacteroides, the genus Butyricimonas, the genus Lachnobacterium, the genus Veillonella, the genus Bacteroides, the genus Lachnospira, the genus Bifidobacterium, the genus Bilophila, and the genus Enterobacter of the subject sample with that of a normal individual-derived sample.

In another embodiment of the present invention, in process (c) above, asthma and COPD may be differentially diagnosed by comparing an increase or decrease in content of extracellular vesicles derived from one or more bacteria selected from the group consisting of the phylum Bacteroidetes, the phylum Tenericutes, the phylum Thermi, the phylum TM7, the phylum Cyanobacteria, the phylum Verrucomicrobia, the phylum Fusobacteria, the phylum Acidobacteria, the phylum Planctomycetes, the phylum Armatimonadetes, and the phylum Chlorollexi between an asthma patient-derived sample and a COPD patient-derived sample.

In another embodiment of the present invention, in process (c) above, asthma and COPD may be differentially diagnosed by comparing an increase or decrease in content of extracellular vesicles derived from one or more bacteria selected from the group consisting of the class Bacteroidia, the class 4C0d-2, the class Mollicutes, the class Bacilli, the class Deinococci, the class TM7-3, the class Flavobacteriia, the class Alphaproteobacteria, the class Verrucomicrobiae, the class Fusobacteriia, the class Saprospirae, the class Sphingobacteriia, the class Chloroplast, the class Cytophagia, the class Fimbriimonadia, the class Thermomicrobia, and the class Solibacteres between an asthma patient-derived sample and a COPD patient-derived sample.

In another embodiment of the present invention, in process (c) above, asthma and COPD may be differentially diagnosed by comparing an increase or decrease in content of extracellular vesicles derived from one or more bacteria selected from the group consisting of the order YS2, the order Bifidobacteriales, the order Turicibacterales, the order Bacteroidales, the order RF39, the order Enterobacteriales, the order Rhodobacterales, the order Neisseriales, the order Gemellales, the order Deinococcales, the order Flavobacteriales, the order Xanthomonadales, the order Verrucomicrobiales, the order Sphingomonadales, the order Caulobacterales, the order Fusobacteriales, the order Saprospirales, the order Pseudomonadales, the order Sphingobacteriales, the order Rhizobiales, the order Actinomycetales, the order CW040, the order Streptophyta, the order Rickettsiales, the order Alteromonadales, the order Cytophagales, the order Aeromonadales, the order Fimbriimonadales, the order JG30-KF-CM45, the order Bacillales, and the order Solibacterales between an asthma patient-derived sample and a COPD patient-derived sample.

In another embodiment of the present invention, in process (c) above, asthma and COPD may be differentially diagnosed by comparing an increase or decrease in content of extracellular vesicles derived from one or more bacteria selected from the group consisting of the family Helicobacteraceae, the family Bacteroidaceae, the family Bifidobacteriaceae, the family Turicibacteraceae, the family Rikenellaceae, the family Odoribacteraceae, the family Clostridiaceae, the family Barnesiellaceae, the family Veillonellaceae, the family Porphyromonadaceae, the family Enterobacteriaceae, the family Christensenellaceae, the family Lactobacillaceae, the family Rhodobacteraceae, the family Nocardiaceae, the family Neisseriaceae, the family Gemellaceae, the family Carnobacteriaceae, the family Aerococcaceae, the family Weeksellaceae, the family Deinococcaceae, the family Leptotrichiaceae, the family Mycobacteriaceae, the family Dietziaceae, the family Xanthomonadaceae, the family Ps eudomonadaceae, the family Verrucomicrobiaceae, the family Methylobacteriaceae, the family Flavobacteriaceae, the family Actinomycetaceae, the family Burkholderiaceae, the family Nocardioidaceae, the family Caulobacteraceae, the family Sphingomonadaceae, the family Corynebacteriaceae, the family Tissierellaceae, the family Chitinophagaceae, the family mitochondria, the family Sphingobacteriaceae, the family Fusobacteriaceae, the family Moraxellaceae, the family Micrococcaceae, the family Geodermatophilaceae, the family Dermacoccaceae, the family Intrasporangiaceae, the family Dermabacteraceae, the family Propionibacteriaceae, the family Rhodospirillaceae, the family Bradyrhizobiaceae, the family Campylobacteraceae, the family Brevibacteriaceae, the family Microbacteriaceae, the family Cellulomonadaceae, the family Gordoniaceae, the family Bacillaceae, the family Planococcaceae, the family Rhizobiaceae, the family Aeromonadaceae, the family Fimbriimonadaceae, the family Cytophagaceae, the family F16, the family Staphylococcaceae, the family Exiguobacteraceae, and the family Alteromonadaceae between an asthma patient-derived sample and a COPD patient-derived sample.

In another embodiment of the present invention, in process (c) above, asthma and COPD may be differentially diagnosed by comparing an increase or decrease in content of extracellular vesicles derived from one or more bacteria selected from the group consisting of the genus Enterobacter, the genus Trabulsiella, the genus Phascolarctobacterium, the genus Klebsiella, the genus Bifidobacterium, the genus Bacteroides, the genus Turicibacter, the genus Sutterella, the genus Butyricimonas, the genus Parabacteroides, the genus Ruminococcus, the genus Veillonella, the genus Pediococcus, the genus Desulfovibrio, the genus SMB53, the genus Roseburia, the genus Odoribacter, the genus Dialister, the genus Escherichia, the genus Sphingobium, the genus Rothia, the genus Paracoccus, the genus Lactobacillus, the genus Rhodococcus, the genus Eubacterium, the genus Granulicatella, the genus Kaistobacter, the genus Capnocytophaga, the genus Deinococcus, the genus Mycobacterium, the genus Microbispora, the genus Methylobacterium, the genus Chryseobacterium, the genus Actinomyces, the genus Porphyromonas, the genus Kocuria, the genus Akkermansia, the genus Pseudomonas, the genus Coprococcus, the genus Peptoniphilus, the genus Neisseria, the genus Corynebacterium, the genus Anaerococcus, the genus Acinetobacter, the genus Rubellimicrobium, the genus Sphingobacterium, the genus Sphingomonas, the genus Pedobacter, the genus Finegoldia, the genus Fusobacterium, the genus Lautropia, the genus Moraxella, the genus Enhydrobacter, the genus Dermacoccus, the genus Thermus, the genus Citrobacter, the genus Bacillus, the genus Stenotrophomonas, the genus Hymenobacter, the genus Brachybacterium, the genus Propionibacterium, the genus Leptotrichia, the genus Dietzia, the genus Brevibacterium, the genus Flavobacterium, the genus Gordonia, the genus Agrobacterium, the genus Fimbriimonas, the genus Novosphingobium, the genus Lysinibacillus, the genus Brevundimonas, the genus Achromobacter, the genus Micrococcus, the genus Staphylococcus, the genus Ralstonia, the genus Exiguobacterium, and the genus Alkanindiges between an asthma patient-derived sample and a COPD patient-derived sample.

In another embodiment of the present invention, the blood may be whole blood, serum, plasma, or blood mononuclear cells.

### Advantageous Effects

Extracellular vesicles secreted from bacteria present in the environment are absorbed into the human body, and thus may directly affect inflammatory responses, and it is difficult to diagnose chronic obstructive airway diseases such as asthma, COPD, and the like early before symptoms occur, and thus efficient treatment therefor is difficult. Thus, according to the present invention, a risk of developing chronic obstructive airway diseases can be predicted through metagenomic analysis of bacteria-derived extracellular vesicles by using a human body-derived sample, and thus the onset of chronic obstructive airway diseases can be delayed or prevented through appropriate management by early diagnosis and prediction of a risk group for chronic obstructive airway diseases, and, even after chronic obstructive airway diseases occur, early diagnosis therefor can be implemented, thereby lowering the incidence rate of chronic obstructive airway diseases and increasing therapeutic effects. In addition, patients diagnosed with asthma or COPD are able to avoid exposure to causative factors predicted by metagenomic analysis, whereby the progression of diseases can be ameliorated, or recurrence thereof can be prevented.

### DESCRIPTION OF DRAWINGS

FIGS. 1A and 1B are views for evaluating the distribution pattern of extracellular vesicles (EVs) derived from bacteria in vivo. FIG. 1A illustrates images showing the distribution pattern of intestinal bacteria and EVs derived from bacteria per time (0 h, 5 min, 3 h, 6 h, and 12 h) after being orally administered to mice. FIG. 1B illustrates images showing the distribution pattern of gut bacteria and EVs derived from bacteria after being orally administered to mice and, after 12 hours, blood and various organs (heart, lung, liver, kidney, spleen, adipose tissue, and muscle) of the mice were extracted.

FIG. 2 illustrates distribution results of bacteria-derived EVs exhibiting significant diagnostic performance at a phylum level, after metagenomic analysis of bacteria-derived EVs isolated from COPD patient-derived blood and normal individual-derived blood.

FIG. 3 illustrates distribution results of bacteria-derived EVs exhibiting significant diagnostic performance at a class level, after metagenomic analysis of bacteria-derived EVs isolated from COPD patient-derived blood and normal individual-derived blood.

FIG. 4 illustrates distribution results of bacteria-derived EVs exhibiting significant diagnostic performance at an order level, after metagenomic analysis of bacteria-derived EVs isolated from COPD patient-derived blood and normal individual-derived blood.

FIG. 5 illustrates distribution results of bacteria-derived EVs exhibiting significant diagnostic performance at a family level, after metagenomic analysis of bacteria-derived EVs isolated from COPD patient-derived blood and normal individual-derived blood.

FIG. 6 illustrates distribution results of bacteria-derived EVs exhibiting significant diagnostic performance at a genus level, after metagenomic analysis of bacteria-derived EVs isolated from COPD patient-derived blood and normal individual-derived blood.

FIG. 7 illustrates distribution results of bacteria-derived EVs exhibiting significant diagnostic performance at a phylum level, after metagenomic analysis of bacteria-derived EVs isolated from asthma patient-derived blood and normal individual-derived blood.

FIG. 8 illustrates distribution results of bacteria-derived EVs exhibiting significant diagnostic performance at a class level, after metagenomic analysis of bacteria-derived EVs isolated from asthma patient-derived blood and normal individual-derived blood.

FIG. 9 illustrates distribution results of bacteria-derived EVs exhibiting significant diagnostic performance at an order level, after metagenomic analysis of bacteria-derived EVs isolated from asthma patient-derived blood and normal individual-derived blood.

FIG. 10 illustrates distribution results of bacteria-derived EVs exhibiting significant diagnostic performance at a family level, after metagenomic analysis of bacteria-derived EVs isolated from asthma patient-derived blood and normal individual-derived blood.

FIG. 11 illustrates distribution results of bacteria-derived EVs exhibiting significant diagnostic performance at a genus level, after metagenomic analysis of bacteria-derived EVs isolated from asthma patient-derived blood and normal individual-derived blood.

FIG. 12 illustrates distribution results of bacteria-derived EVs exhibiting significant diagnostic performance at a phylum level, after metagenomic analysis of bacteria-derived EVs isolated from COPD patient-derived blood and asthma patient-derived blood.

FIG. 13 illustrates distribution results of bacteria-derived EVs exhibiting significant diagnostic performance at a class level, after metagenomic analysis of bacteria-derived EVs isolated from COPD patient-derived blood and asthma patient-derived blood.

FIG. 14 illustrates distribution results of bacteria-derived EVs exhibiting significant diagnostic performance at an order level, after metagenomic analysis of bacteria-derived EVs isolated from COPD patient-derived blood and asthma patient-derived blood.

FIG. 15 illustrates distribution results of bacteria-derived EVs exhibiting significant diagnostic performance at a family level, after metagenomic analysis of bacteria-derived EVs isolated from COPD patient-derived blood and asthma patient-derived blood.

FIG. 16 illustrates distribution results of bacteria-derived EVs exhibiting significant diagnostic performance at a genus level, after metagenomic analysis of bacteria-derived EVs isolated from COPD patient-derived blood and asthma patient-derived blood.

### BEST MODE

The present invention relates to a method of diagnosing chronic obstructive airway diseases such as COPD, asthma, and the like through bacterial metagenomic analysis. The inventors of the present invention extracted genes from bacteria-derived extracellular vesicles present in subject-derived samples, performed metagenomic analysis thereon, and identified bacteria-derived extracellular vesicles capable of acting as a causative factor of COPD and asthma.

Therefore, the present invention provides a method of providing information for the diagnosis of a chronic obstructive airway disease, comprising the following processes:

(a) extracting DNA from extracellular vesicles isolated from a subject sample;

(b) performing polymerase chain reaction (PCR) on the extracted DNA using a pair of primers having SEQ ID NO: 1 and SEQ ID NO: 2; and

(c) comparing an increase or decrease in content of bacteria-derived extracellular vesicles between a normal individual-derived sample and a chronic obstructive pulmonary disease (COPD) patient-derived sample through sequencing of a product of the PCR,

comparing an increase or decrease in content of bacteria-derived extracellular vesicles between a normal individual-derived sample and an asthma patient-derived sample through sequencing of a product of the PCR, or

comparing an increase or decrease in content of bacteria-derived extracellular vesicles between an asthma patient-derived sample and a COPD patient-derived sample through sequencing of a product of the PCR.

The term “chronic obstructive airway disease” as used herein is a concept comprising asthma and COPD, but the present invention is not limited thereto.

The term “COPD” as used herein is a concept comprising chronic bronchitis, chronic bronchiolitis, and emphyema, but the present invention is not limited thereto.

The term “chronic obstructive airway disease diagnosis” as used herein refers to determining whether a patient has a risk for chronic obstructive airway disease, whether the risk for chronic obstructive airway disease is relatively high, or whether chronic obstructive airway disease has already occurred. The method of the present invention may be used to delay the onset of chronic obstructive airway disease through special and appropriate care for a specific patient, which is a patient having a high risk for chronic obstructive airway disease or prevent the onset of chronic obstructive airway disease. In addition, the method may be clinically used to determine treatment by selecting the most appropriate treatment method through early diagnosis of chronic obstructive airway disease.

The term “metagenome” as used herein refers to the total of genomes including all viruses, bacteria, fungi, and the like in isolated regions such as soil, the intestines of animals, and the like, and is mainly used as a concept of genomes that explains identification of many microorganisms at once using a sequencer to analyze non-cultured microorganisms. In particular, a metagenome does not refer to a genome of one species, but refers to a mixture of genomes, including genomes of all species of an environmental unit. This term originates from the view that, when defining one species in a process in which biology is advanced into omics, various species as well as existing one species functionally interact with each other to form a complete species. Technically, it is the subject of techniques that analyzes all DNAs and RNAs regardless of species using rapid sequencing to identify all species in one environment and verify interactions and metabolism. In the present invention, bacterial metagenomic analysis is performed using bacteria-derived extracellular vesicles isolated from, for example, serum.

In the present invention, the subject sample may be blood, and the blood may be preferably whole blood, serum, plasma, or blood mononuclear cells, but the present invention is not limited thereto.

In an embodiment of the present invention, metagenomic analysis was performed on bacteria-derived extracellular vesicles in normal individual-derived blood, asthma patient-derived blood, and COPD patient-derived blood, and bacteria-derived extracellular vesicles capable of acting as a cause of the onset of COPD and asthma were actually identified by analysis at phylum, class, order, family, and genus levels.

More particularly, in one embodiment of the present invention, as a result of performing metagenomic analysis on bacteria-derived extracellular vesicles at a phylum level, the content of extracellular vesicles derived from bacteria belonging to the phylum Tenericutes was significantly different between COPD patients and normal individuals (see Example 4).

More particularly, in one embodiment of the present invention, as a result of performing metagenomic analysis on bacteria-derived extracellular vesicles at a class level, the content of extracellular vesicles derived from bacteria belonging to the class Mollicutes and the class Solibacteres was significantly different between COPD patients and normal individuals (see Example 4).

More particularly, in one embodiment of the present invention, as a result of performing metagenomic analysis on bacteria-derived extracellular vesicles at an order level, the content of extracellular vesicles derived from bacteria belonging to the order Stramenopiles, the order Rubrobacterales, the order Turicibacterales, the order Rhodocyclales, the order RF39, and the order Solibacterales was significantly different between COPD patients and normal individuals (see Example 4).

More particularly, in one embodiment of the present invention, as a result of performing metagenomic analysis on bacteria-derived extracellular vesicles at a family level, the content of extracellular vesicles derived from bacteria belonging to the family Rubrobacteraceae, the family Turicibacteraceae, the family Rhodocyclaceae, the family Nocardiaceae, the family Clostridiaceae, the family S24-7, the family Staphylococcaceae, and the family Gordoniaceae was significantly different between COPD patients and normal individuals (see Example 4).

More particularly, in one embodiment of the present invention, as a result of performing metagenomic analysis on bacteria-derived extracellular vesicles at a genus level, the content of extracellular vesicles derived from bacteria belonging to the genus Hydrogenophilus, the genus Proteus, the genus Geobacillus, the genus Chromohalobacter, the genus Rubrobacter, the genus Megamonas, the genus Turicibacter, the genus Rhodococcus, the genus Phascolarctobacterium, the genus SMB53, the genus Desulfovibrio, the genus Jeotgalicoccus, the genus Cloacibacterium, the genus Klebsiella, the genus Escherichia, the genus Cupriavidus, the genus Adlercreutzia, the genus Clostridium, the genus Faecalibacterium, the genus Stenotrophomonas, the genus Staphylococcus, the genus Gordonia, the genus Micrococcus, the genus Coprococcus, the genus Novosphingobium, the genus Enhydrobacter, the genus Citrobacter, and the genus Brevundimonas was significantly different between COPD patients and normal individuals (see Example 4).

More particularly, in one embodiment of the present invention, as a result of performing metagenomic analysis on bacteria-derived extracellular vesicles at a phylum level, the content of extracellular vesicles derived from bacteria belonging to the phylum Chlorollexi, the phylum Armatimonadetes, the phylum Fusobacteria, the phylum Cyanobacteria, the phylum Planctomycetes, the phylum Thermi, the phylum Verrucomicrobia, the phylum Acidobacteria, and the phylum TM7 was significantly different between asthma patients and normal individuals (see Example 5).

More particularly, in one embodiment of the present invention, as a result of performing metagenomic analysis on bacteria-derived extracellular vesicles at a class level, the content of extracellular vesicles derived from bacteria belonging to the class Rubrobacteria, the class Fimbriimonadia, the class Cytophagia, the class Chloroplast, the class Fusobacteriia, the class Saprospirae, the class Sphingobacteriia, the class Deinococci, the class Verrucomicrobiae, the class TM7-3, the class Alphaproteobacteria, the class Flavobacteriia, the class Bacilli, and the class 4C0d-2 was significantly different between asthma patients and normal individuals (see Example 5).

More particularly, in one embodiment of the present invention, as a result of performing metagenomic analysis on bacteria-derived extracellular vesicles at an order level, the content of extracellular vesicles derived from bacteria belonging to the order Rubrobacterales, the order Stramenopiles, the order Bacillales, the order Rhodocyclales, the order Fimbriimonadales, the order Cytophagales, the order Rickettsiales, the order Alteromonadales, the order Actinomycetales, the order Streptophyta, the order Fusobacteriales, the order CW040, the order Saprospirales, the order Aeromonadales, the order Neisseriales, the order Rhizobiales, the order Pseudomonadales, the order Deinococcales, the order Xanthomonadales, the order Sphingomonadales, the order Sphingobacteriales, the order Verrucomicrobiales, the order Flavobacteriales, the order Caulobacterales, the order Enterobacteriales, the order Bifidobacteriales, and the order YS2 was significantly different between asthma patients and normal individuals (see Example 5).

More particularly, in one embodiment of the present invention, as a result of performing metagenomic analysis on bacteria-derived extracellular vesicles at a family level, the content of extracellular vesicles derived from bacteria belonging to the family Rubrobacteraceae, the family Exiguobacteraceae, the family Nocardiaceae, the family F16, the family Pseudonocardiaceae, the family Dermabacteraceae, the family Brevibacteriaceae, the family Microbacteriaceae, the family Staphylococcaceae, the family Cytophagaceae, the family Planococcaceae, the family Tissierellaceae, the family Rhodocyclaceae, the family Propionibacteriaceae, the family Fimbriimonadaceae, the family Campylobacteraceae, the family Dermacoccaceae, the family Burkholderiaceae, the family Rhizobiaceae, the family Bacillaceae, the family Corynebacteriaceae, the family mitochondria, the family Fusobacteriaceae, the family Leptotrichiaceae, the family Pseudomonadaceae, the family Bradyrhizobiaceae, the family Aeromonadaceae, the family Neisseriaceae, the family Methylobacteriaceae, the family Carnobacteriaceae, the family Xanthomonadaceae, the family Geodermatophilaceae, the family Mycobacteriaceae, the family Gordoniaceae, the family Micrococcaceae, the family Hyphomicrobiaceae, the family Moraxellaceae, the family Sphingomonadaceae, the family Actinomycetaceae, the family Deinococcaceae, the family Intrasporangiaceae, the family Flavobacteriaceae, the family Lactobacillaceae, the family Verrucomicrobiaceae, the family Nocardioidaceae, the family Sphingobacteriaceae, the family Rhodospirillaceae, the family Caulobacteraceae, the family Weeksellaceae, the family Dietziaceae, the family Aerococcaceae, the family Porphyromonadaceae, the family Veillonellaceae, the family Enterobacteriaceae, the family Barnesiellaceae, the family Rikenellaceae, the family Bacteroidaceae, and the family Bifidobacteriaceae was significantly different between asthma patients and normal individuals (see Example 5).

More particularly, in one embodiment of the present invention, as a result of performing metagenomic analysis on bacteria-derived extracellular vesicles at a genus level, the content of extracellular vesicles derived from bacteria belonging to the genus Geobacillus, the genus Rubrobacter, the genus Exiguobacterium, the genus Ralstonia, the genus Sporosarcina, the genus Hydrogenophilus, the genus Rhodococcus, the genus Proteus, the genus Leptotrichia, the genus Brevibacterium, the genus Brachybacterium, the genus Staphylococcus, the genus Peptomphilus, the genus Lautropia, the genus Finegoldia, the genus Anaerococcus, the genus Sphingobacterium, the genus Propionibacterium, the genus Micrococcus, the genus Fimbriimonas, the genus Dermacoccus, the genus Campylobacter, the genus Agrobacterium, the genus Neisseria, the genus Acinetobacter, the genus Thermus, the genus Corynebacterium, the genus Fusobacterium, the genus Pseudomonas, the genus Jeotgalicoccus, the genus Dietzia, the genus Rubellimicrobium, the genus Flavobacterium, the genus Megamonas, the genus Porphyromonas, the genus Granulicatella, the genus Novosphingobium, the genus Sphingomonas, the genus Mycobacterium, the genus Methylobacterium, the genus Gordonia, the genus Burkholderia, the genus Kocuria, the genus Lactobacillus, the genus Deinococcus, the genus Kaistobacter, the genus Akkermansia, the genus Actinomyces, the genus Brevundimonas, the genus Virgibacillus, the genus Bacillus, the genus Eubacterium, the genus Rothia, the genus Chryseobacterium, the genus Faecalibacterium, the genus Roseburia, the genus Klebsiella, the genus Sutterella, the genus Paraprevotella, the genus Parabacteroides, the genus Butyricimonas, the genus Lachnobacterium, the genus Veillonella, the genus Bacteroides, the genus Lachnospira, the genus Bifidobacterium, the genus Bilophila, and the genus Enterobacter was significantly different between asthma patients and normal individuals (see Example 5).

More particularly, in one embodiment of the present invention, as a result of performing metagenomic analysis on bacteria-derived extracellular vesicles at a phylum level, the content of extracellular vesicles derived from bacteria belonging to the phylum Bacteroidetes , the phylum Tenericutes, the phylum Thermi, the phylum TM7, the phylum Cyanobacteria, the phylum Verrucomicrobia, the phylum Fusobacteria, the phylum Acidobacteria, the phylum Planctomycetes, the phylum Armatimonadetes, and the phylum Chlorollexi was significantly different between asthma patients and COPD patients (see Example 6).

More particularly, in one embodiment of the present invention, as a result of performing metagenomic analysis on bacteria-derived extracellular vesicles at a class level, the content of extracellular vesicles derived from bacteria belonging to the class Bacteroidia, the class 4C0d-2, the class Mollicutes, the class Bacilli, the class Deinococci, the class TM7-3, the class Flavobacteriia, the class Alphaproteobacteria, the class Verrucomicrobiae, the class Fusobacteriia, the class Saprospirae, the class Sphingobacteriia, the class Chloroplast, the class Cytophagia, the class Fimbriimonadia, the class Thermomicrobia, and the class Solibacteres was significantly different between asthma patients and COPD patients (see Example 6).

More particularly, in one embodiment of the present invention, as a result of performing metagenomic analysis on bacteria-derived extracellular vesicles at an order level, the content of extracellular vesicles derived from bacteria belonging to the order YS2, the order Bifidobacteriales, the order Turicibacterales, the order Bacteroidales, the order RF39, the order Enterobacteriales, the order Rhodobacterales, the order Neisseriales, the order Gemellales, the order Deinococcales, the order Flavobacteriales, the order Xanthomonadales, the order Verrucomicrobiales, the order Sphingomonadales, the order Caulobacterales, the order Fusobacteriales, the order Saprospirales, the order Pseudomonadales, the order Sphingobacteriales, the order Rhizobiales, the order Actinomycetales, the order CW040, the order Streptophyta, the order Rickettsiales, the order Alteromonadales, the order Cytophagales, the order Aeromonadales, the order Fimbriimonadales, the order JG30-KF-CM45, the order Bacillales, and the order Solibacterales was significantly different between asthma patients and COPD patients (see Example 6).

More particularly, in one embodiment of the present invention, as a result of performing metagenomic analysis on bacteria-derived extracellular vesicles at a family level, the content of extracellular vesicles derived from bacteria belonging to the family Helicobacteraceae, the family Bacteroidaceae, the family Bifidobacteriaceae, the family Turicibacteraceae, the family Rikenellaceae, the family Odoribacteraceae, the family Clostridiaceae, the family Barnesiellaceae, the family Veillonellaceae, the family Porphyromonadaceae, the family Enterobacteriaceae, the family Christensenellaceae, the family Lactobacillaceae, the family Rhodobacteraceae, the family Nocardiaceae, the family Neisseriaceae, the family Gemellaceae, the family Carnobacteriaceae, the family Aerococcaceae, the family Weeksellaceae, the family Deinococcaceae, the family Leptotrichiaceae, the family Mycobacteriaceae, the family Dietziaceae, the family Xanthomonadaceae, the family Pseudomonadaceae, the family Verrucomicrobiaceae, the family Methylobacteriaceae, the family Flavobacteriaceae, the family Actinomycetaceae, the family Burkholderiaceae, the family Nocardioidaceae, the family Caulobacteraceae, the family Sphingomonadaceae, the family Corynebacteriaceae, the family Tissierellaceae, the family Chitinophagaceae, the family mitochondria, the family Sphingobacteriaceae, the family Fusobacteriaceae, the family Moraxellaceae, the family Micrococcaceae, the family Geodermatophilaceae, the family Dermacoccaceae, the family Intrasporangiaceae, the family Dermabacteraceae, the family Propionibacteriaceae, the family Rhodospirillaceae, the family Bradyrhizobiaceae, the family Campylobacteraceae, the family Brevibacteriaceae, the family Microbacteriaceae, the family Cellulomonadaceae, the family Gordoniaceae, the family Bacillaceae, the family Planococcaceae, the family Rhizobiaceae, the family Aeromonadaceae, the family Fimbriimonadaceae, the family Cytophagaceae, the family F16, the family Staphylococcaceae, the family Exiguobacteraceae, and the family Alteromonadaceae was significantly different between asthma patients and COPD patients (see Example 6).

More particularly, in one embodiment of the present invention, as a result of performing metagenomic analysis on bacteria-derived extracellular vesicles at a genus level, the content of extracellular vesicles derived from bacteria belonging to the genus Enterobacter, the genus Trabulsiella, the genus Phascolarctobacterium, the genus Klebsiella, the genus Bifidobacterium, the genus Bacteroides, the genus Turicibacter, the genus Sutterella, the genus Butyricimonas, the genus Parabacteroides, the genus Ruminococcus, the genus Veillonella, the genus Pediococcus, the genus Desulfovibrio, the genus SMB53, the genus Roseburia, the genus Odoribacter, the genus Dialister, the genus Escherichia, the genus Sphingobium, the genus Rothia, the genus Paracoccus, the genus Lactobacillus, the genus Rhodococcus, the genus Eubacterium, the genus Granulicatella, the genus Kaistobacter, the genus Capnocytophaga, the genus Deinococcus, the genus Mycobacterium, the genus Microbispora, the genus Methylobacterium, the genus Chryseobacterium, the genus Actinomyces, the genus Porphyromonas, the genus Kocuria, the genus Akkermansia, the genus Pseudomonas, the genus Coprococcus, the genus Peptomphilus, the genus Neisseria, the genus Corynebacterium, the genus Anaerococcus, the genus Acinetobacter, the genus Rubellimicrobium, the genus Sphingobacterium, the genus Sphingomonas, the genus Pedobacter, the genus Finegoldia, the genus Fusobacterium, the genus Lautropia, the genus Moraxella, the genus Enhydrobacter, the genus Dermacoccus, the genus Thermus, the genus Citrobacter, the genus Bacillus, the genus Stenotrophomonas, the genus Hymenobacter, the genus Brachybacterium, the genus Propionibacterium, the genus Leptotrichia, the genus Dietzia, the genus Brevibacterium, the genus Flavobacterium, the genus Gordonia, the genus Agrobacterium, the genus Fimbriimonas, the genus Novosphingobium, the genus Lysinibacillus, the genus Brevundimonas, the genus Achromobacter, the genus Micrococcus, the genus Staphylococcus, the genus Ralstonia, the genus Exiguobacterium, and the genus Alkanindiges was significantly different between asthma patients and COPD patients (see Example 6).

From the above-described example results, it was confirmed that bacteria-derived extracellular vesicles exhibiting significant changes in content in normal individual-derived blood, asthma patient-derived blood, and COPD patient-derived blood were identified by performing metagenomic analysis on bacteria-derived extracellular vesicles isolated from blood, and COPD and asthma not only could be diagnosed, but could also be differentially diagnosed by analyzing an increase or decrease in the content of bacteria-derived extracellular vesicles at each level through metagenomic analysis.

Hereinafter, the present invention will be described with reference to exemplary examples to aid in understanding of the present invention. However, these examples are provided only for illustrative purposes and are not intended to limit the scope of the present invention.

### MODE OF THE INVENTION

### EXAMPLES

### Example 1

### Analysis of In Vivo Absorption, Distribution, and Excretion Patterns of Intestinal Bacteria and Bacteria-Derived Extracellular Vesicles

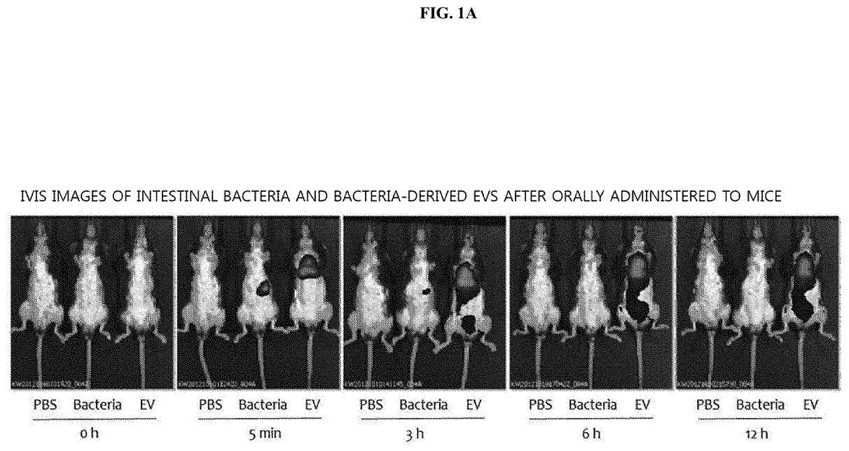

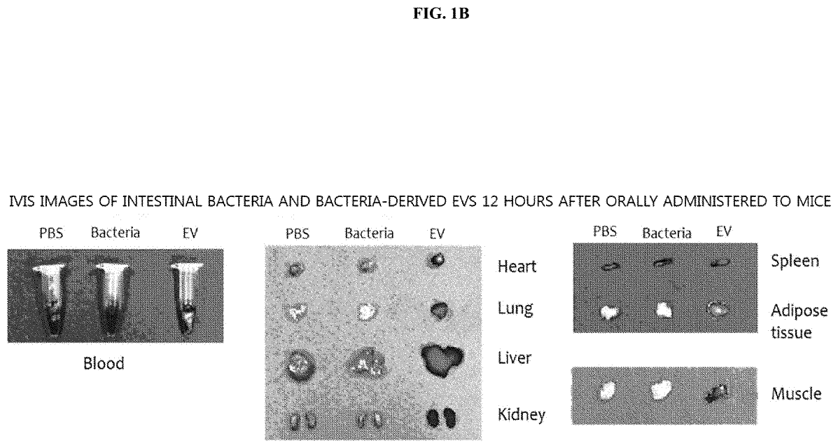

To evaluate whether intestinal bacteria and bacteria-derived extracellular vesicles are systematically absorbed through the gastrointestinal tract, an experiment was conducted using the following method. More particularly, 50 μg of each of intestinal bacteria and the bacteria-derived extracellular vesicles (EVs), labeled with fluorescence, were orally administered to the gastrointestinal tracts of mice, and fluorescence was measured at 0 h, and after 5 min, 3 h, 6 h, and 12 h. As a result of observing the entire images of mice, as illustrated in FIG. 1A, the bacteria were not systematically absorbed when administered, while the bacteria-derived EVs were systematically absorbed at 5 min after administration, and, at 3 h after administration, fluorescence was strongly observed in the bladder, from which it was confirmed that the EVs were excreted via the urinary system, and were present in the bodies up to 12 h after administration.

After intestinal bacteria and intestinal bacteria-derived extracellular vesicles were systematically absorbed, to evaluate a pattern of invasion of intestinal bacteria and the bacteria-derived EVs into various organs in the human body after being systematically absorbed, 50 μg of each of the bacteria and bacteria-derived EVs, labeled with fluorescence, were administered using the same method as that used above, and then, at 12 h after administration, blood, the heart, the lungs, the liver, the kidneys, the spleen, adipose tissue, and muscle were extracted from each mouse. As a result of observing fluorescence in the extracted tissues, as illustrated in FIG. 1B, it was confirmed that the intestinal bacteria were not absorbed into each organ, while the bacteria-derived EVs were distributed in the blood, heart, lungs, liver, kidneys, spleen, adipose tissue, and muscle.

### Example 2

### Vesicle Isolation and DNA Extraction from Blood

To isolate extracellular vesicles and extract DNA, from blood, first, stool and urine was added to a 10 ml tube and centrifuged at 3,500×g and 4 □ for 10 min to precipitate a suspension, and only a supernatant was collected, which was then placed in a new 10 ml tube. The collected supernatant was filtered using a 0.22 μm filter to remove bacteria and impurities, and then placed in centripreigugal filters (50 kD) and centrifuged at 1500×g and 4 □ for 15 min to discard materials with a smaller size than 50 kD, and then concentrated to 10 ml. Once again, bacteria and impurities were removed therefrom using a 0.22 μm filter, and then the resulting concentrate was subjected to ultra-high speed centrifugation at 150,000×g and 4 □ for 3 hours by using a Type 90ti rotor to remove a supernatant, and the agglomerated pellet was dissolved with phosphate-buffered saline (PBS), thereby obtaining vesicles.

100 μl of the extracellular vesicles isolated from the stool and urine according to the above-described method was boiled at 100 □ to allow the internal DNA to come out of the lipid and then cooled on ice. Next, the resulting vesicles were centrifuged at 10,000×g and 4 □ for 30 minutes to remove the remaining suspension, only the supernatant was collected, and then the amount of DNA extracted was quantified using a NanoDrop sprectrophotometer. In addition, to verify whether bacteria-derived DNA was present in the extracted DNA, PCR was performed using 16s rDNA primers shown in Table 1 below.

TABLE 1

| Primer | | Sequence | SEQ ID NO. |
|---|---|---|---|
| 16S rDNA | 16S_V3_F | 5′-TCGTCGGCAGCGTC | 1 |
| | | AGATGTGTATAAGAG | |
| | | ACAGCCTACGGGNGG | |
| | | CWGCAG-3′ | |
| | 16S_V4_R | 5′-GTCTCGTGGGCTCG | 2 |
| | | GAGATGTGTATAAGA | |
| | | GACAGGACTACHVGG | |
| | | GTATCTAATCC-3′ | |

### Example 3

### Metagenomic Analysis Using DNA Extracted from Vesicle in Blood

DNA was extracted using the same method as that used in Example 2, and then PCR was performed thereon using 16S rDNA primers shown in Table 1 to amplify DNA, followed by sequencing (Illumina MiSeq sequencer). The results were output as standard flowgram format (SFF) files, and the SFF files were converted into sequence files (.fasta) and nucleotide quality score files using GS FLX software (v2.9), and then credit rating for reads was identified, and portions with a window (20 bps) average base call accuracy of less than 99% (Phred score<20) were removed. After removing the low-quality portions, only reads having a length of 300 bps or more were used (Sickle version 1.33), and, for operational taxonomy unit (OTU) analysis, clustering was performed using UCLUST and USEARCH according to sequence similarity. In particular, clustering was performed based on sequence similarity values of 94% for genus, 90% for family, 85% for order, 80% for class, and 75% for phylum, and phylum, class, order, family, and genus levels of each OTU were classified, and bacteria with a sequence similarity of 97% or more were analyzed (QIIME) using 16S DNA sequence databases (108,453 sequences) of BLASTN and GreenGenes.

### Example 4

### COPD Diagnostic Model Based on Metagenomic Analysis of Bacteria-Derived EVs Isolated from Blood of Normal Individuals and COPD Patients

EVs were isolated from blood samples of 205 COPD patients and 231 normal individuals, the two groups matched in age and gender, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), sensitivity, and specificity, which are diagnostic performance indexes, were calculated by logistic regression analysis.

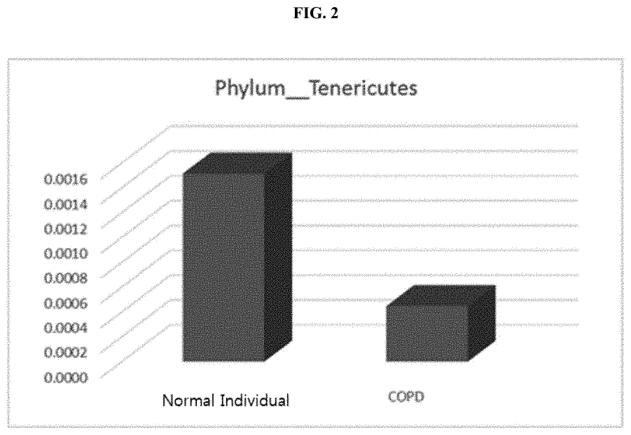

As a result of analyzing bacteria-derived extracellular vesicles in blood at a phylum level, a diagnostic model developed using bacteria belonging to the phylum Tenericutes exhibited significant diagnostic performance for COPD (see Table 2 and FIG. 2).

TABLE 2

| | normal | | | | | | | | | | | |
|---|---|---|---|---|---|---|---|---|---|---|---|---|
| | individual | | COPD | | t-test | | Training Set | | | Test Set | | |
| Taxon | Mean | SD | Mean | SD | p-value | Ratio | AUC | sensitivity | specificity | AUC | sensitivity | specificity |
| p_Tenericutes | 0.0015 | 0.0045 | 0.0004 | 0.0012 | 0.0000 | 0.30 | 0.80 | 0.88 | 0.45 | 0.76 | 0.82 | 0.47 |

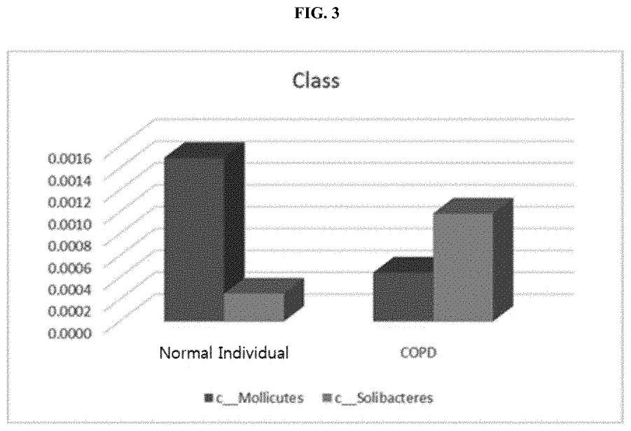

As a result of analyzing bacteria-derived extracellular vesicles in blood at a class level, a diagnostic model developed using, as a biomarker, one or more bacteria from the class Mollicutes, and the class Solibacteres exhibited significant diagnostic performance for COPD (see Table 3 and FIG. 3).

TABLE 3

| | normal | | | | | | | | | | | |
|---|---|---|---|---|---|---|---|---|---|---|---|---|
| | individual | | COPD | | t-test | | Training Set | | | Test Set | | |
| Taxon | Mean | SD | Mean | SD | p-value | Ratio | AUC | sensitivity | specificity | AUC | sensitivity | specificity |
| c_Mollicutes | 0.0015 | 0.0045 | 0.0004 | 0.0012 | 0.0000 | 0.30 | 0.80 | 0.89 | 0.41 | 0.76 | 0.83 | 0.44 |
| c_Solibacteres | 0.0003 | 0.0018 | 0.0010 | 0.0028 | 0.0008 | 3.87 | 0.78 | 0.90 | 0.19 | 0.77 | 0.87 | 0.33 |

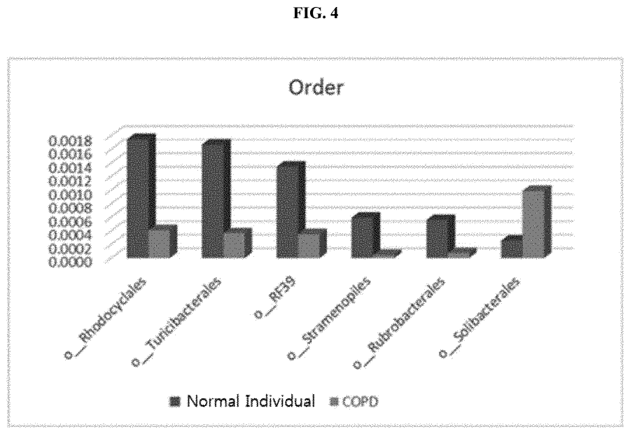

As a result of analyzing bacteria-derived extracellular vesicles in blood at an order level, a diagnostic model developed using, as a biomarker, one or more bacteria from the order Stramenopiles, the order Rubrobacterales, the order Turicibacterales, the order Rhodocyclales, the order RF39, and the order Solibacterales exhibited significant diagnostic performance for COPD (see Table 4 and FIG. 4).

TABLE 4

| | normal | | | | | | | | | | | |
|---|---|---|---|---|---|---|---|---|---|---|---|---|
| | individual | | COPD | | t-test | | | Training Set | | Test Set | | |
| Taxon | Mean | SD | Mean | SD | p-value | Ratio | AUC | sensitivity | specificity | AUC | sensitivity | specificity |
| o_Stramenopiles | 0.0006 | 0.0034 | 0.0000 | 0.0006 | 0.0010 | 0.07 | 0.79 | 0.88 | 0.33 | 0.79 | 0.87 | 0.38 |
| o_Rubrobacterales | 0.0006 | 0.0025 | 0.0001 | 0.0005 | 0.0001 | 0.13 | 0.77 | 0.87 | 0.23 | 0.74 | 0.85 | 0.29 |
| o_Turicibacterales | 0.0017 | 0.0033 | 0.0004 | 0.0014 | 0.0000 | 0.22 | 0.83 | 0.88 | 0.53 | 0.77 | 0.83 | 0.49 |
| o_Rhodocyclales | 0.0017 | 0.0075 | 0.0004 | 0.0012 | 0.0003 | 0.23 | 0.79 | 0.88 | 0.40 | 0.77 | 0.87 | 0.41 |
| o_RF39 | 0.0013 | 0.0044 | 0.0004 | 0.0011 | 0.0000 | 0.26 | 0.79 | 0.89 | 0.43 | 0.76 | 0.85 | 0.44 |
| o_Solibacterales | 0.0003 | 0.0018 | 0.0010 | 0.0028 | 0.0008 | 3.87 | 0.78 | 0.90 | 0.19 | 0.77 | 0.87 | 0.33 |

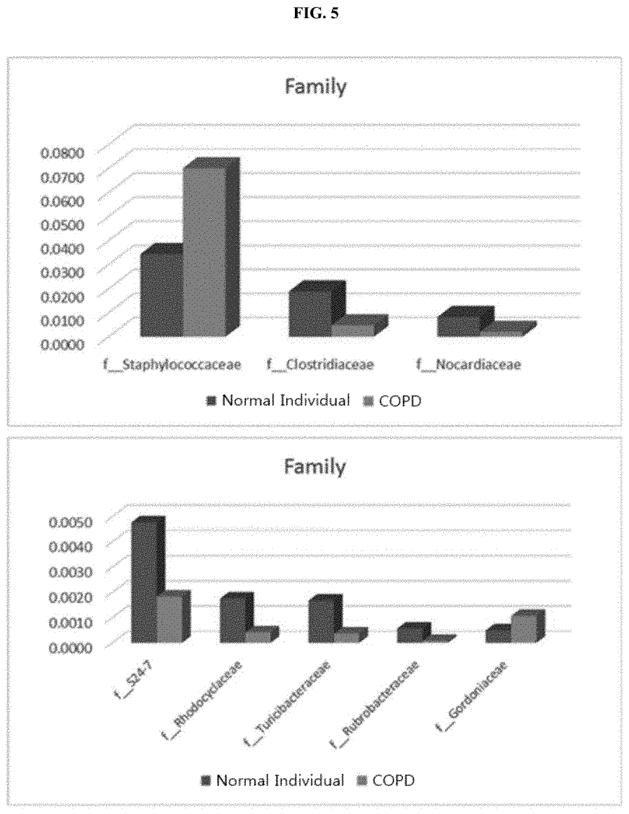

As a result of analyzing bacteria-derived extracellular vesicles in blood at a family level, a diagnostic model developed using, as a biomarker, one or more bacteria from the family Rubrobacteraceae, the family Turicibacteraceae, the family Rhodocyclaceae, the family Nocardiaceae, the family Clostridiaceae, the family S24-7, the family Staphylococcaceae, and the family Gordoniaceae exhibited significant diagnostic performance for COPD (see Table 5 and FIG. 5).

TABLE 5

| | normal | | | | | | | | | Test Set | | |
|---|---|---|---|---|---|---|---|---|---|---|---|---|
| | individual | | COPD | | t-test | | Training Set | | | | | speci- |
| Taxon | Mean | SD | Mean | SD | p-value | Ratio | AUC | sensitivity | specificity | AUC | sensitivity | ficity |
| f_Rubrobacteraceae | 0.0006 | 0.0025 | 0.0001 | 0.0005 | 0.0001 | 0.13 | 0.77 | 0.87 | 0.23 | 0.74 | 0.85 | 0.29 |
| f_Turicibacteraceae | 0.0017 | 0.0033 | 0.0004 | 0.0014 | 0.0000 | 0.22 | 0.83 | 0.88 | 0.53 | 0.77 | 0.83 | 0.49 |
| f_Rhodocyclaceae | 0.0017 | 0.0075 | 0.0004 | 0.0012 | 0.0003 | 0.23 | 0.79 | 0.88 | 0.40 | 0.77 | 0.87 | 0.42 |
| f_Nocardiaceae | 0.0082 | 0.0356 | 0.0020 | 0.0028 | 0.0003 | 0.25 | 0.77 | 0.88 | 0.24 | 0.77 | 0.87 | 0.32 |
| f_Clostridiaceae | 0.0189 | 0.0439 | 0.0048 | 0.0062 | 0.0000 | 0.25 | 0.82 | 0.87 | 0.47 | 0.80 | 0.85 | 0.58 |
| f_S24-7 | 0.0048 | 0.0125 | 0.0018 | 0.0037 | 0.0000 | 0.38 | 0.78 | 0.87 | 0.29 | 0.72 | 0.87 | 0.29 |
| f_Staphylococcaceae | 0.0344 | 0.0591 | 0.0700 | 0.0711 | 0.0000 | 2.04 | 0.82 | 0.92 | 0.33 | 0.83 | 0.90 | 0.42 |
| f_Gordoniaceae | 0.0005 | 0.0023 | 0.0011 | 0.0026 | 0.0043 | 2.17 | 0.77 | 0.88 | 0.21 | 0.76 | 0.84 | 0.29 |

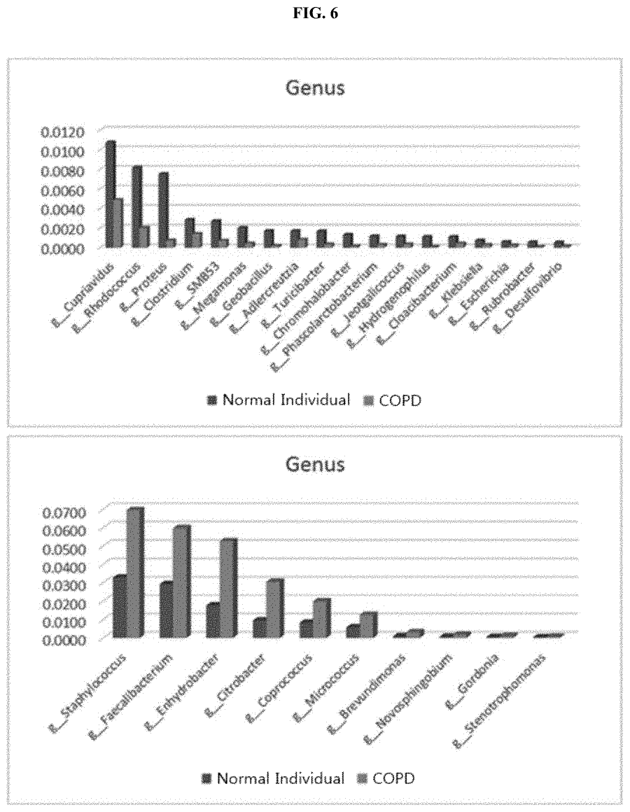

As a result of analyzing bacteria-derived extracellular vesicles in blood at a genus level, a diagnostic model developed using, as a biomarker, one or more bacteria from the genus Hydrogenophilus, the genus Proteus, the genus Geobacillus, the genus Chromohalobacter, the genus Rubrobacter, the genus Megamonas, the genus Turicibacter, the genus Rhodococcus, the genus Phascolarctobacterium, the genus SMB53, the genus Desulfovibrio, the genus Jeotgalicoccus, the genus Cloacibacterium, the genus Klebsiella, the genus Escherichia, the genus Cupriavidus, the genus Adlercreutzia, the genus Clostridium, the genus Faecalibacterium, the genus Stenotrophomonas, the genus Staphylococcus, the genus Gordonia, the genus Micrococcus, the genus Coprococcus, the genus Novosphingobium, the genus Enhydrobacter, the genus Citrobacter, and the genus Brevundimonas exhibited significant diagnostic performance for COPD (see Table 6 and FIG. 6).

TABLE 6

| | normal | | | | | | | | | Test Set | | |
|---|---|---|---|---|---|---|---|---|---|---|---|---|
| | individual | | COPD | | t-test | | Training Set | | | | | speci- |
| Taxon | Mean | SD | Mean | SD | p-value | Ratio | AUC | sensitivity | specificity | AUC | sensitivity | ficity |
| g_Hydrogenophilus | 0.0011 | 0.0070 | 0.0001 | 0.0004 | 0.0021 | 0.09 | 0.78 | 0.88 | 0.38 | 0.79 | 0.87 | 0.40 |
| g_Proteus | 0.0075 | 0.0225 | 0.0007 | 0.0024 | 0.0000 | 0.10 | 0.82 | 0.89 | 0.47 | 0.83 | 0.87 | 0.49 |
| g_Geobacillus | 0.0017 | 0.0061 | 0.0002 | 0.0009 | 0.0000 | 0.11 | 0.77 | 0.87 | 0.23 | 0.75 | 0.85 | 0.32 |
| g_Chromohalobacter | 0.0013 | 0.0073 | 0.0002 | 0.0012 | 0.0010 | 0.12 | 0.78 | 0.87 | 0.32 | 0.74 | 0.84 | 0.35 |
| g_Rubrobacter | 0.0006 | 0.0025 | 0.0001 | 0.0005 | 0.0001 | 0.13 | 0.77 | 0.87 | 0.23 | 0.74 | 0.85 | 0.29 |
| g_Megamonas | 0.0020 | 0.0094 | 0.0004 | 0.0014 | 0.0005 | 0.21 | 0.77 | 0.88 | 0.30 | 0.75 | 0.86 | 0.33 |
| g_Turicibacter | 0.0017 | 0.0033 | 0.0004 | 0.0014 | 0.0000 | 0.22 | 0.83 | 0.88 | 0.53 | 0.77 | 0.83 | 0.49 |
| g_Rhodococcus | 0.0082 | 0.0355 | 0.0020 | 0.0028 | 0.0003 | 0.25 | 0.77 | 0.88 | 0.24 | 0.77 | 0.87 | 0.32 |
| g_Phascolarctobacterium | 0.0012 | 0.0026 | 0.0003 | 0.0010 | 0.0000 | 0.26 | 0.81 | 0.88 | 0.47 | 0.74 | 0.84 | 0.44 |
| g_SMB53 | 0.0027 | 0.0076 | 0.0007 | 0.0016 | 0.0000 | 0.27 | 0.80 | 0.88 | 0.38 | 0.77 | 0.86 | 0.33 |
| g_Desulfovibrio | 0.0005 | 0.0020 | 0.0001 | 0.0006 | 0.0002 | 0.28 | 0.77 | 0.87 | 0.23 | 0.76 | 0.87 | 0.31 |
| g_Jeotgalicoccus | 0.0011 | 0.0042 | 0.0004 | 0.0016 | 0.0005 | 0.31 | 0.78 | 0.87 | 0.31 | 0.74 | 0.84 | 0.35 |
| g_Cloacibacterium | 0.0011 | 0.0037 | 0.0004 | 0.0014 | 0.0008 | 0.39 | 0.77 | 0.88 | 0.22 | 0.76 | 0.87 | 0.29 |
| g_Klebsiella | 0.0007 | 0.0017 | 0.0003 | 0.0006 | 0.0000 | 0.39 | 0.77 | 0.87 | 0.29 | 0.76 | 0.86 | 0.35 |
| g_Escherichia | 0.0006 | 0.0009 | 0.0003 | 0.0003 | 0.0000 | 0.43 | 0.82 | 0.88 | 0.44 | 0.81 | 0.87 | 0.56 |
| g_Cupriavidus | 0.0107 | 0.0237 | 0.0049 | 0.0052 | 0.0000 | 0.45 | 0.78 | 0.87 | 0.36 | 0.75 | 0.82 | 0.35 |
| g_Adlercreutzia | 0.0017 | 0.0038 | 0.0008 | 0.0014 | 0.0000 | 0.49 | 0.78 | 0.88 | 0.35 | 0.77 | 0.84 | 0.35 |
| g_Clostridium | 0.0028 | 0.0075 | 0.0014 | 0.0032 | 0.0006 | 0.50 | 0.78 | 0.87 | 0.34 | 0.75 | 0.87 | 0.36 |
| g_Faecalibacterium | 0.0292 | 0.0290 | 0.0597 | 0.0361 | 0.0000 | 2.04 | 0.86 | 0.91 | 0.53 | 0.87 | 0.91 | 0.53 |
| g_Stenotrophomonas | 0.0003 | 0.0013 | 0.0007 | 0.0016 | 0.0059 | 2.05 | 0.78 | 0.89 | 0.25 | 0.77 | 0.86 | 0.38 |
| g_Staphylocoecus | 0.0328 | 0.0588 | 0.0694 | 0.0711 | 0.0000 | 2.12 | 0.83 | 0.92 | 0.34 | 0.83 | 0.90 | 0.42 |
| g_Gordonia | 0.0005 | 0.0023 | 0.0011 | 0.0026 | 0.0043 | 2.17 | 0.77 | 0.88 | 0.21 | 0.76 | 0.84 | 0.29 |
| g_Micrococcus | 0.0057 | 0.0100 | 0.0125 | 0.0121 | 0.0000 | 2.20 | 0.82 | 0.91 | 0.37 | 0.80 | 0.85 | 0.43 |
| g_Coprococcus | 0.0083 | 0.0115 | 0.0199 | 0.0159 | 0.0000 | 2.40 | 0.86 | 0.95 | 0.54 | 0.80 | 0.92 | 0.36 |
| g_Novosphingobium | 0.0007 | 0.0027 | 0.0018 | 0.0044 | 0.0016 | 2.42 | 0.79 | 0.90 | 0.30 | 0.77 | 0.85 | 0.29 |
| g_Enhydrobacter | 0.0177 | 0.0344 | 0.0525 | 0.0478 | 0.0000 | 2.97 | 0.88 | 0.91 | 0.50 | 0.84 | 0.89 | 0.53 |
| g_Citrobacter | 0.0095 | 0.0146 | 0.0305 | 0.0214 | 0.0000 | 3.21 | 0.91 | 0.94 | 0.63 | 0.84 | 0.90 | 0.44 |
| g_Brevundimonas | 0.0009 | 0.0036 | 0.0031 | 0.0050 | 0.0000 | 3.38 | 0.80 | 0.93 | 0.31 | 0.80 | 0.91 | 0.36 |

### Example 5

### Asthma Diagnostic Model Based on Metagenomic Analysis of Bacteria-Derived EVs Isolated from Blood of Normal Individuals and Asthma Patients

Extracellular vesicles were isolated from blood samples of 219 asthma patients and 236 normal individuals, the two groups matched in age and gender, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), sensitivity, and specificity, which are diagnostic performance indexes, were calculated by logistic regression analysis.

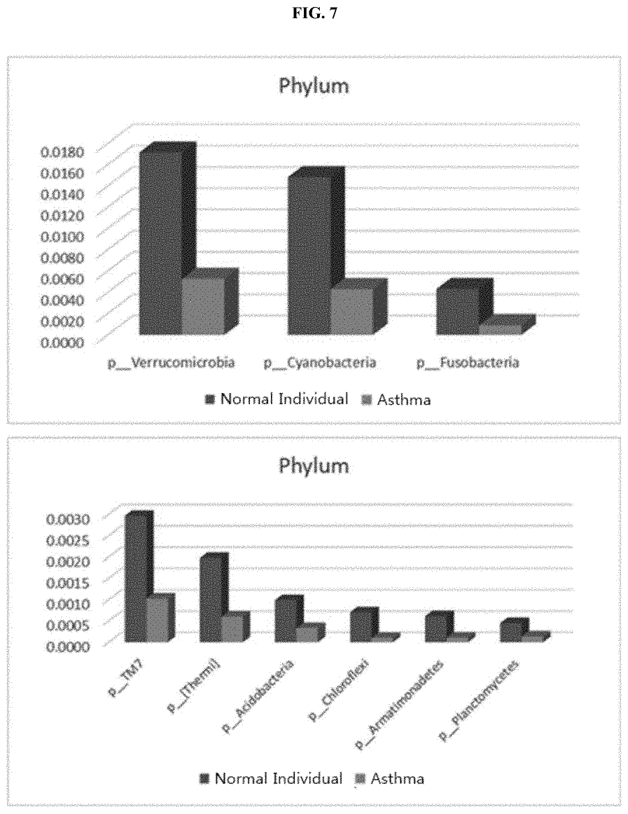

As a result of analyzing bacteria-derived extracellular vesicles in blood at a phylum level, a diagnostic model developed using, as a biomarker, one or more bacteria from the phylum Chloroflexi, the phylum Armatimonadetes, the phylum Fusobacteria, the phylum Cyanobacteria, the phylum Planctomycetes, the phylum Thermi, the phylum Verrucomicrobia, the phylum Acidobacteria, and the phylum TM7 exhibited significant diagnostic performance for asthma (see Table 7 and FIG. 7).

TABLE 7

| | normal | | | | | | | | | | | |
|---|---|---|---|---|---|---|---|---|---|---|---|---|
| | individual | | asthma | | t-test | | Training Set | | | Test Set | | |
| Taxon | Mean | SD | Mean | SD | p-value | Ratio | AUC | sensitivity | specificity | AUC | sensitivity | specificity |
| p_Chloroflexi | 0.0007 | 0.0030 | 0.0001 | 0.0003 | 0.0000 | 0.14 | 0.67 | 0.96 | 0.13 | 0.57 | 0.92 | 0.10 |
| p_Armatimonadetes | 0.0006 | 0.0024 | 0.0001 | 0.0004 | 0.0000 | 0.17 | 0.66 | 0.96 | 0.12 | 0.55 | 0.93 | 0.07 |
| p_Fusobacteria | 0.0043 | 0.0095 | 0.0009 | 0.0029 | 0.0000 | 0.21 | 0.73 | 0.90 | 0.26 | 0.68 | 0.88 | 0.24 |
| p_Cyanobacteria | 0.0148 | 0.0397 | 0.0043 | 0.0119 | 0.0000 | 0.29 | 0.72 | 0.93 | 0.18 | 0.67 | 0.89 | 0.13 |
| p_Planctomycetes | 0.0004 | 0.0018 | 0.0001 | 0.0008 | 0.0023 | 0.30 | 0.64 | 0.97 | 0.11 | 0.58 | 0.95 | 0.06 |
| p_[Thermi] | 0.0020 | 0.0047 | 0.0006 | 0.0019 | 0.0000 | 0.30 | 0.69 | 0.94 | 0.16 | 0.59 | 0.91 | 0.13 |
| p_Verrucomicrobia | 0.0172 | 0.0266 | 0.0053 | 0.0060 | 0.0000 | 0.31 | 0.78 | 0.88 | 0.38 | 0.72 | 0.82 | 0.39 |
| p_Acidobacteria | 0.0010 | 0.0033 | 0.0003 | 0.0011 | 0.0002 | 0.33 | 0.64 | 0.95 | 0.12 | 0.60 | 0.93 | 0.07 |
| p_TM7 | 0.0030 | 0.0055 | 0.0010 | 0.0029 | 0.0000 | 0.35 | 0.70 | 0.94 | 0.16 | 0.60 | 0.96 | 0.08 |

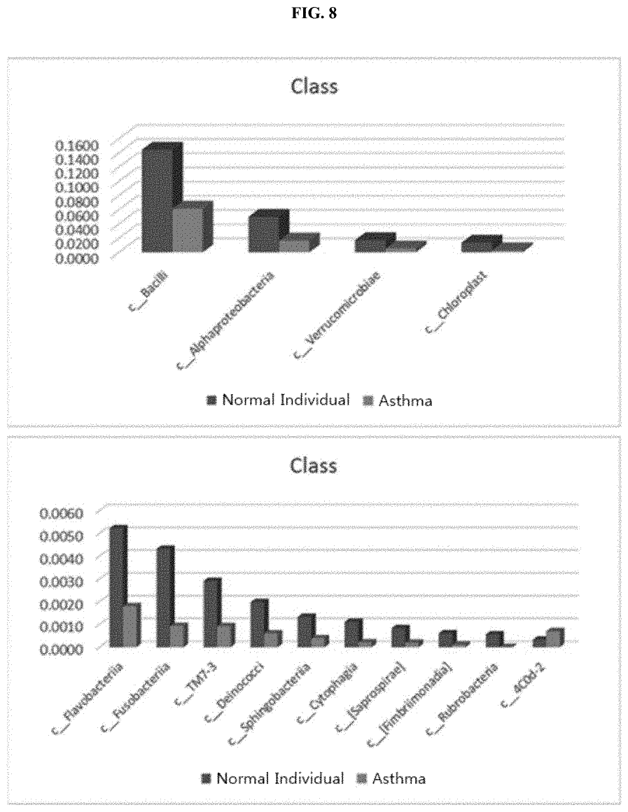

As a result of analyzing bacteria-derived extracellular vesicles in blood at a class level, a diagnostic model developed using, as a biomarker, one or more bacteria from the class Rubrobacteria, the class Fimbriimonadia, the class Cytophagia, the class Chloroplast, the class Fusobacteriia, the class Saprospirae, the class Sphingobacteriia, the class Deinococci, the class Verrucomicrobiae, the class TM7-3, the class Alphaproteobacteria, the class Flavobacteriia, the class Bacilli, and the class 4C0d-2 exhibited significant diagnostic performance for asthma (see Table 8 and FIG. 8).

TABLE 8

| | normal | | | | | | | | | Test Set | | |
|---|---|---|---|---|---|---|---|---|---|---|---|---|
| | individual | | asthma | | t-test | | Training Set | | | | | speci- |
| Taxon | Mean | SD | Mean | SD | p-value | Ratio | AUC | sensitivity | specificity | AUC | sensitivity | ficity |
| c_Rubrobacteria | 0.0006 | 0.0025 | 0.0000 | 0.0000 | 0.0000 | 0.00 | 0.67 | 0.95 | 0.16 | 0.62 | 0.93 | 0.10 |
| c_[Fimbriimonadia] | 0.0006 | 0.0024 | 0.0001 | 0.0004 | 0.0000 | 0.17 | 0.66 | 0.96 | 0.12 | 0.55 | 0.93 | 0.07 |
| c_Cytophagia | 0.0011 | 0.0036 | 0.0002 | 0.0008 | 0.0000 | 0.18 | 0.67 | 0.96 | 0.13 | 0.60 | 0.92 | 0.07 |
| c_Chloroplast | 0.0135 | 0.0394 | 0.0027 | 0.0049 | 0.0000 | 0.20 | 0.73 | 0.90 | 0.27 | 0.69 | 0.86 | 0.26 |
| c_Fusobacteriia | 0.0043 | 0.0095 | 0.0009 | 0.0029 | 0.0000 | 0.21 | 0.73 | 0.90 | 0.26 | 0.68 | 0.88 | 0.24 |
| c_[Saprospirae] | 0.0008 | 0.0028 | 0.0002 | 0.0006 | 0.0000 | 0.23 | 0.64 | 0.96 | 0.11 | 0.60 | 0.95 | 0.04 |
| c_Sphingobacteriia | 0.0013 | 0.0042 | 0.0004 | 0.0011 | 0.0000 | 0.29 | 0.65 | 0.96 | 0.10 | 0.58 | 0.92 | 0.06 |
| c_Deinococci | 0.0020 | 0.0047 | 0.0006 | 0.0019 | 0.0000 | 0.30 | 0.69 | 0.94 | 0.16 | 0.59 | 0.91 | 0.13 |
| c_Verrucomicrobiae | 0.0169 | 0.0265 | 0.0052 | 0.0060 | 0.0000 | 0.31 | 0.78 | 0.88 | 0.39 | 0.72 | 0.82 | 0.38 |
| c_TM7-3 | 0.0029 | 0.0055 | 0.0009 | 0.0027 | 0.0000 | 0.32 | 0.70 | 0.94 | 0.17 | 0.61 | 0.95 | 0.08 |
| c_Alphaproteobacteria | 0.0498 | 0.0427 | 0.0163 | 0.0323 | 0.0000 | 0.33 | 0.81 | 0.88 | 0.50 | 0.77 | 0.85 | 0.32 |
| c_Flavobacteriia | 0.0052 | 0.0076 | 0.0018 | 0.0066 | 0.0000 | 0.34 | 0.73 | 0.93 | 0.20 | 0.69 | 0.95 | 0.14 |
| c_Bacilli | 0.1441 | 0.0924 | 0.0611 | 0.0408 | 0.0000 | 0.42 | 0.87 | 0.88 | 0.65 | 0.82 | 0.85 | 0.60 |
| c_4C0d-2 | 0.0003 | 0.0012 | 0.0007 | 0.0014 | 0.0011 | 2.13 | 0.65 | 0.98 | 0.10 | 0.59 | 0.96 | 0.10 |

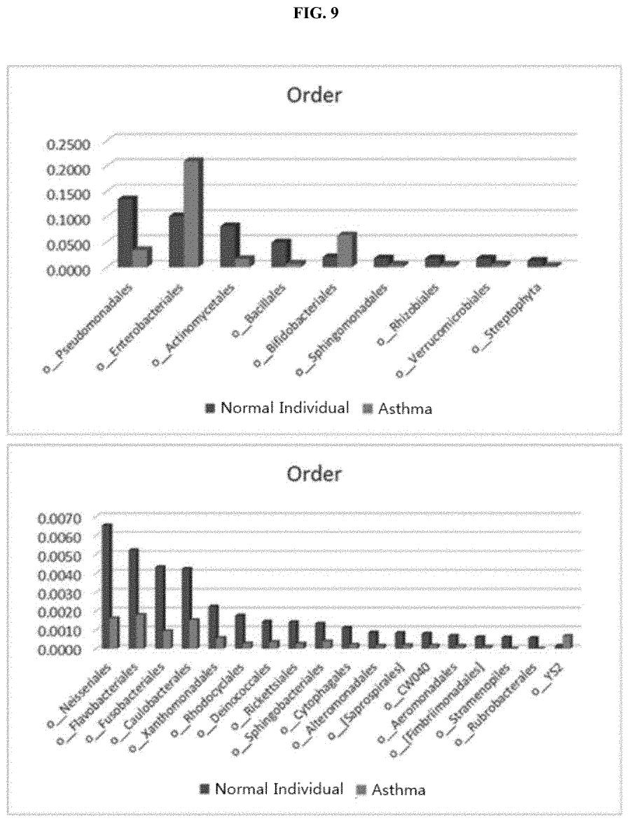

As a result of analyzing bacteria-derived extracellular vesicles in blood at an order level, a diagnostic model developed using, as a biomarker, one or more bacteria from the order Rubrobacterales, the order Stramenopiles, the order Bacillales, the order Rhodocyclales, the order Fimbriimonadales, the order Cytophagales, the order Rickettsiales, the order Alteromonadales, the order Actinomycetales, the order Streptophyta, the order Fusobacteriales, the order CW040, the order Saprospirales, the order Aeromonadales, the order Neisseriales, the order Rhizobiales, the order Pseudomonadales, the order Deinococcales, the order Xanthomonadales, the order Sphingomonadales, the order Sphingobacteriales, the order Verrucomicrobiales, the order Flavobacteriales, the order Caulobacterales, the order Enterobacteriales, the order Bifidobacteriales, and the order YS2 exhibited significant diagnostic performance for asthma (see Table 9 and FIG. 9).

TABLE 9

| | normal | | | | | | | | | Test Set | | |
|---|---|---|---|---|---|---|---|---|---|---|---|---|
| | individual | | asthma | | t-test | | Training Set | | | | | speci- |
| Taxon | Mean | SD | Mean | SD | p-value | Ratio | AUC | sensitivity | specificity | AUC | sensitivity | ficity |
| o_Rubrobacterales | 0.0006 | 0.0025 | 0.0000 | 0.0000 | 0.0000 | 0.00 | 0.67 | 0.95 | 0.16 | 0.62 | 0.93 | 0.10 |
| o_Stramenopiles | 0.0006 | 0.0034 | 0.0000 | 0.0001 | 0.0003 | 0.01 | 0.64 | 0.99 | 0.06 | 0.57 | 0.98 | 0.03 |
| o_Bacillales | 0.0484 | 0.0711 | 0.0071 | 0.0085 | 0.0000 | 0.15 | 0.88 | 0.82 | 0.73 | 0.79 | 0.77 | 0.57 |
| o_Rhodocyclales | 0.0017 | 0.0075 | 0.0003 | 0.0009 | 0.0000 | 0.16 | 0.68 | 0.97 | 0.15 | 0.63 | 0.92 | 0.10 |
| o_[Fimbriimonadales] | 0.0006 | 0.0024 | 0.0001 | 0.0004 | 0.0000 | 0.17 | 0.66 | 0.96 | 0.12 | 0.55 | 0.93 | 0.07 |
| o_Cytophagales | 0.0011 | 0.0036 | 0.0002 | 0.0008 | 0.0000 | 0.18 | 0.67 | 0.96 | 0.13 | 0.60 | 0.92 | 0.07 |
| o_Rickettsiales | 0.0014 | 0.0053 | 0.0003 | 0.0011 | 0.0000 | 0.19 | 0.67 | 0.96 | 0.13 | 0.59 | 0.94 | 0.10 |
| o_Alteromonadales | 0.0009 | 0.0023 | 0.0002 | 0.0007 | 0.0000 | 0.19 | 0.69 | 0.95 | 0.16 | 0.62 | 0.92 | 0.10 |
| o_Actinomycetales | 0.0805 | 0.0802 | 0.0160 | 0.0218 | 0.0000 | 0.20 | 0.88 | 0.84 | 0.71 | 0.80 | 0.81 | 0.53 |
| o_Streptophyta | 0.0128 | 0.0391 | 0.0027 | 0.0048 | 0.0000 | 0.21 | 0.73 | 0.90 | 0.24 | 0.69 | 0.88 | 0.22 |
| o_Fusobacteriales | 0.0043 | 0.0095 | 0.0009 | 0.0029 | 0.0000 | 0.21 | 0.73 | 0.90 | 0.26 | 0.68 | 0.88 | 0.24 |
| o_CW040 | 0.0008 | 0.0031 | 0.0002 | 0.0009 | 0.0001 | 0.22 | 0.67 | 0.97 | 0.14 | 0.57 | 0.93 | 0.10 |
| o_[Saprospirales] | 0.0008 | 0.0028 | 0.0002 | 0.0006 | 0.0000 | 0.23 | 0.64 | 0.96 | 0.11 | 0.60 | 0.95 | 0.04 |
| o_Aeromonadales | 0.0007 | 0.0028 | 0.0002 | 0.0007 | 0.0003 | 0.24 | 0.66 | 0.96 | 0.13 | 0.60 | 0.95 | 0.06 |
| o_Neisseriales | 0.0065 | 0.0178 | 0.0016 | 0.0064 | 0.0000 | 0.25 | 0.74 | 0.86 | 0.27 | 0.65 | 0.90 | 0.26 |
| o_Rhizobiales | 0.0171 | 0.0186 | 0.0043 | 0.0057 | 0.0000 | 0.25 | 0.78 | 0.84 | 0.48 | 0.75 | 0.87 | 0.33 |
| o_Pseudomonadales | 0.1336 | 0.1125 | 0.0335 | 0.0496 | 0.0000 | 0.25 | 0.85 | 0.82 | 0.65 | 0.79 | 0.80 | 0.57 |
| o_Deinococcales | 0.0014 | 0.0042 | 0.0004 | 0.0013 | 0.0000 | 0.25 | 0.68 | 0.95 | 0.16 | 0.60 | 0.91 | 0.13 |
| o_Xanthomonadales | 0.0022 | 0.0041 | 0.0006 | 0.0012 | 0.0000 | 0.26 | 0.72 | 0.93 | 0.25 | 0.66 | 0.92 | 0.21 |
| o_Sphingomonadales | 0.0173 | 0.0199 | 0.0049 | 0.0082 | 0.0000 | 0.29 | 0.78 | 0.87 | 0.44 | 0.71 | 0.88 | 0.25 |
| o_Sphingobacteriales | 0.0013 | 0.0042 | 0.0004 | 0.0011 | 0.0000 | 0.29 | 0.65 | 0.96 | 0.10 | 0.58 | 0.92 | 0.06 |
| o_Verrucomicrobiales | 0.0169 | 0.0265 | 0.0052 | 0.0060 | 0.0000 | 0.31 | 0.78 | 0.88 | 0.39 | 0.72 | 0.82 | 0.38 |
| o_Flavobacteriales | 0.0052 | 0.0076 | 0.0018 | 0.0066 | 0.0000 | 0.34 | 0.73 | 0.93 | 0.20 | 0.69 | 0.95 | 0.14 |
| o_Caulobacterales | 0.0042 | 0.0072 | 0.0015 | 0.0030 | 0.0000 | 0.36 | 0.72 | 0.90 | 0.24 | 0.59 | 0.92 | 0.18 |
| o_Enterobacteriales | 0.1006 | 0.0783 | 0.2091 | 0.0878 | 0.0000 | 2.08 | 0.84 | 0.91 | 0.54 | 0.85 | 0.93 | 0.50 |
| o_Bifidobacteriales | 0.0196 | 0.0220 | 0.0627 | 0.0335 | 0.0000 | 3.19 | 0.87 | 0.93 | 0.61 | 0.91 | 0.97 | 0.54 |
| o_YS2 | 0.0002 | 0.0006 | 0.0007 | 0.0014 | 0.0000 | 4.30 | 0.69 | 0.96 | 0.16 | 0.65 | 0.97 | 0.17 |

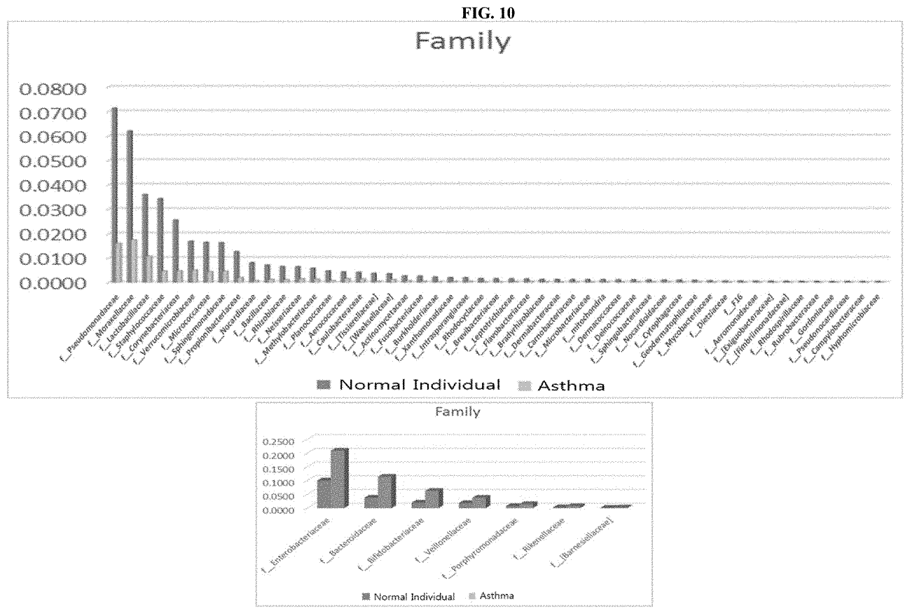

As a result of analyzing bacteria-derived extracellular vesicles in blood at a family level, a diagnostic model developed using, as a biomarker, one or more bacteria from the family Rubrobacteraceae, the family Exiguobacteraceae, the family Nocardiaceae, the family F16, the family Pseudonocardiaceae, the family Dermabacteraceae, the family Brevibacteriaceae, the family Microbacteriaceae, the family Staphylococcaceae, the family Cytophagaceae, the family Planococcaceae, the family Tissierellaceae, the family Rhodocyclaceae, the family Propionibacteriaceae, the family Fimbriimonadaceae, the family Campylobacteraceae, the family Dermacoccaceae, the family Burkholderiaceae, the family Rhizobiaceae, the family Bacillaceae, the family Corynebacteriaceae, the family mitochondria, the family Fusobacteriaceae, the family Leptotrichiaceae, the family Pseudomonadaceae, the family Bradyrhizobiaceae, the family Aeromonadaceae, the family Neisseriaceae, the family Methylobacteriaceae, the family Carnobacteriaceae, the family Xanthomonadaceae, the family Geodermatophilaceae, the family Mycobacteriaceae, the family Gordoniaceae, the family Micrococcaceae, the family Hyphomicrobiaceae, the family Moraxellaceae, the family Sphingomonadaceae, the family Actinomycetaceae, the family Deinococcaceae, the family Intrasporangiaceae, the family Flavobacteriaceae, the family Lactobacillaceae, the family Verrucomicrobiaceae, the family Nocardioidaceae, the family Sphingobacteriaceae, the family Rhodospirillaceae, the family Caulobacteraceae, the family Weeksellaceae, the family Dietziaceae, the family Aerococcaceae, the family Porphyromonadaceae, the family Veillonellaceae, the family Enterobacteriaceae, the family Barnesiellaceae, the family Rikenellaceae, the family Bacteroidaceae, and the family Bifidobacteriaceae exhibited significant diagnostic performance for asthma (see Table 10 and FIG. 10).

TABLE 10

| | normal | | | | | | | | | Test Set | | |
|---|---|---|---|---|---|---|---|---|---|---|---|---|
| | individual | | asthma | | t-test | | Training Set | | | | | speci- |
| Taxon | Mean | SD | Mean | SD | p-value | Ratio | AUC | sensitivity | specificity | AUC | sensitivity | ficity |
| f_Rubrobacteraceae | 0.0006 | 0.0025 | 0.0000 | 0.0000 | 0.0000 | 0.00 | 0.67 | 0.95 | 0.16 | 0.62 | 0.93 | 0.10 |
| f_[Exiguobacteraceae] | 0.0007 | 0.0034 | 0.0000 | 0.0002 | 0.0001 | 0.04 | 0.67 | 0.96 | 0.14 | 0.59 | 0.93 | 0.07 |
| f_Nocardiaceae | 0.0082 | 0.0356 | 0.0008 | 0.0019 | 0.0000 | 0.09 | 0.73 | 0.91 | 0.27 | 0.63 | 0.88 | 0.21 |
| f_F16 | 0.0007 | 0.0031 | 0.0001 | 0.0005 | 0.0000 | 0.09 | 0.67 | 0.96 | 0.14 | 0.60 | 0.92 | 0.10 |
| f_Pseudonocardiaceae | 0.0005 | 0.0028 | 0.0001 | 0.0002 | 0.0013 | 0.11 | 0.67 | 0.95 | 0.16 | 0.57 | 0.92 | 0.08 |
| f_Dermabacteraceae | 0.0013 | 0.0047 | 0.0002 | 0.0005 | 0.0000 | 0.12 | 0.70 | 0.92 | 0.20 | 0.62 | 0.92 | 0.10 |
| f_Brevibacteriaceae | 0.0017 | 0.0066 | 0.0002 | 0.0006 | 0.0000 | 0.12 | 0.71 | 0.92 | 0.21 | 0.64 | 0.92 | 0.15 |
| f_Microbacteriaceae | 0.0013 | 0.0079 | 0.0002 | 0.0008 | 0.0034 | 0.13 | 0.71 | 0.94 | 0.19 | 0.67 | 0.94 | 0.14 |
| f_Staphylococcaceae | 0.0344 | 0.0591 | 0.0047 | 0.0068 | 0.0000 | 0.14 | 0.85 | 0.82 | 0.61 | 0.75 | 0.80 | 0.43 |
| f_Cytophagaceae | 0.0011 | 0.0036 | 0.0002 | 0.0005 | 0.0000 | 0.15 | 0.67 | 0.96 | 0.14 | 0.58 | 0.92 | 0.07 |
| f_Planococcaceae | 0.0048 | 0.0109 | 0.0007 | 0.0013 | 0.0000 | 0.15 | 0.82 | 0.85 | 0.53 | 0.71 | 0.79 | 0.40 |
| f_[Tissierellaceae] | 0.0038 | 0.0085 | 0.0006 | 0.0016 | 0.0000 | 0.15 | 0.74 | 0.93 | 0.29 | 0.70 | 0.90 | 0.22 |
| f_Rhodocyclaceae | 0.0017 | 0.0075 | 0.0003 | 0.0009 | 0.0000 | 0.16 | 0.68 | 0.97 | 0.15 | 0.63 | 0.92 | 0.10 |
| f_Propionibacteriaceae | 0.0127 | 0.0154 | 0.0021 | 0.0031 | 0.0000 | 0.17 | 0.83 | 0.84 | 0.56 | 0.77 | 0.83 | 0.43 |
| f_[Fimbriimonadaceae] | 0.0006 | 0.0024 | 0.0001 | 0.0004 | 0.0000 | 0.17 | 0.66 | 0.96 | 0.12 | 0.55 | 0.93 | 0.07 |
| f_Campylobacteraceae | 0.0005 | 0.0016 | 0.0001 | 0.0004 | 0.0000 | 0.17 | 0.66 | 0.95 | 0.14 | 0.60 | 0.92 | 0.08 |
| f_Dermacoccaceae | 0.0012 | 0.0036 | 0.0002 | 0.0005 | 0.0000 | 0.18 | 0.68 | 0.94 | 0.18 | 0.58 | 0.90 | 0.10 |
| f_Burkholderiaceae | 0.0023 | 0.0066 | 0.0004 | 0.0010 | 0.0000 | 0.18 | 0.71 | 0.94 | 0.23 | 0.63 | 0.93 | 0.10 |
| f_Rhizobiaceae | 0.0066 | 0.0099 | 0.0012 | 0.0025 | 0.0000 | 0.18 | 0.75 | 0.86 | 0.31 | 0.71 | 0.88 | 0.25 |
| f_Bacillaceae | 0.0072 | 0.0106 | 0.0013 | 0.0020 | 0.0000 | 0.18 | 0.79 | 0.86 | 0.45 | 0.72 | 0.86 | 0.33 |
| f_Corynebacteriaceae | 0.0257 | 0.0456 | 0.0048 | 0.0055 | 0.0000 | 0.19 | 0.83 | 0.85 | 0.55 | 0.73 | 0.79 | 0.42 |
| f_mitochondria | 0.0013 | 0.0052 | 0.0003 | 0.0011 | 0.0001 | 0.21 | 0.65 | 0.96 | 0.12 | 0.58 | 0.95 | 0.08 |
| f_Fusobacteriaceae | 0.0027 | 0.0062 | 0.0006 | 0.0026 | 0.0000 | 0.21 | 0.72 | 0.92 | 0.23 | 0.66 | 0.90 | 0.15 |
| f_Leptotrichiaceae | 0.0016 | 0.0072 | 0.0004 | 0.0011 | 0.0003 | 0.22 | 0.67 | 0.96 | 0.13 | 0.59 | 0.92 | 0.04 |
| f_Pseudomonadaceae | 0.0714 | 0.0717 | 0.0160 | 0.0154 | 0.0000 | 0.22 | 0.83 | 0.82 | 0.64 | 0.75 | 0.75 | 0.56 |
| f_Bradyrhizobiaceae | 0.0014 | 0.0044 | 0.0003 | 0.0008 | 0.0000 | 0.24 | 0.67 | 0.95 | 0.13 | 0.58 | 0.92 | 0.06 |
| f_Aeromonadaceae | 0.0007 | 0.0028 | 0.0002 | 0.0007 | 0.0004 | 0.25 | 0.65 | 0.96 | 0.12 | 0.60 | 0.95 | 0.06 |
| f_Neisseriaceae | 0.0065 | 0.0178 | 0.0016 | 0.0064 | 0.0000 | 0.25 | 0.74 | 0.86 | 0.27 | 0.65 | 0.90 | 0.26 |
| f_Methylobacteriaceae | 0.0059 | 0.0101 | 0.0015 | 0.0034 | 0.0000 | 0.25 | 0.73 | 0.91 | 0.22 | 0.65 | 0.90 | 0.18 |
| f_Carnobacteriaceae | 0.0013 | 0.0031 | 0.0003 | 0.0012 | 0.0000 | 0.26 | 0.68 | 0.96 | 0.15 | 0.63 | 0.94 | 0.10 |
| f_Xanthomonadaceae | 0.0021 | 0.0041 | 0.0005 | 0.0012 | 0.0000 | 0.26 | 0.71 | 0.93 | 0.24 | 0.66 | 0.92 | 0.22 |
| f_Geodermatophilaceae | 0.0010 | 0.0033 | 0.0003 | 0.0012 | 0.0001 | 0.27 | 0.67 | 0.97 | 0.12 | 0.58 | 0.94 | 0.08 |
| f_Mycobacteriaceae | 0.0008 | 0.0028 | 0.0002 | 0.0009 | 0.0001 | 0.27 | 0.67 | 0.97 | 0.14 | 0.57 | 0.92 | 0.08 |
| f_Gordoniaceae | 0.0005 | 0.0023 | 0.0001 | 0.0005 | 0.0016 | 0.27 | 0.66 | 0.96 | 0.14 | 0.55 | 0.92 | 0.08 |
| f_Micrococcaceae | 0.0165 | 0.0213 | 0.0045 | 0.0143 | 0.0000 | 0.27 | 0.79 | 0.85 | 0.48 | 0.72 | 0.82 | 0.32 |
| f_Hyphomicrobiaceae | 0.0005 | 0.0017 | 0.0001 | 0.0006 | 0.0003 | 0.27 | 0.65 | 0.96 | 0.14 | 0.57 | 0.94 | 0.07 |
| f_Moraxellaceae | 0.0621 | 0.0670 | 0.0175 | 0.0451 | 0.0000 | 0.28 | 0.83 | 0.89 | 0.49 | 0.77 | 0.85 | 0.33 |
| f_Sphingomonadaceae | 0.0164 | 0.0190 | 0.0046 | 0.0070 | 0.0000 | 0.28 | 0.78 | 0.87 | 0.45 | 0.71 | 0.87 | 0.26 |
| f_Actinomycetaceae | 0.0027 | 0.0047 | 0.0008 | 0.0038 | 0.0000 | 0.29 | 0.76 | 0.88 | 0.35 | 0.70 | 0.88 | 0.29 |
| f_Deinococcaceae | 0.0012 | 0.0033 | 0.0003 | 0.0013 | 0.0000 | 0.29 | 0.67 | 0.96 | 0.14 | 0.59 | 0.92 | 0.08 |
| f_Intrasporangiaceae | 0.0020 | 0.0036 | 0.0006 | 0.0013 | 0.0000 | 0.30 | 0.70 | 0.93 | 0.18 | 0.64 | 0.93 | 0.11 |
| f_Flavobacteriaceae | 0.0016 | 0.0037 | 0.0005 | 0.0022 | 0.0000 | 0.30 | 0.68 | 0.94 | 0.16 | 0.60 | 0.93 | 0.13 |
| f_Lactobacillaceae | 0.0361 | 0.0559 | 0.0108 | 0.0141 | 0.0000 | 0.30 | 0.81 | 0.86 | 0.52 | 0.73 | 0.85 | 0.35 |
| f_Verrucomicrobiaceae | 0.0169 | 0.0265 | 0.0052 | 0.0060 | 0.0000 | 0.31 | 0.78 | 0.88 | 0.39 | 0.72 | 0.82 | 0.38 |
| f_Nocardioidaceae | 0.0011 | 0.0028 | 0.0003 | 0.0008 | 0.0000 | 0.31 | 0.68 | 0.94 | 0.18 | 0.59 | 0.92 | 0.13 |
| f_Sphingobacteriaceae | 0.0011 | 0.0035 | 0.0003 | 0.0010 | 0.0000 | 0.31 | 0.65 | 0.96 | 0.10 | 0.59 | 0.93 | 0.06 |
| f_Rhodospirillaceae | 0.0006 | 0.0021 | 0.0002 | 0.0011 | 0.0028 | 0.34 | 0.66 | 0.96 | 0.14 | 0.58 | 0.94 | 0.07 |
| f_Caulobacteraceae | 0.0042 | 0.0072 | 0.0015 | 0.0030 | 0.0000 | 0.36 | 0.72 | 0.90 | 0.24 | 0.59 | 0.92 | 0.18 |
| f_[Weeksellaceae] | 0.0036 | 0.0063 | 0.0013 | 0.0061 | 0.0000 | 0.36 | 0.70 | 0.96 | 0.14 | 0.66 | 0.95 | 0.07 |
| f_Dietziaceae | 0.0007 | 0.0022 | 0.0003 | 0.0008 | 0.0002 | 0.38 | 0.66 | 0.96 | 0.14 | 0.56 | 0.93 | 0.07 |
| f_Aerococcaceae | 0.0044 | 0.0069 | 0.0017 | 0.0042 | 0.0000 | 0.38 | 0.71 | 0.94 | 0.18 | 0.64 | 0.95 | 0.13 |
| f_Porphyromonadaceae | 0.0072 | 0.0163 | 0.0145 | 0.0090 | 0.0000 | 2.02 | 0.75 | 0.96 | 0.20 | 0.73 | 0.98 | 0.18 |
| f_Veillonellaceae | 0.0182 | 0.0230 | 0.0375 | 0.0288 | 0.0000 | 2.06 | 0.75 | 0.92 | 0.25 | 0.75 | 0.90 | 0.22 |
| f_Enterobacteriaceae | 0.1006 | 0.0783 | 0.2091 | 0.0878 | 0.0000 | 2.08 | 0.84 | 0.91 | 0.54 | 0.85 | 0.93 | 0.50 |
| f_[Barnesiellaceae] | 0.0010 | 0.0030 | 0.0024 | 0.0050 | 0.0001 | 2.49 | 0.68 | 0.96 | 0.13 | 0.59 | 0.94 | 0.10 |
| f_Rikenellaceae | 0.0027 | 0.0053 | 0.0070 | 0.0087 | 0.0000 | 2.60 | 0.73 | 0.93 | 0.28 | 0.68 | 0.91 | 0.19 |
| f_Bacteroidaceae | 0.0379 | 0.0377 | 0.1140 | 0.0469 | 0.0000 | 3.01 | 0.91 | 0.92 | 0.70 | 0.88 | 0.88 | 0.64 |
| f_Bifidobacteriaceae | 0.0196 | 0.0220 | 0.0627 | 0.0335 | 0.0000 | 3.19 | 0.87 | 0.93 | 0.61 | 0.91 | 0.97 | 0.54 |

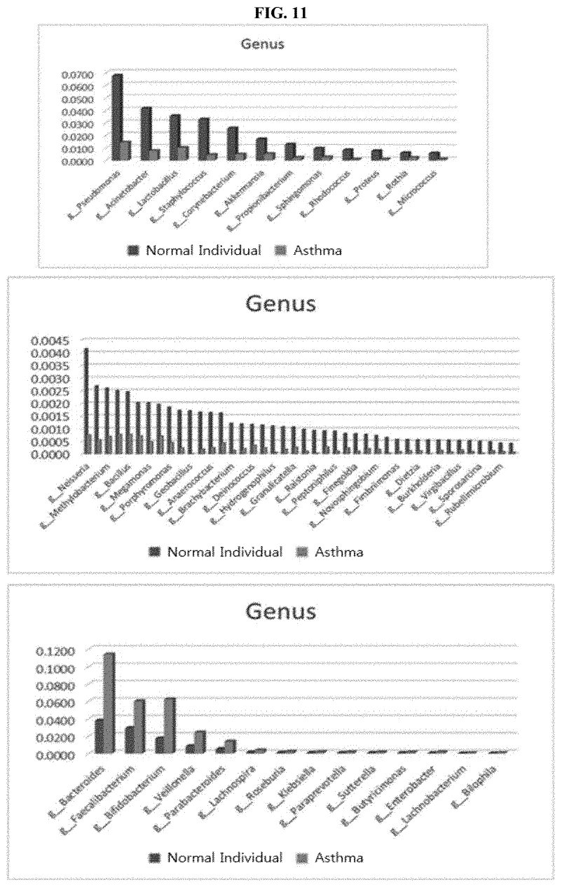

As a result of analyzing bacteria-derived extracellular vesicles in blood at a genus level, a diagnostic model developed using, as a biomarker, one or more bacteria from the genus Geobacillus, the genus Rubrobacter, the genus Exiguobacterium, the genus Ralstonia, the genus Sporosarcina, the genus Hydrogenophilus, the genus Rhodococcus, the genus Proteus, the genus Leptotrichia, the genus Brevibacterium, the genus Brachybacterium, the genus Staphylococcus, the genus Peptomphilus, the genus Lautropia, the genus Finegoldia, the genus Anaerococcus, the genus Sphingobacterium, the genus Propionibacterium, the genus Micrococcus, the genus Fimbriimonas, the genus Dermacoccus, the genus Campylobacter, the genus Agrobacterium, the genus Neisseria, the genus Acinetobacter, the genus Thermus, the genus Corynebacterium, the genus Fusobacterium, the genus Pseudomonas, the genus Jeotgalicoccus, the genus Dietzia, the genus Rubellimicrobium, the genus Flavobacterium, the genus Megamonas, the genus Porphyromonas, the genus Granulicatella, the genus Novosphingobium, the genus Sphingomonas, the genus Mycobacterium, the genus Methylobacterium, the genus Gordonia, the genus Burkholderia, the genus Kocuria, the genus Lactobacillus, the genus Deinococcus, the genus Kaistobacter, the genus Akkermansia, the genus Actinomyces, the genus Brevundimonas, the genus Virgibacillus, the genus Bacillus, the genus Eubacterium, the genus Rothia, the genus Chryseobacterium, the genus Faecalibacterium, the genus Roseburia, the genus Klebsiella, the genus Sutterella, the genus Paraprevotella, the genus Parabacteroides, the genus Butyricimonas, the genus Lachnobacterium, the genus Veillonella, the genus Bacteroides, the genus Lachnospira, the genus Bifidobacterium, the genus Bilophila, and the genus Enterobacter exhibited significant diagnostic performance for asthma (see Table 11 and FIG. 11).

TABLE 11

| | normal | | | | | | | | | | | |
|---|---|---|---|---|---|---|---|---|---|---|---|---|
| | individual | | asthma | | t-test | | Training Set | | | Test Set | | |
| Taxon | Mean | SD | Mean | SD | p-value | Ratio | AUC | sensitivity | specificity | AUC | sensitivity | specificity |
| g_Geobacillus | 0.0017 | 0.0061 | 0.0000 | 0.0000 | 0.0000 | 0.00 | 0.70 | 0.92 | 0.23 | 0.65 | 0.92 | 0.17 |
| g_Rubrobacter | 0.0006 | 0.0025 | 0.0000 | 0.0000 | 0.0000 | 0.00 | 0.67 | 0.95 | 0.16 | 0.62 | 0.93 | 0.10 |
| g_Exiguobacterium | 0.0007 | 0.0034 | 0.0000 | 0.0002 | 0.0001 | 0.04 | 0.67 | 0.97 | 0.12 | 0.59 | 0.95 | 0.04 |
| g_Ralstonia | 0.0009 | 0.0031 | 0.0000 | 0.0001 | 0.0000 | 0.04 | 0.70 | 0.94 | 0.18 | 0.61 | 0.90 | 0.13 |
| g_Sporosarcina | 0.0005 | 0.0021 | 0.0000 | 0.0002 | 0.0000 | 0.05 | 0.68 | 0.96 | 0.14 | 0.62 | 0.94 | 0.10 |
| g_Hydrogenophilus | 0.0011 | 0.0070 | 0.0001 | 0.0007 | 0.0017 | 0.06 | 0.67 | 0.97 | 0.12 | 0.62 | 0.95 | 0.06 |
| g_Rhodococcus | 0.0082 | 0.0355 | 0.0008 | 0.0019 | 0.0000 | 0.09 | 0.73 | 0.91 | 0.27 | 0.62 | 0.88 | 0.21 |
| g_Proteus | 0.0075 | 0.0225 | 0.0008 | 0.0036 | 0.0000 | 0.11 | 0.65 | 0.99 | 0.06 | 0.58 | 0.97 | 0.01 |
| g_Leptotrichia | 0.0010 | 0.0029 | 0.0001 | 0.0005 | 0.0000 | 0.12 | 0.69 | 0.94 | 0.18 | 0.59 | 0.91 | 0.13 |
| g_Brevibacterium | 0.0017 | 0.0066 | 0.0002 | 0.0006 | 0.0000 | 0.12 | 0.71 | 0.92 | 0.21 | 0.64 | 0.92 | 0.15 |
| g_Brachybacterium | 0.0012 | 0.0046 | 0.0001 | 0.0005 | 0.0000 | 0.12 | 0.70 | 0.93 | 0.19 | 0.62 | 0.92 | 0.10 |
| g_Staphylococcus | 0.0328 | 0.0588 | 0.0044 | 0.0062 | 0.0000 | 0.13 | 0.84 | 0.82 | 0.59 | 0.75 | 0.81 | 0.39 |
| g_Peptoniphilus | 0.0009 | 0.0035 | 0.0001 | 0.0005 | 0.0000 | 0.13 | 0.68 | 0.96 | 0.14 | 0.57 | 0.94 | 0.07 |
| g_Lautropia | 0.0017 | 0.0063 | 0.0002 | 0.0005 | 0.0000 | 0.14 | 0.68 | 0.95 | 0.16 | 0.60 | 0.92 | 0.07 |
| g_Finegoldia | 0.0008 | 0.0024 | 0.0001 | 0.0006 | 0.0000 | 0.14 | 0.67 | 0.96 | 0.14 | 0.62 | 0.92 | 0.08 |
| g_Anaerococcus | 0.0016 | 0.0054 | 0.0003 | 0.0013 | 0.0000 | 0.15 | 0.69 | 0.95 | 0.17 | 0.62 | 0.94 | 0.14 |
| g_Sphingobacterium | 0.0005 | 0.0028 | 0.0001 | 0.0006 | 0.0009 | 0.16 | 0.65 | 0.96 | 0.11 | 0.58 | 0.93 | 0.08 |
| g_Propionibacterium | 0.0127 | 0.0153 | 0.0021 | 0.0031 | 0.0000 | 0.17 | 0.83 | 0.84 | 0.56 | 0.77 | 0.83 | 0.43 |
| g_Micrococcus | 0.0057 | 0.0100 | 0.0010 | 0.0017 | 0.0000 | 0.17 | 0.74 | 0.87 | 0.30 | 0.70 | 0.88 | 0.28 |
| g_Fimbriimonas | 0.0006 | 0.0023 | 0.0001 | 0.0004 | 0.0000 | 0.17 | 0.66 | 0.96 | 0.13 | 0.55 | 0.95 | 0.07 |
| g_Dermacoccus | 0.0012 | 0.0036 | 0.0002 | 0.0005 | 0.0000 | 0.18 | 0.68 | 0.94 | 0.18 | 0.58 | 0.90 | 0.10 |
| g_Campylobacter | 0.0004 | 0.0015 | 0.0001 | 0.0004 | 0.0000 | 0.18 | 0.66 | 0.96 | 0.13 | 0.60 | 0.92 | 0.08 |
| g_Agrobacterium | 0.0011 | 0.0033 | 0.0002 | 0.0007 | 0.0000 | 0.18 | 0.67 | 0.96 | 0.12 | 0.58 | 0.92 | 0.04 |
| g_Neisseria | 0.0041 | 0.0107 | 0.0008 | 0.0026 | 0.0000 | 0.18 | 0.72 | 0.90 | 0.23 | 0.62 | 0.90 | 0.17 |
| g_Acinetobacter | 0.0415 | 0.0569 | 0.0076 | 0.0128 | 0.0000 | 0.18 | 0.84 | 0.84 | 0.64 | 0.79 | 0.83 | 0.49 |
| g_Thermus | 0.0005 | 0.0021 | 0.0001 | 0.0007 | 0.0001 | 0.19 | 0.67 | 0.96 | 0.12 | 0.58 | 0.92 | 0.07 |
| g_Corynebacterium | 0.0257 | 0.0456 | 0.0048 | 0.0055 | 0.0000 | 0.19 | 0.83 | 0.85 | 0.55 | 0.73 | 0.79 | 0.42 |
| g_Fusobacterium | 0.0027 | 0.0062 | 0.0006 | 0.0026 | 0.0000 | 0.21 | 0.72 | 0.92 | 0.23 | 0.66 | 0.90 | 0.15 |
| g_Pseudomonas | 0.0677 | 0.0696 | 0.0144 | 0.0145 | 0.0000 | 0.21 | 0.84 | 0.82 | 0.65 | 0.75 | 0.76 | 0.56 |
| g_Jeotgalicoccus | 0.0011 | 0.0042 | 0.0002 | 0.0013 | 0.0000 | 0.21 | 0.67 | 0.96 | 0.13 | 0.59 | 0.92 | 0.10 |
| g_Dietzia | 0.0006 | 0.0018 | 0.0001 | 0.0006 | 0.0000 | 0.22 | 0.68 | 0.95 | 0.16 | 0.60 | 0.93 | 0.08 |
| g_Rubellimicrobium | 0.0004 | 0.0016 | 0.0001 | 0.0007 | 0.0002 | 0.23 | 0.65 | 0.96 | 0.12 | 0.59 | 0.92 | 0.08 |
| g_Flavobacterium | 0.0006 | 0.0021 | 0.0001 | 0.0005 | 0.0000 | 0.23 | 0.65 | 0.96 | 0.14 | 0.57 | 0.92 | 0.08 |
| g_Megamonas | 0.0020 | 0.0094 | 0.0005 | 0.0011 | 0.0007 | 0.24 | 0.65 | 0.96 | 0.12 | 0.56 | 0.93 | 0.07 |
| g_Porphyromonas | 0.0019 | 0.0072 | 0.0005 | 0.0036 | 0.0009 | 0.25 | 0.69 | 0.94 | 0.18 | 0.62 | 0.93 | 0.14 |
| g_Granulicatella | 0.0011 | 0.0029 | 0.0003 | 0.0011 | 0.0000 | 0.26 | 0.67 | 0.95 | 0.14 | 0.62 | 0.95 | 0.08 |
| g_Novosphingobium | 0.0007 | 0.0027 | 0.0002 | 0.0009 | 0.0001 | 0.26 | 0.65 | 0.97 | 0.12 | 0.59 | 0.97 | 0.06 |
| g_Sphingomonas | 0.0094 | 0.0117 | 0.0025 | 0.0040 | 0.0000 | 0.27 | 0.77 | 0.87 | 0.38 | 0.67 | 0.86 | 0.28 |
| g_Mycobacterium | 0.0008 | 0.0028 | 0.0002 | 0.0009 | 0.0001 | 0.27 | 0.67 | 0.97 | 0.14 | 0.57 | 0.92 | 0.08 |
| g_Methylobacterium | 0.0026 | 0.0078 | 0.0007 | 0.0020 | 0.0000 | 0.27 | 0.66 | 0.96 | 0.11 | 0.62 | 0.95 | 0.06 |
| g_Gordonia | 0.0005 | 0.0023 | 0.0001 | 0.0005 | 0.0017 | 0.27 | 0.66 | 0.96 | 0.14 | 0.55 | 0.92 | 0.08 |
| g_Burkholderia | 0.0006 | 0.0019 | 0.0002 | 0.0008 | 0.0002 | 0.27 | 0.66 | 0.96 | 0.14 | 0.58 | 0.92 | 0.10 |
| g_Kocuria | 0.0016 | 0.0047 | 0.0004 | 0.0014 | 0.0000 | 0.27 | 0.68 | 0.94 | 0.13 | 0.60 | 0.93 | 0.06 |
| g_Lactobacillus | 0.0355 | 0.0560 | 0.0101 | 0.0140 | 0.0000 | 0.29 | 0.81 | 0.86 | 0.52 | 0.73 | 0.85 | 0.38 |
| g_Deinococcus | 0.0012 | 0.0033 | 0.0003 | 0.0013 | 0.0000 | 0.30 | 0.67 | 0.96 | 0.14 | 0.59 | 0.92 | 0.08 |
| g_Kaistobacter | 0.0008 | 0.0027 | 0.0002 | 0.0010 | 0.0001 | 0.30 | 0.67 | 0.96 | 0.14 | 0.55 | 0.94 | 0.04 |
| g_Akkermansia | 0.0169 | 0.0265 | 0.0052 | 0.0060 | 0.0000 | 0.31 | 0.78 | 0.88 | 0.39 | 0.72 | 0.82 | 0.38 |
| g_Actinomyces | 0.0025 | 0.0046 | 0.0008 | 0.0038 | 0.0000 | 0.31 | 0.75 | 0.89 | 0.32 | 0.69 | 0.89 | 0.26 |
| g_Brevundimonas | 0.0009 | 0.0036 | 0.0003 | 0.0008 | 0.0005 | 0.32 | 0.67 | 0.97 | 0.13 | 0.56 | 0.92 | 0.07 |
| g_Virgibacillus | 0.0005 | 0.0019 | 0.0002 | 0.0006 | 0.0002 | 0.32 | 0.65 | 0.96 | 0.12 | 0.58 | 0.92 | 0.06 |
| g_Bacillus | 0.0025 | 0.0044 | 0.0008 | 0.0016 | 0.0000 | 0.32 | 0.69 | 0.95 | 0.20 | 0.61 | 0.92 | 0.11 |
| g_[Eubacterium] | 0.0020 | 0.0043 | 0.0007 | 0.0013 | 0.0000 | 0.35 | 0.68 | 0.94 | 0.19 | 0.59 | 0.91 | 0.11 |
| g_Rothia | 0.0059 | 0.0137 | 0.0021 | 0.0139 | 0.0010 | 0.36 | 0.74 | 0.88 | 0.30 | 0.66 | 0.88 | 0.26 |
| g_Chryseobacterium | 0.0020 | 0.0045 | 0.0007 | 0.0025 | 0.0000 | 0.36 | 0.67 | 0.97 | 0.12 | 0.61 | 0.95 | 0.06 |
| g_Faecalibacterium | 0.0292 | 0.0290 | 0.0602 | 0.0236 | 0.0000 | 2.06 | 0.85 | 0.90 | 0.52 | 0.82 | 0.91 | 0.35 |
| g_Roseburia | 0.0009 | 0.0020 | 0.0021 | 0.0029 | 0.0000 | 2.24 | 0.72 | 0.94 | 0.20 | 0.64 | 0.92 | 0.14 |
| g_Klebsiella | 0.0007 | 0.0017 | 0.0018 | 0.0012 | 0.0000 | 2.48 | 0.84 | 0.94 | 0.32 | 0.76 | 0.90 | 0.28 |
| g_Sutterella | 0.0005 | 0.0017 | 0.0014 | 0.0029 | 0.0001 | 2.53 | 0.66 | 0.96 | 0.12 | 0.59 | 0.97 | 0.15 |
| g_Paraprevotella | 0.0006 | 0.0021 | 0.0014 | 0.0022 | 0.0000 | 2.54 | 0.68 | 0.98 | 0.11 | 0.61 | 0.96 | 0.11 |
| g_Parabacteroides | 0.0052 | 0.0149 | 0.0138 | 0.0086 | 0.0000 | 2.66 | 0.83 | 0.93 | 0.37 | 0.83 | 0.95 | 0.32 |
| g_Butyricimonas | 0.0004 | 0.0014 | 0.0012 | 0.0019 | 0.0000 | 2.78 | 0.72 | 0.95 | 0.18 | 0.61 | 0.92 | 0.14 |
| g_Lachnobacterium | 0.0001 | 0.0005 | 0.0004 | 0.0007 | 0.0000 | 2.86 | 0.68 | 0.95 | 0.12 | 0.62 | 0.95 | 0.17 |
| g_Veillonella | 0.0084 | 0.0152 | 0.0243 | 0.0248 | 0.0000 | 2.89 | 0.78 | 0.93 | 0.30 | 0.79 | 0.92 | 0.29 |
| g_Bacteroides | 0.0378 | 0.0377 | 0.1140 | 0.0469 | 0.0000 | 3.02 | 0.91 | 0.92 | 0.70 | 0.88 | 0.88 | 0.64 |
| g_Lachnospira | 0.0012 | 0.0029 | 0.0037 | 0.0130 | 0.0041 | 3.23 | 0.73 | 0.95 | 0.18 | 0.69 | 0.95 | 0.19 |
| g_Bifidobacterium | 0.0174 | 0.0209 | 0.0623 | 0.0336 | 0.0000 | 3.59 | 0.89 | 0.93 | 0.61 | 0.92 | 0.97 | 0.58 |
| g_Bilophila | 0.0001 | 0.0004 | 0.0005 | 0.0008 | 0.0000 | 4.25 | 0.74 | 0.94 | 0.29 | 0.69 | 0.95 | 0.21 |
| g_Enterobacter | 0.0002 | 0.0007 | 0.0016 | 0.0013 | 0.0000 | 6.56 | 0.87 | 0.94 | 0.53 | 0.88 | 0.95 | 0.54 |

### Example 6

### Model for Differential Diagnosis of COPD and Asthma Based on Metagenomic Analysis of Bacteria-Derived Extracellular Vesicles Isolated from COPD Patient-Derived Blood and Asthma Patient-Derived Blood

Extracellular vesicles were isolated from blood samples of 205 COPD patients and 219 asthma patients, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), sensitivity, and specificity, which are diagnostic performance indexes, were calculated by logistic regression analysis.

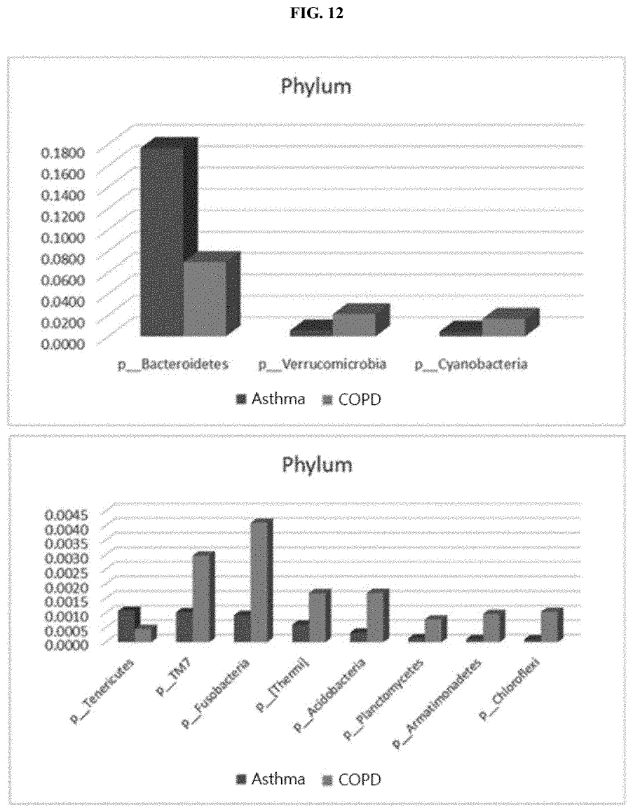

As a result of analyzing bacteria-derived extracellular vesicles in blood at a phylum level, a diagnostic model developed using, as a biomarker, one or more bacteria from the phylum Bacteroidetes, the phylum Tenericutes, the phylum Thermi, the phylum TM7, the phylum Cyanobacteria, the phylum Verrucomicrobia, the phylum Fusobacteria, the phylum Acidobacteria, the phylum Planctomycetes, the phylum Armatimonadetes, and the phylum Chloroflexi exhibited significant differential diagnostic performance for asthma and COPD (see Table 12 and FIG. 12).

TABLE 12

| | asthma | | COPD | | t-test | | Training Set | | | Test Set | | |
|---|---|---|---|---|---|---|---|---|---|---|---|---|
| Taxon | Mean | SD | Mean | SD | p-value | Ratio | AUC | sensitivity | specificity | Auc | sensitivity | specificity |
| p_Bacteroidetes | 0.1762 | 0.0499 | 0.0694 | 0.0265 | 0.0000 | 0.39 | 0.98 | 0.95 | 0.96 | 0.99 | 0.94 | 0.95 |
| p_Tenericutes | 0.0011 | 0.0018 | 0.0004 | 0.0012 | 0.0000 | 0.42 | 0.85 | 0.72 | 0.90 | 0.82 | 0.75 | 0.85 |
| p_[Thermi] | 0.0006 | 0.0019 | 0.0017 | 0.0033 | 0.0000 | 2.80 | 0.85 | 0.68 | 0.92 | 0.84 | 0.76 | 0.87 |
| p_TM7 | 0.0010 | 0.0029 | 0.0030 | 0.0044 | 0.0000 | 2.91 | 0.86 | 0.68 | 0.91 | 0.86 | 0.75 | 0.87 |
| p_Cyanobacteria | 0.0043 | 0.0119 | 0.0162 | 0.0200 | 0.0000 | 3.75 | 0.88 | 0.70 | 0.91 | 0.89 | 0.75 | 0.85 |
| p_Verrucomicrobia | 0.0053 | 0.0060 | 0.0206 | 0.0140 | 0.0000 | 3.92 | 0.93 | 0.87 | 0.84 | 0.94 | 0.90 | 0.78 |
| p_Fusobacteria | 0.0009 | 0.0029 | 0.0041 | 0.0055 | 0.0000 | 4.42 | 0.88 | 0.68 | 0.90 | 0.88 | 0.76 | 0.85 |
| p_Acidobacteria | 0.0003 | 0.0011 | 0.0017 | 0.0042 | 0.0000 | 5.13 | 0.85 | 0.67 | 0.89 | 0.86 | 0.76 | 0.85 |
| p_Planctomycetes | 0.0001 | 0.0008 | 0.0008 | 0.0031 | 0.0049 | 5.81 | 0.85 | 0.68 | 0.90 | 0.82 | 0.75 | 0.85 |
| p_Armatimonadetes | 0.0001 | 0.0004 | 0.0010 | 0.0027 | 0.0000 | 9.14 | 0.85 | 0.68 | 0.90 | 0.86 | 0.75 | 0.83 |
| p_Chloroflexi | 0.0001 | 0.0003 | 0.0010 | 0.0029 | 0.0000 | 10.56 | 0.86 | 0.68 | 0.90 | 0.85 | 0.75 | 0.85 |

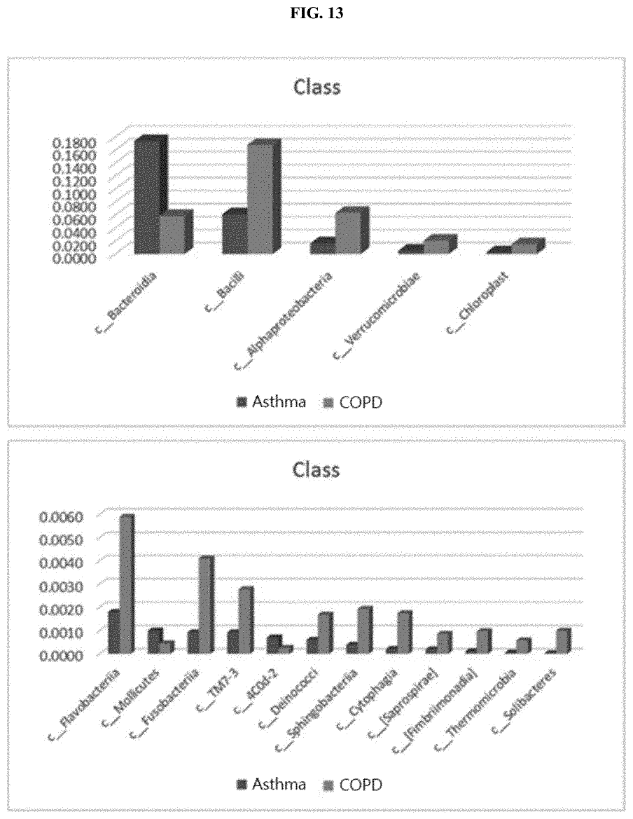

As a result of analyzing bacteria-derived extracellular vesicles in blood at a class level, a diagnostic model developed using, as a biomarker, one or more bacteria from the class Bacteroidia, the class 4C0d-2, the class Mollicutes, the class Bacilli, the class Deinococci, the class TM7-3, the class Flavobacteriia, the class Alphaproteobacteria, the class Verrucomicrobiae, the class Fusobacteriia, the class Saprospirae, the class Sphingobacteriia, the class Chloroplast, the class Cytophagia, the class Fimbriimonadia, the class Thermomicrobia, and the class Solibacteres exhibited significant differential diagnostic performance for asthma and COPD (see Table 13 and FIG. 13).

TABLE 13

| | asthma | | COPD | | t-test | | Training Set | | | Test Set | | |
|---|---|---|---|---|---|---|---|---|---|---|---|---|
| Taxon | Mean | SD | Mean | SD | p-value | Ratio | AUC | sensitivity | specificity | AUC | sensitivity | specificity |
| c_Bacteroidia | 0.1736 | 0.0514 | 0.0585 | 0.0261 | 0.0000 | 0.34 | 0.98 | 0.94 | 0.97 | 0.99 | 0.99 | 0.93 |
| c_4C0d-2 | 0.0007 | 0.0014 | 0.0003 | 0.0010 | 0.0003 | 0.36 | 0.84 | 0.64 | 0.88 | 0.82 | 0.72 | 0.87 |
| c_Mollicutes | 0.0010 | 0.0016 | 0.0004 | 0.0012 | 0.0001 | 0.45 | 0.84 | 0.70 | 0.89 | 0.82 | 0.75 | 0.80 |
| c_Bacilli | 0.0611 | 0.0408 | 0.1678 | 0.0745 | 0.0000 | 2.75 | 0.96 | 0.91 | 0.91 | 0.98 | 0.97 | 0.87 |
| c_Deinococci | 0.0006 | 0.0019 | 0.0017 | 0.0033 | 0.0000 | 2.80 | 0.85 | 0.68 | 0.92 | 0.84 | 0.76 | 0.87 |
| c_TM7-3 | 0.0009 | 0.0027 | 0.0028 | 0.0044 | 0.0000 | 3.02 | 0.85 | 0.68 | 0.91 | 0.85 | 0.75 | 0.87 |
| c_Flavobacteriia | 0.0018 | 0.0066 | 0.0059 | 0.0075 | 0.0000 | 3.28 | 0.85 | 0.68 | 0.93 | 0.86 | 0.75 | 0.87 |
| c_Alphaproteobacteria | 0.0163 | 0.0323 | 0.0632 | 0.0343 | 0.0000 | 3.87 | 0.95 | 0.88 | 0.88 | 0.96 | 0.93 | 0.88 |
| c_Verrucomicrobiae | 0.0052 | 0.0060 | 0.0205 | 0.0140 | 0.0000 | 3.95 | 0.93 | 0.87 | 0.83 | 0.94 | 0.90 | 0.78 |
| c_Fusobacteriia | 0.0009 | 0.0029 | 0.0041 | 0.0055 | 0.0000 | 4.42 | 0.88 | 0.68 | 0.90 | 0.88 | 0.76 | 0.85 |
| c_[Saprospirae] | 0.0002 | 0.0006 | 0.0009 | 0.0027 | 0.0009 | 4.46 | 0.84 | 0.68 | 0.92 | 0.85 | 0.74 | 0.83 |
| c_Sphingobacteriia | 0.0004 | 0.0011 | 0.0019 | 0.0042 | 0.0000 | 4.98 | 0.86 | 0.68 | 0.90 | 0.86 | 0.74 | 0.82 |
| c_Chloroplast | 0.0027 | 0.0049 | 0.0151 | 0.0196 | 0.0000 | 5.54 | 0.91 | 0.79 | 0.87 | 0.91 | 0.81 | 0.80 |
| c_Cytophagia | 0.0002 | 0.0008 | 0.0017 | 0.0050 | 0.0000 | 8.49 | 0.85 | 0.68 | 0.88 | 0.86 | 0.75 | 0.85 |
| c_[Fimbriimonadia] | 0.0001 | 0.0004 | 0.0010 | 0.0027 | 0.0000 | 9.14 | 0.85 | 0.68 | 0.90 | 0.86 | 0.75 | 0.83 |
| c_Thermomicrobia | 0.0000 | 0.0002 | 0.0006 | 0.0018 | 0.0000 | 12.13 | 0.85 | 0.67 | 0.90 | 0.85 | 0.74 | 0.83 |
| c_Solibacteres | 0.0000 | 0.0002 | 0.0010 | 0.0028 | 0.0000 | 31.46 | 0.85 | 0.67 | 0.90 | 0.84 | 0.72 | 0.85 |

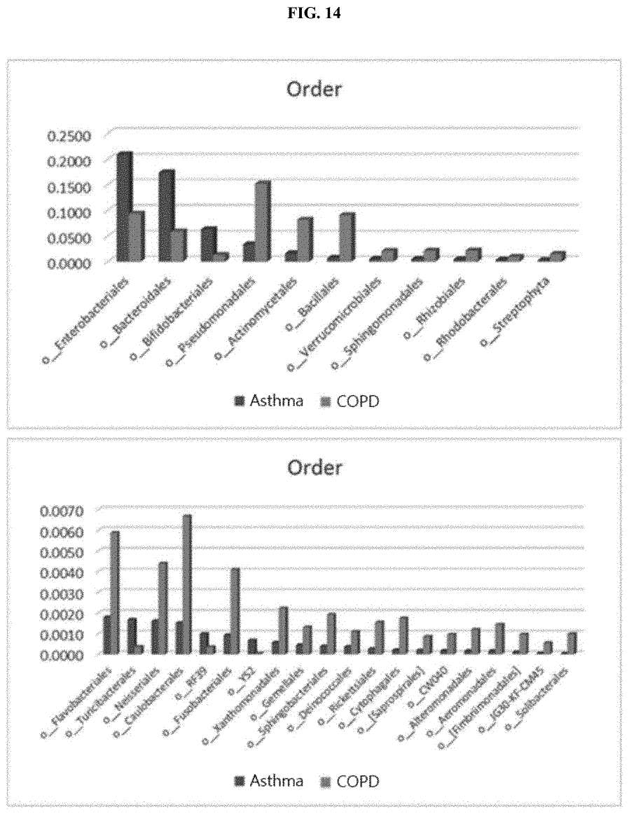

As a result of analyzing bacteria-derived extracellular vesicles in blood at an order level, a diagnostic model developed using, as a biomarker, one or more bacteria from the order YS2, the order Bifidobacteriales, the order Turicibacterales, the order Bacteroidales, the order RF39, the order Enterobacteriales, the order Rhodobacterales, the order Neisseriales, the order Gemellales, the order Deinococcales, the order Flavobacteriales, the order Xanthomonadales, the order Verrucomicrobiales, the order Sphingomonadales, the order Caulobacterales, the order Fusobacteriales, the order Saprospirales, the order Pseudomonadales, the order Sphingobacteriales, the order Rhizobiales, the order Actinomycetales, the order CW040, the order Streptophyta, the order Rickettsiales, the order Alteromonadales, the order Cytophagales, the order Aeromonadales, the order Fimbriimonadales, the order JG30-KF-CM45, the order Bacillales, and the order Solibacterales exhibited significant differential diagnostic performance for asthma and COPD (see Table 14 and FIG. 14).

TABLE 14

| | asthma | | COPD | | t-test | | Training Set | | | Test Set | | |
|---|---|---|---|---|---|---|---|---|---|---|---|---|
| Taxon | Mean | SD | Mean | SD | p-value | Ratio | AUC | sensitivity | specificity | AUC | sensitivity | specificity |
| o_YS2 | 0.0007 | 0.0014 | 0.0000 | 0.0002 | 0.0000 | 0.04 | 0.88 | 0.74 | 0.93 | 0.87 | 0.78 | 0.92 |
| o_Bifidobacteriales | 0.0627 | 0.0335 | 0.0127 | 0.0101 | 0.0000 | 0.20 | 0.97 | 0.89 | 0.95 | 1.00 | 0.99 | 0.93 |
| o_Turicibacterales | 0.0017 | 0.0053 | 0.0004 | 0.0014 | 0.0005 | 0.22 | 0.87 | 0.70 | 0.90 | 0.81 | 0.75 | 0.80 |
| o_Bacteroidales | 0.1736 | 0.0514 | 0.0585 | 0.0261 | 0.0000 | 0.34 | 0.98 | 0.94 | 0.97 | 0.99 | 0.99 | 0.93 |
| o_RF39 | 0.0010 | 0.0016 | 0.0004 | 0.0011 | 0.0000 | 0.36 | 0.85 | 0.72 | 0.88 | 0.82 | 0.75 | 0.77 |
| o_Enterobacteriales | 0.2091 | 0.0878 | 0.0932 | 0.0398 | 0.0000 | 0.45 | 0.94 | 0.83 | 0.93 | 0.97 | 0.94 | 0.85 |
| o_Rhodobacterales | 0.0034 | 0.0237 | 0.0090 | 0.0098 | 0.0014 | 2.66 | 0.84 | 0.66 | 0.90 | 0.84 | 0.74 | 0.85 |
| o_Neisseriales | 0.0016 | 0.0064 | 0.0044 | 0.0052 | 0.0000 | 2.72 | 0.87 | 0.68 | 0.90 | 0.85 | 0.74 | 0.82 |
| o_Gemellales | 0.0004 | 0.0032 | 0.0013 | 0.0028 | 0.0034 | 2.92 | 0.83 | 0.66 | 0.91 | 0.84 | 0.74 | 0.85 |
| o_Deinococcales | 0.0004 | 0.0013 | 0.0011 | 0.0026 | 0.0004 | 2.99 | 0.83 | 0.66 | 0.90 | 0.84 | 0.74 | 0.87 |
| o_Flavobacteriales | 0.0018 | 0.0066 | 0.0059 | 0.0075 | 0.0000 | 3.28 | 0.85 | 0.68 | 0.93 | 0.86 | 0.75 | 0.87 |
| o_Xanthomonadales | 0.0006 | 0.0012 | 0.0022 | 0.0030 | 0.0000 | 3.90 | 0.86 | 0.68 | 0.90 | 0.87 | 0.75 | 0.82 |
| o_Verrucomicrobiales | 0.0052 | 0.0060 | 0.0205 | 0.0140 | 0.0000 | 3.95 | 0.93 | 0.87 | 0.83 | 0.94 | 0.90 | 0.78 |
| o_Sphingomonadales | 0.0049 | 0.0082 | 0.0210 | 0.0208 | 0.0000 | 4.25 | 0.93 | 0.85 | 0.86 | 0.93 | 0.90 | 0.80 |
| o_Caulobacterales | 0.0015 | 0.0030 | 0.0067 | 0.0068 | 0.0000 | 4.41 | 0.91 | 0.80 | 0.88 | 0.91 | 0.79 | 0.80 |
| o_Fusobacteriales | 0.0009 | 0.0029 | 0.0041 | 0.0055 | 0.0000 | 4.42 | 0.88 | 0.68 | 0.90 | 0.88 | 0.76 | 0.85 |
| o_[Saprospirales] | 0.0002 | 0.0006 | 0.0009 | 0.0027 | 0.0009 | 4.46 | 0.84 | 0.68 | 0.92 | 0.85 | 0.74 | 0.83 |
| o_Pseudomonadales | 0.0335 | 0.0496 | 0.1520 | 0.0710 | 0.0000 | 4.53 | 0.97 | 0.93 | 0.94 | 0.98 | 0.97 | 0.90 |
| o_Sphingobacteriales | 0.0004 | 0.0011 | 0.0019 | 0.0042 | 0.0000 | 4.98 | 0.86 | 0.68 | 0.90 | 0.86 | 0.74 | 0.82 |
| o_Rhizobiales | 0.0043 | 0.0057 | 0.0215 | 0.0141 | 0.0000 | 5.02 | 0.95 | 0.90 | 0.88 | 0.96 | 0.93 | 0.82 |
| o_Actinomycetales | 0.0160 | 0.0218 | 0.0812 | 0.0405 | 0.0000 | 5.07 | 0.98 | 0.95 | 0.92 | 0.99 | 0.99 | 0.88 |
| o_CW040 | 0.0002 | 0.0009 | 0.0010 | 0.0025 | 0.0000 | 5.45 | 0.85 | 0.68 | 0.90 | 0.85 | 0.74 | 0.85 |
| o_Streptophyta | 0.0027 | 0.0048 | 0.0149 | 0.0195 | 0.0000 | 5.61 | 0.91 | 0.79 | 0.87 | 0.92 | 0.82 | 0.80 |
| o_Rickettsiales | 0.0003 | 0.0011 | 0.0015 | 0.0027 | 0.0000 | 5.86 | 0.86 | 0.70 | 0.86 | 0.85 | 0.75 | 0.82 |
| o_Alteromonadales | 0.0002 | 0.0007 | 0.0012 | 0.0030 | 0.0000 | 7.38 | 0.86 | 0.68 | 0.90 | 0.86 | 0.74 | 0.85 |
| o_Cytophagales | 0.0002 | 0.0008 | 0.0017 | 0.0050 | 0.0000 | 8.49 | 0.85 | 0.68 | 0.88 | 0.86 | 0.75 | 0.85 |
| o_Aeromonadales | 0.0002 | 0.0007 | 0.0014 | 0.0042 | 0.0000 | 8.93 | 0.86 | 0.70 | 0.90 | 0.85 | 0.74 | 0.75 |
| o_[Fimbriimonadales] | 0.0001 | 0.0004 | 0.0010 | 0.0027 | 0.0000 | 9.14 | 0.85 | 0.68 | 0.90 | 0.86 | 0.75 | 0.83 |
| o_JG30-KF-CM45 | 0.0000 | 0.0002 | 0.0005 | 0.0017 | 0.0001 | 11.57 | 0.85 | 0.67 | 0.90 | 0.85 | 0.74 | 0.83 |
| o_Bacillales | 0.0071 | 0.0085 | 0.0903 | 0.0740 | 0.0000 | 12.76 | 0.99 | 0.99 | 0.98 | 0.99 | 0.97 | 0.97 |
| o_Solibacterales | 0.0000 | 0.0002 | 0.0010 | 0.0028 | 0.0000 | 31.46 | 0.85 | 0.67 | 0.90 | 0.84 | 0.72 | 0.85 |

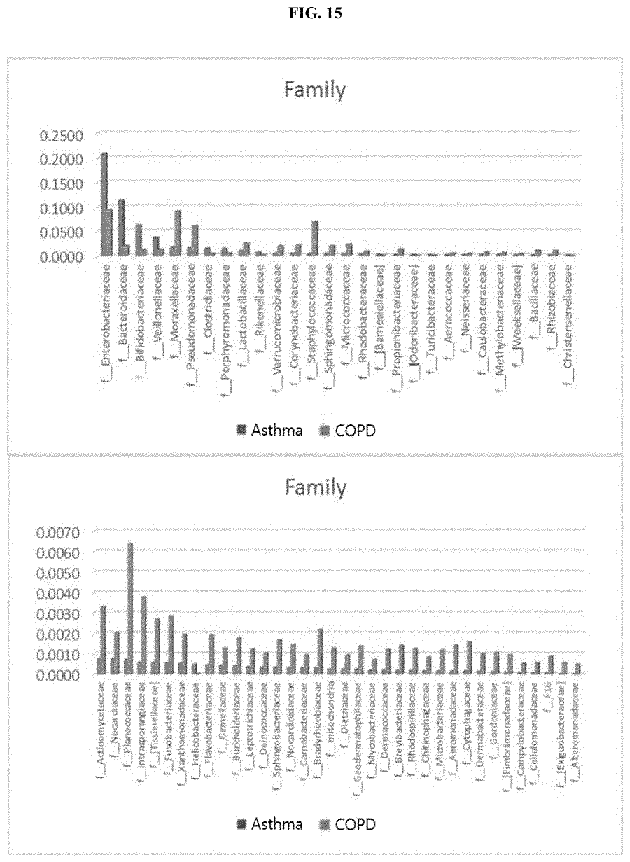

As a result of analyzing bacteria-derived extracellular vesicles in blood at a family level, a diagnostic model developed using, as a biomarker, one or more bacteria from the family Helicobacteraceae, the family Bacteroidaceae, the family Bifidobacteriaceae, the family Turicibacteraceae, the family Rikenellaceae, the family Odoribacteraceae, the family Clostridiaceae, the family Barnesiellaceae, the family Veillonellaceae, the family Porphyromonadaceae, the family Enterobacteriaceae, the family Christensenellaceae, the family Lactobacillaceae, the family Rhodobacteraceae, the family Nocardiaceae, the family Neisseriaceae, the family Gemellaceae, the family Carnobacteriaceae, the family Aerococcaceae, the family Weeksellaceae, the family Deinococcaceae, the family Leptotrichiaceae, the family Mycobacteriaceae, the family Dietziaceae, the family Xanthomonadaceae, the family Pseudomonadaceae, the family Verrucomicrobiaceae, the family Methylobacteriaceae, the family Flavobacteriaceae, the family Actinomycetaceae, the family Burkholderiaceae, the family Nocardioidaceae, the family Caulobacteraceae, the family Sphingomonadaceae, the family Corynebacteriaceae, the family Tissierellaceae, the family Chitinophagaceae, the family mitochondria, the family Sphingobacteriaceae, the family Fusobacteriaceae, the family Moraxellaceae, the family Micrococcaceae, the family Geodermatophilaceae, the family Dermacoccaceae, the family Intrasporangiaceae, the family Dermabacteraceae, the family Propionibacteriaceae, the family Rhodospirillaceae, the family Bradyrhizobiaceae, the family Campylobacteraceae, the family Brevibacteriaceae, the family Microbacteriaceae, the family Cellulomonadaceae, the family Gordoniaceae, the family Bacillaceae, the family Planococcaceae, the family Rhizobiaceae, the family Aeromonadaceae, the family Fimbriimonadaceae, the family Cytophagaceae, the family F16, the family Staphylococcaceae, the family Exiguobacteraceae, and the family Alteromonadaceae exhibited significant differential diagnostic performance for asthma and COPD (see Table 15 and FIG. 15).

TABLE 15

| | asthma | | COPD | | t-test | | Training Set | | | Test Set | | |
|---|---|---|---|---|---|---|---|---|---|---|---|---|
| Taxon | Mean | SD | Mean | SD | p-value | Ratio | AUC | sensitivity | specificity | AUC | sensitivity | specificity |
| f_Helicobacteraceae | 0.0005 | 0.0022 | 0.0000 | 0.0004 | 0.0040 | 0.09 | 0.84 | 0.67 | 0.90 | 0.82 | 0.74 | 0.85 |
| f_Bacteroidaceae | 0.1140 | 0.0469 | 0.0209 | 0.0143 | 0.0000 | 0.18 | 0.98 | 0.93 | 0.96 | 0.99 | 0.97 | 0.95 |
| f_Bifidobacteriaceae | 0.0627 | 0.0335 | 0.0127 | 0.0101 | 0.0000 | 0.20 | 0.97 | 0.89 | 0.95 | 1.00 | 0.99 | 0.93 |
| f_Turicibacteraceae | 0.0017 | 0.0053 | 0.0004 | 0.0014 | 0.0005 | 0.22 | 0.87 | 0.70 | 0.90 | 0.81 | 0.75 | 0.80 |
| f_Rikenellaceae | 0.0070 | 0.0087 | 0.0017 | 0.0029 | 0.0000 | 0.24 | 0.90 | 0.75 | 0.89 | 0.88 | 0.78 | 0.90 |
| f_[Odoribacteraceae] | 0.0021 | 0.0029 | 0.0007 | 0.0019 | 0.0000 | 0.31 | 0.86 | 0.70 | 0.92 | 0.86 | 0.75 | 0.92 |
| f_Clostridiaceae | 0.0151 | 0.0143 | 0.0048 | 0.0062 | 0.0000 | 0.32 | 0.90 | 0.75 | 0.90 | 0.83 | 0.75 | 0.85 |
| f_[Barnesiellaceae] | 0.0024 | 0.0050 | 0.0008 | 0.0022 | 0.0000 | 0.34 | 0.85 | 0.66 | 0.89 | 0.84 | 0.74 | 0.85 |
| f_Veillonellaceae | 0.0375 | 0.0288 | 0.0128 | 0.0154 | 0.0000 | 0.34 | 0.92 | 0.79 | 0.88 | 0.94 | 0.87 | 0.92 |
| f_Porphyromonadaceae | 0.0145 | 0.0090 | 0.0052 | 0.0048 | 0.0000 | 0.36 | 0.93 | 0.82 | 0.89 | 0.92 | 0.90 | 0.83 |
| f_Enterobacteriaceae | 0.2091 | 0.0878 | 0.0932 | 0.0398 | 0.0000 | 0.45 | 0.94 | 0.83 | 0.93 | 0.97 | 0.94 | 0.85 |
| f_Christensenellaceae | 0.0010 | 0.0018 | 0.0005 | 0.0019 | 0.0038 | 0.50 | 0.83 | 0.65 | 0.87 | 0.83 | 0.72 | 0.88 |
| f_Lactobacillaceae | 0.0108 | 0.0141 | 0.0256 | 0.0264 | 0.0000 | 2.38 | 0.89 | 0.75 | 0.90 | 0.92 | 0.79 | 0.87 |
| f_Rhodobacteraceae | 0.0034 | 0.0237 | 0.0089 | 0.0098 | 0.0016 | 2.64 | 0.84 | 0.66 | 0.90 | 0.84 | 0.74 | 0.85 |
| f_Nocardiaceae | 0.0008 | 0.0019 | 0.0020 | 0.0028 | 0.0000 | 2.69 | 0.87 | 0.70 | 0.90 | 0.86 | 0.75 | 0.78 |
| f_Neisseriaceae | 0.0016 | 0.0064 | 0.0044 | 0.0052 | 0.0000 | 2.72 | 0.87 | 0.68 | 0.90 | 0.85 | 0.74 | 0.82 |
| f_Gemellaceae | 0.0004 | 0.0032 | 0.0013 | 0.0028 | 0.0041 | 2.90 | 0.83 | 0.66 | 0.91 | 0.83 | 0.72 | 0.85 |
| f_Carnobacteriaceae | 0.0003 | 0.0012 | 0.0009 | 0.0019 | 0.0001 | 2.90 | 0.84 | 0.66 | 0.91 | 0.83 | 0.74 | 0.83 |
| f_Aerococcaceae | 0.0017 | 0.0042 | 0.0049 | 0.0057 | 0.0000 | 2.95 | 0.88 | 0.73 | 0.87 | 0.88 | 0.76 | 0.80 |
| f_[Weeksellaceae] | 0.0013 | 0.0061 | 0.0040 | 0.0069 | 0.0000 | 2.98 | 0.83 | 0.66 | 0.92 | 0.84 | 0.74 | 0.87 |
| f_Deinococcaceae | 0.0003 | 0.0013 | 0.0010 | 0.0026 | 0.0006 | 3.04 | 0.83 | 0.66 | 0.90 | 0.84 | 0.74 | 0.87 |
| f_Leptotrichiaceae | 0.0004 | 0.0011 | 0.0012 | 0.0029 | 0.0001 | 3.41 | 0.85 | 0.67 | 0.90 | 0.84 | 0.75 | 0.83 |
| f_Mycobacteriaceae | 0.0002 | 0.0009 | 0.0007 | 0.0018 | 0.0003 | 3.45 | 0.83 | 0.66 | 0.90 | 0.84 | 0.74 | 0.87 |
| f_Dietziaceae | 0.0003 | 0.0008 | 0.0009 | 0.0021 | 0.0000 | 3.62 | 0.84 | 0.66 | 0.92 | 0.84 | 0.74 | 0.82 |
| f_Xanthomonadaceae | 0.0005 | 0.0012 | 0.0019 | 0.0028 | 0.0000 | 3.63 | 0.85 | 0.67 | 0.91 | 0.87 | 0.75 | 0.85 |
| f_Pseudomonadaceae | 0.0160 | 0.0154 | 0.0612 | 0.0353 | 0.0000 | 3.82 | 0.97 | 0.93 | 0.90 | 0.97 | 0.91 | 0.88 |
| f_Verrucomicrobiaceae | 0.0052 | 0.0060 | 0.0205 | 0.0140 | 0.0000 | 3.95 | 0.93 | 0.87 | 0.83 | 0.94 | 0.90 | 0.78 |
| f_Methylobacteriaceae | 0.0015 | 0.0034 | 0.0060 | 0.0067 | 0.0000 | 4.06 | 0.90 | 0.74 | 0.88 | 0.88 | 0.78 | 0.78 |
| f_Flavobacteriaceae | 0.0005 | 0.0022 | 0.0019 | 0.0033 | 0.0000 | 4.10 | 0.84 | 0.66 | 0.92 | 0.85 | 0.74 | 0.90 |
| f_Actinomycetaceae | 0.0008 | 0.0038 | 0.0033 | 0.0044 | 0.0000 | 4.18 | 0.87 | 0.68 | 0.92 | 0.88 | 0.76 | 0.85 |
| f_Burkholderiaceae | 0.0004 | 0.0010 | 0.0018 | 0.0035 | 0.0000 | 4.35 | 0.85 | 0.67 | 0.90 | 0.84 | 0.74 | 0.85 |
| f_Nocardioidaceae | 0.0003 | 0.0008 | 0.0015 | 0.0030 | 0.0000 | 4.36 | 0.86 | 0.67 | 0.87 | 0.84 | 0.75 | 0.77 |
| f_Caulobacteraceae | 0.0015 | 0.0030 | 0.0067 | 0.0068 | 0.0000 | 4.41 | 0.91 | 0.80 | 0.88 | 0.91 | 0.79 | 0.80 |
| f_Sphingomonadaceae | 0.0046 | 0.0070 | 0.0204 | 0.0206 | 0.0000 | 4.41 | 0.94 | 0.87 | 0.83 | 0.94 | 0.90 | 0.82 |
| f_Corynebacteriaceae | 0.0048 | 0.0055 | 0.0216 | 0.0234 | 0.0000 | 4.50 | 0.92 | 0.84 | 0.86 | 0.94 | 0.91 | 0.80 |
| f_[Tissierellaceae] | 0.0006 | 0.0016 | 0.0027 | 0.0043 | 0.0000 | 4.62 | 0.88 | 0.72 | 0.85 | 0.87 | 0.78 | 0.82 |
| f_Chitinophagaceae | 0.0002 | 0.0006 | 0.0009 | 0.0027 | 0.0007 | 4.87 | 0.84 | 0.68 | 0.92 | 0.85 | 0.76 | 0.83 |
| f_mitochondria | 0.0003 | 0.0011 | 0.0013 | 0.0025 | 0.0000 | 4.91 | 0.85 | 0.68 | 0.89 | 0.84 | 0.75 | 0.83 |
| f_Sphingobacteriaceae | 0.0003 | 0.0010 | 0.0017 | 0.0041 | 0.0000 | 4.94 | 0.85 | 0.68 | 0.91 | 0.86 | 0.74 | 0.85 |
| f_Fusobacteriaceae | 0.0006 | 0.0026 | 0.0029 | 0.0042 | 0.0000 | 5.08 | 0.87 | 0.68 | 0.90 | 0.87 | 0.76 | 0.87 |
| f_Moraxellaceae | 0.0175 | 0.0451 | 0.0908 | 0.0628 | 0.0000 | 5.20 | 0.96 | 0.91 | 0.92 | 0.97 | 0.96 | 0.88 |
| f_Micrococcaceae | 0.0045 | 0.0143 | 0.0236 | 0.0165 | 0.0000 | 5.22 | 0.96 | 0.91 | 0.88 | 0.97 | 0.94 | 0.87 |
| f_Geodermatophilaceae | 0.0003 | 0.0012 | 0.0014 | 0.0028 | 0.0000 | 5.32 | 0.86 | 0.68 | 0.89 | 0.86 | 0.75 | 0.80 |
| f_Dermacoccaceae | 0.0002 | 0.0005 | 0.0012 | 0.0025 | 0.0000 | 5.92 | 0.85 | 0.68 | 0.91 | 0.86 | 0.76 | 0.87 |
| f_Intrasporangiaceae | 0.0006 | 0.0013 | 0.0038 | 0.0042 | 0.0000 | 6.29 | 0.91 | 0.79 | 0.83 | 0.90 | 0.82 | 0.73 |
| f_Dermabacteraceae | 0.0002 | 0.0005 | 0.0010 | 0.0019 | 0.0000 | 6.51 | 0.87 | 0.68 | 0.90 | 0.86 | 0.76 | 0.83 |
| f_Propionibacteriaceae | 0.0021 | 0.0031 | 0.0140 | 0.0109 | 0.0000 | 6.52 | 0.97 | 0.95 | 0.90 | 0.94 | 0.87 | 0.83 |
| f_Rhodospirillaceae | 0.0002 | 0.0011 | 0.0013 | 0.0058 | 0.0097 | 6.56 | 0.83 | 0.66 | 0.91 | 0.84 | 0.74 | 0.85 |
| f_Bradyrhizobiaceae | 0.0003 | 0.0008 | 0.0022 | 0.0039 | 0.0000 | 6.80 | 0.87 | 0.70 | 0.90 | 0.86 | 0.76 | 0.77 |
| f_Campylobacteraceae | 0.0001 | 0.0004 | 0.0006 | 0.0024 | 0.0055 | 6.84 | 0.84 | 0.66 | 0.90 | 0.83 | 0.74 | 0.83 |
| f_Brevibacteriaceae | 0.0002 | 0.0006 | 0.0014 | 0.0032 | 0.0000 | 7.13 | 0.86 | 0.69 | 0.88 | 0.85 | 0.76 | 0.80 |
| f_Microbacteriaceae | 0.0002 | 0.0008 | 0.0012 | 0.0032 | 0.0000 | 7.18 | 0.89 | 0.75 | 0.86 | 0.86 | 0.75 | 0.83 |
| f_Cellulomonadaceae | 0.0001 | 0.0004 | 0.0006 | 0.0018 | 0.0002 | 7.19 | 0.84 | 0.67 | 0.91 | 0.83 | 0.74 | 0.83 |
| f_Gordoniaceae | 0.0001 | 0.0005 | 0.0011 | 0.0026 | 0.0000 | 8.05 | 0.84 | 0.66 | 0.89 | 0.85 | 0.74 | 0.83 |
| f_Bacillaceae | 0.0013 | 0.0020 | 0.0112 | 0.0146 | 0.0000 | 8.42 | 0.95 | 0.89 | 0.83 | 0.94 | 0.91 | 0.77 |
| f_Planococcaceae | 0.0007 | 0.0013 | 0.0064 | 0.0098 | 0.0000 | 8.73 | 0.95 | 0.91 | 0.87 | 0.94 | 0.94 | 0.78 |
| f_Rhizobiaceae | 0.0012 | 0.0025 | 0.0105 | 0.0095 | 0.0000 | 8.90 | 0.95 | 0.89 | 0.83 | 0.96 | 0.96 | 0.85 |
| f_Aeromonadaceae | 0.0002 | 0.0007 | 0.0014 | 0.0042 | 0.0000 | 8.92 | 0.86 | 0.70 | 0.90 | 0.84 | 0.74 | 0.75 |
| f_[Fimbriimonadaceae] | 0.0001 | 0.0004 | 0.0010 | 0.0027 | 0.0000 | 9.14 | 0.85 | 0.68 | 0.90 | 0.86 | 0.75 | 0.83 |
| f_Cytophagaceae | 0.0002 | 0.0005 | 0.0016 | 0.0043 | 0.0000 | 10.10 | 0.85 | 0.68 | 0.88 | 0.87 | 0.76 | 0.85 |
| f_F16 | 0.0001 | 0.0005 | 0.0009 | 0.0023 | 0.0000 | 13.46 | 0.85 | 0.68 | 0.90 | 0.86 | 0.76 | 0.85 |
| f_Staphylococcaceae | 0.0047 | 0.0068 | 0.0700 | 0.0711 | 0.0000 | 14.90 | 0.99 | 0.97 | 0.96 | 0.99 | 0.97 | 0.95 |
| f_[Exiguobacteraceae] | 0.0000 | 0.0002 | 0.0006 | 0.0030 | 0.0085 | 24.47 | 0.85 | 0.67 | 0.91 | 0.86 | 0.76 | 0.87 |
| f_Alteromonadaceae | 0.0000 | 0.0001 | 0.0005 | 0.0018 | 0.0001 | 50.69 | 0.85 | 0.67 | 0.92 | 0.86 | 0.74 | 0.88 |

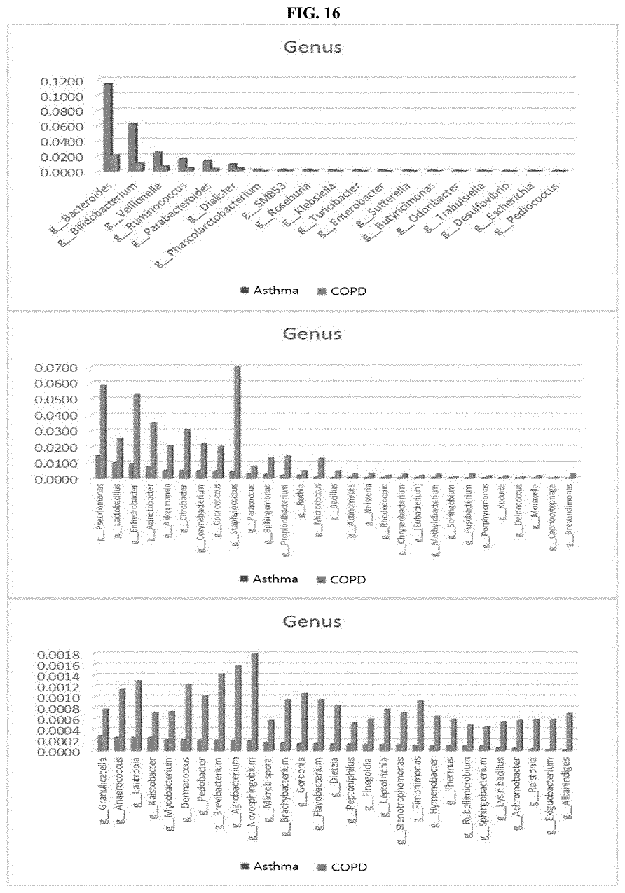

As a result of analyzing bacteria-derived extracellular vesicles in blood at a genus level, a diagnostic model developed using, as a biomarker, one or more bacteria from the genus Enterobacter, the genus Trabulsiella, the genus Phascolarctobacterium, the genus Klebsiella, the genus Bifidobacterium, the genus Bacteroides, the genus Turicibacter, the genus Sutterella, the genus Butyricimonas, the genus Parabacteroides, the genus Ruminococcus, the genus Veillonella, the genus Pediococcus, the genus Desulfovibrio, the genus SMB53, the genus Roseburia, the genus Odoribacter, the genus Dialister, the genus Escherichia, the genus Sphingobium, the genus Rothia, the genus Paracoccus, the genus Lactobacillus, the genus Rhodococcus, the genus Eubacterium, the genus Granulicatella, the genus Kaistobacter, the genus Capnocytophaga, the genus Deinococcus, the genus Mycobacterium, the genus Microbispora, the genus Methylobacterium, the genus Chryseobacterium, the genus Actinomyces, the genus Porphyromonas, the genus Kocuria, the genus Akkermansia, the genus Pseudomonas, the genus Coprococcus, the genus Peptoniphilus, the genus Neisseria, the genus Corynebacterium, the genus Anaerococcus, the genus Acinetobacter, the genus Rubellimicrobium, the genus Sphingobacterium, the genus Sphingomonas, the genus Pedobacter, the genus Finegoldia, the genus Fusobacterium, the genus Lautropia, the genus Moraxella, the genus Enhydrobacter, the genus Dermacoccus, the genus Thermus, the genus Citrobacter, the genus Bacillus, the genus Stenotrophomonas, the genus Hymenobacter, the genus Brachybacterium, the genus Propionibacterium, the genus Leptotrichia, the genus Dietzia, the genus Brevibacterium, the genus Flavobacterium, the genus Gordonia, the genus Agrobacterium, the genus Fimbriimonas, the genus Novosphingobium, the genus Lysinibacillus, the genus Brevundimonas, the genus Achromobacter, the genus Micrococcus, the genus Staphylococcus, the genus Ralstonia, the genus Exiguobacterium, and the genus Alkanindiges exhibited significant differential diagnostic performance for asthma and COPD (see Table 16 and FIG. 16).

TABLE 16

| | asthma | | COPD | | t-test | | Training Set | | | Test Set | | |
|---|---|---|---|---|---|---|---|---|---|---|---|---|
| Taxon | Mean | SD | Mean | SD | p-value | Ratio | AUC | sensitivity | specificity | AUC | sensitivity | specificity |
| g_Enterobacter | 0.0016 | 0.0013 | 0.0001 | 0.0005 | 0.0000 | 0.08 | 0.96 | 0.91 | 0.94 | 0.95 | 0.94 | 0.85 |
| g_Trabulsiella | 0.0007 | 0.0012 | 0.0001 | 0.0004 | 0.0000 | 0.08 | 0.93 | 0.82 | 0.93 | 0.95 | 0.96 | 0.87 |
| g_Phascolarctobacterium | 0.0024 | 0.0101 | 0.0003 | 0.0010 | 0.0026 | 0.12 | 0.88 | 0.72 | 0.90 | 0.89 | 0.78 | 0.90 |
| g_Klebsiella | 0.0018 | 0.0012 | 0.0003 | 0.0006 | 0.0000 | 0.16 | 0.96 | 0.87 | 0.92 | 0.97 | 0.94 | 0.90 |
| g_Bifidobacterium | 0.0623 | 0.0336 | 0.0105 | 0.0083 | 0.0000 | 0.17 | 0.97 | 0.89 | 0.96 | 1.00 | 0.99 | 0.95 |
| g_Bacteroides | 0.1140 | 0.0469 | 0.0209 | 0.0143 | 0.0000 | 0.18 | 0.98 | 0.93 | 0.96 | 0.99 | 0.97 | 0.95 |
| g_Turicibacter | 0.0017 | 0.0053 | 0.0004 | 0.0014 | 0.0005 | 0.22 | 0.87 | 0.70 | 0.90 | 0.81 | 0.75 | 0.80 |
| g_Sutterella | 0.0014 | 0.0029 | 0.0003 | 0.0018 | 0.0000 | 0.23 | 0.85 | 0.70 | 0.92 | 0.83 | 0.75 | 0.87 |
| g_Butyricimonas | 0.0012 | 0.0019 | 0.0003 | 0.0009 | 0.0000 | 0.23 | 0.88 | 0.73 | 0.90 | 0.86 | 0.75 | 0.87 |
| g_Parabacteroides | 0.0138 | 0.0086 | 0.0032 | 0.0038 | 0.0000 | 0.23 | 0.95 | 0.85 | 0.88 | 0.96 | 0.90 | 0.87 |
| g_Ruminococcus | 0.0163 | 0.0112 | 0.0042 | 0.0058 | 0.0000 | 0.26 | 0.94 | 0.84 | 0.88 | 0.94 | 0.87 | 0.82 |
| g_Veillonella | 0.0243 | 0.0248 | 0.0064 | 0.0127 | 0.0000 | 0.26 | 0.91 | 0.77 | 0.90 | 0.94 | 0.84 | 0.93 |
| g_Pediococcus | 0.0004 | 0.0010 | 0.0001 | 0.0008 | 0.0006 | 0.28 | 0.83 | 0.64 | 0.90 | 0.83 | 0.72 | 0.88 |
| g_Desulfovibrio | 0.0005 | 0.0016 | 0.0001 | 0.0006 | 0.0016 | 0.28 | 0.82 | 0.65 | 0.90 | 0.82 | 0.74 | 0.88 |
| g_SMB53 | 0.0023 | 0.0033 | 0.0007 | 0.0016 | 0.0000 | 0.32 | 0.86 | 0.72 | 0.90 | 0.81 | 0.75 | 0.85 |
| g_Roseburia | 0.0021 | 0.0029 | 0.0007 | 0.0014 | 0.0000 | 0.33 | 0.86 | 0.69 | 0.88 | 0.86 | 0.76 | 0.83 |
| g_Odoribacter | 0.0009 | 0.0017 | 0.0004 | 0.0017 | 0.0028 | 0.44 | 0.83 | 0.68 | 0.90 | 0.84 | 0.74 | 0.87 |
| g_Dialister | 0.0090 | 0.0077 | 0.0041 | 0.0066 | 0.0000 | 0.46 | 0.86 | 0.69 | 0.89 | 0.87 | 0.76 | 0.85 |
| g_Escherichia | 0.0005 | 0.0007 | 0.0003 | 0.0003 | 0.0000 | 0.50 | 0.84 | 0.66 | 0.91 | 0.86 | 0.76 | 0.85 |
| g_Sphingobium | 0.0006 | 0.0022 | 0.0012 | 0.0024 | 0.0037 | 2.15 | 0.83 | 0.66 | 0.90 | 0.84 | 0.74 | 0.87 |
| g_Rothia | 0.0021 | 0.0139 | 0.0048 | 0.0058 | 0.0098 | 2.26 | 0.83 | 0.65 | 0.90 | 0.83 | 0.72 | 0.87 |
| g_Paracoccus | 0.0031 | 0.0235 | 0.0078 | 0.0095 | 0.0076 | 2.47 | 0.84 | 0.66 | 0.90 | 0.83 | 0.74 | 0.85 |
| g_Lactobacillus | 0.0101 | 0.0140 | 0.0252 | 0.0264 | 0.0000 | 2.49 | 0.89 | 0.75 | 0.90 | 0.92 | 0.79 | 0.87 |
| g_Rhodococcus | 0.0008 | 0.0019 | 0.0020 | 0.0028 | 0.0000 | 2.71 | 0.87 | 0.70 | 0.90 | 0.86 | 0.75 | 0.78 |
| g_[Eubacterium] | 0.0007 | 0.0013 | 0.0020 | 0.0034 | 0.0000 | 2.75 | 0.86 | 0.70 | 0.89 | 0.85 | 0.78 | 0.82 |
| g_Granulicatella | 0.0003 | 0.0011 | 0.0008 | 0.0019 | 0.0010 | 2.80 | 0.84 | 0.66 | 0.90 | 0.83 | 0.74 | 0.83 |
| g_Kaistobacter | 0.0002 | 0.0010 | 0.0007 | 0.0018 | 0.0017 | 2.86 | 0.84 | 0.66 | 0.90 | 0.84 | 0.74 | 0.87 |
| g_Capnocytophaga | 0.0003 | 0.0021 | 0.0009 | 0.0021 | 0.0060 | 2.91 | 0.82 | 0.65 | 0.90 | 0.82 | 0.72 | 0.87 |
| g_Deinococcus | 0.0003 | 0.0013 | 0.0010 | 0.0026 | 0.0007 | 3.01 | 0.83 | 0.66 | 0.90 | 0.84 | 0.74 | 0.87 |
| g_Mycobacterium | 0.0002 | 0.0009 | 0.0007 | 0.0018 | 0.0003 | 3.45 | 0.83 | 0.66 | 0.90 | 0.84 | 0.74 | 0.87 |
| g_Microbispora | 0.0002 | 0.0006 | 0.0006 | 0.0018 | 0.0026 | 3.58 | 0.83 | 0.65 | 0.91 | 0.83 | 0.72 | 0.88 |
| g_Methylobacterium | 0.0007 | 0.0020 | 0.0026 | 0.0044 | 0.0000 | 3.71 | 0.86 | 0.67 | 0.88 | 0.85 | 0.74 | 0.80 |
| g_Chryseobacterium | 0.0007 | 0.0025 | 0.0027 | 0.0063 | 0.0000 | 3.81 | 0.85 | 0.67 | 0.91 | 0.85 | 0.74 | 0.82 |
| g_Actinomyces | 0.0008 | 0.0038 | 0.0030 | 0.0041 | 0.0000 | 3.85 | 0.87 | 0.68 | 0.91 | 0.87 | 0.76 | 0.85 |
| g_Porphyromonas | 0.0005 | 0.0036 | 0.0018 | 0.0032 | 0.0001 | 3.86 | 0.84 | 0.65 | 0.90 | 0.84 | 0.74 | 0.88 |
| g_Kocuria | 0.0004 | 0.0014 | 0.0017 | 0.0032 | 0.0000 | 3.88 | 0.85 | 0.67 | 0.89 | 0.87 | 0.75 | 0.83 |
| g_Akkermansia | 0.0052 | 0.0060 | 0.0205 | 0.0140 | 0.0000 | 3.95 | 0.93 | 0.87 | 0.83 | 0.94 | 0.90 | 0.78 |
| g_Pseudomonas | 0.0144 | 0.0145 | 0.0584 | 0.0352 | 0.0000 | 4.06 | 0.97 | 0.93 | 0.90 | 0.97 | 0.91 | 0.87 |
| g_Coprococcus | 0.0047 | 0.0050 | 0.0199 | 0.0159 | 0.0000 | 4.19 | 0.91 | 0.80 | 0.81 | 0.95 | 0.84 | 0.87 |
| g_Peptoniphilus | 0.0001 | 0.0005 | 0.0005 | 0.0015 | 0.0003 | 4.20 | 0.84 | 0.67 | 0.90 | 0.84 | 0.74 | 0.83 |
| g_Neisseria | 0.0008 | 0.0026 | 0.0032 | 0.0048 | 0.0000 | 4.27 | 0.87 | 0.70 | 0.90 | 0.86 | 0.76 | 0.80 |
| g_Corynebacterium | 0.0048 | 0.0055 | 0.0216 | 0.0234 | 0.0000 | 4.50 | 0.92 | 0.84 | 0.86 | 0.94 | 0.91 | 0.80 |
| g_Anaerococcus | 0.0003 | 0.0013 | 0.0011 | 0.0028 | 0.0000 | 4.50 | 0.85 | 0.67 | 0.90 | 0.84 | 0.75 | 0.87 |
| g_Acinetobacter | 0.0076 | 0.0128 | 0.0347 | 0.0292 | 0.0000 | 4.58 | 0.95 | 0.91 | 0.92 | 0.96 | 0.91 | 0.88 |
| g_Rubellimicrobium | 0.0001 | 0.0007 | 0.0005 | 0.0016 | 0.0021 | 4.81 | 0.84 | 0.68 | 0.90 | 0.83 | 0.72 | 0.85 |
| g_Sphingobacterium | 0.0001 | 0.0006 | 0.0004 | 0.0016 | 0.0032 | 5.00 | 0.84 | 0.67 | 0.90 | 0.82 | 0.71 | 0.85 |
| g_Sphingomonas | 0.0025 | 0.0040 | 0.0127 | 0.0176 | 0.0000 | 5.01 | 0.93 | 0.84 | 0.82 | 0.93 | 0.90 | 0.83 |
| g_Pedobacter | 0.0002 | 0.0005 | 0.0010 | 0.0035 | 0.0015 | 5.03 | 0.84 | 0.67 | 0.90 | 0.84 | 0.74 | 0.85 |
| g_Finegoldia | 0.0001 | 0.0006 | 0.0006 | 0.0018 | 0.0004 | 5.06 | 0.84 | 0.67 | 0.90 | 0.83 | 0.74 | 0.82 |
| g_Fusobacterium | 0.0006 | 0.0026 | 0.0029 | 0.0042 | 0.0000 | 5.08 | 0.87 | 0.68 | 0.90 | 0.87 | 0.76 | 0.87 |
| g_Lautropia | 0.0002 | 0.0005 | 0.0013 | 0.0030 | 0.0000 | 5.21 | 0.84 | 0.66 | 0.90 | 0.83 | 0.75 | 0.88 |
| g_Moraxella | 0.0003 | 0.0008 | 0.0019 | 0.0054 | 0.0001 | 5.50 | 0.83 | 0.66 | 0.90 | 0.84 | 0.74 | 0.87 |
| g_Enhydrobacter | 0.0093 | 0.0425 | 0.0525 | 0.0478 | 0.0000 | 5.66 | 0.94 | 0.81 | 0.92 | 0.95 | 0.87 | 0.92 |
| g_Dermacoccus | 0.0002 | 0.0005 | 0.0012 | 0.0025 | 0.0000 | 5.92 | 0.85 | 0.68 | 0.91 | 0.86 | 0.76 | 0.87 |
| g_Thermus | 0.0001 | 0.0007 | 0.0006 | 0.0020 | 0.0010 | 5.94 | 0.85 | 0.66 | 0.90 | 0.81 | 0.71 | 0.83 |
| g_Citrobacter | 0.0050 | 0.0041 | 0.0305 | 0.0214 | 0.0000 | 6.09 | 0.95 | 0.91 | 0.85 | 0.97 | 0.93 | 0.88 |
| g_Bacillus | 0.0008 | 0.0016 | 0.0048 | 0.0070 | 0.0000 | 6.15 | 0.90 | 0.78 | 0.83 | 0.88 | 0.79 | 0.73 |
| g_Stenotrophomonas | 0.0001 | 0.0005 | 0.0007 | 0.0016 | 0.0000 | 6.26 | 0.85 | 0.68 | 0.91 | 0.85 | 0.76 | 0.88 |
| g_Hymenobacter | 0.0001 | 0.0004 | 0.0006 | 0.0019 | 0.0002 | 6.35 | 0.84 | 0.66 | 0.90 | 0.83 | 0.74 | 0.85 |
| g_Brachybacterium | 0.0001 | 0.0005 | 0.0009 | 0.0018 | 0.0000 | 6.46 | 0.86 | 0.68 | 0.90 | 0.86 | 0.76 | 0.83 |
| g_Propionibacterium | 0.0021 | 0.0031 | 0.0140 | 0.0109 | 0.0000 | 6.51 | 0.97 | 0.95 | 0.90 | 0.94 | 0.87 | 0.83 |
| g_Leptotrichia | 0.0001 | 0.0005 | 0.0008 | 0.0022 | 0.0001 | 6.65 | 0.85 | 0.67 | 0.90 | 0.84 | 0.75 | 0.83 |
| g_Dietzia | 0.0001 | 0.0006 | 0.0008 | 0.0020 | 0.0000 | 6.84 | 0.84 | 0.66 | 0.92 | 0.84 | 0.76 | 0.82 |
| g_Brevibacterium | 0.0002 | 0.0006 | 0.0014 | 0.0032 | 0.0000 | 7.13 | 0.86 | 0.69 | 0.88 | 0.85 | 0.76 | 0.80 |
| g_Flavobacterium | 0.0001 | 0.0005 | 0.0009 | 0.0021 | 0.0000 | 7.17 | 0.86 | 0.68 | 0.90 | 0.85 | 0.75 | 0.83 |
| g_Gordonia | 0.0001 | 0.0005 | 0.0011 | 0.0026 | 0.0000 | 8.05 | 0.84 | 0.66 | 0.89 | 0.85 | 0.74 | 0.83 |
| g_Agrobacterium | 0.0002 | 0.0007 | 0.0016 | 0.0039 | 0.0000 | 8.07 | 0.86 | 0.67 | 0.90 | 0.85 | 0.75 | 0.83 |
| g_Fimbriimonas | 0.0001 | 0.0004 | 0.0009 | 0.0025 | 0.0000 | 9.15 | 0.85 | 0.68 | 0.90 | 0.86 | 0.75 | 0.83 |
| g_Novosphingobium | 0.0002 | 0.0009 | 0.0018 | 0.0044 | 0.0000 | 9.30 | 0.86 | 0.68 | 0.88 | 0.86 | 0.76 | 0.83 |
| g_Lysinibacillus | 0.0001 | 0.0003 | 0.0005 | 0.0017 | 0.0001 | 9.37 | 0.84 | 0.65 | 0.90 | 0.84 | 0.74 | 0.85 |
| g_Brevundimonas | 0.0003 | 0.0008 | 0.0031 | 0.0050 | 0.0000 | 10.67 | 0.89 | 0.75 | 0.88 | 0.88 | 0.76 | 0.80 |
| g_Achromobacter | 0.0001 | 0.0004 | 0.0006 | 0.0018 | 0.0001 | 10.92 | 0.85 | 0.64 | 0.90 | 0.84 | 0.74 | 0.85 |
| g_Micrococcus | 0.0010 | 0.0017 | 0.0125 | 0.0121 | 0.0000 | 12.87 | 0.96 | 0.95 | 0.86 | 0.96 | 0.94 | 0.87 |
| g_Staphylococcus | 0.0044 | 0.0062 | 0.0694 | 0.0711 | 0.0000 | 15.73 | 0.99 | 0.97 | 0.96 | 0.99 | 0.99 | 0.95 |
| g_Ralstonia | 0.0000 | 0.0001 | 0.0006 | 0.0014 | 0.0000 | 16.54 | 0.86 | 0.68 | 0.91 | 0.85 | 0.76 | 0.85 |
| g_Exiguobacterium | 0.0000 | 0.0002 | 0.0006 | 0.0030 | 0.0083 | 24.69 | 0.85 | 0.67 | 0.91 | 0.86 | 0.76 | 0.87 |
| g_Alkanindiges | 0.0000 | 0.0001 | 0.0007 | 0.0032 | 0.0027 | 55.07 | 0.85 | 0.68 | 0.91 | 0.85 | 0.76 | 0.85 |

The above description of the present invention is provided only for illustrative purposes, and it will be understood by one of ordinary skill in the art to which the present invention pertains that the invention may be embodied in various modified forms without departing from the spirit or essential characteristics thereof. Thus, the embodiments described herein should be considered in an illustrative sense only and not for the purpose of limitation.

### INDUSTRIAL APPLICABILITY

A method of diagnosing chronic obstructive airway diseases through bacterial metagenomic analysis, according to the present invention, can be used to predict and diagnose a risk of developing chronic obstructive airway diseases such as asthma, COPD, and the like by analyzing an increase or decrease in content of extracellular vesicles derived from specific bacteria through bacterial metagenomic analysis using a subject-derived sample. Extracellular vesicles secreted from bacteria present in the environment are absorbed into the human body, and thus may directly affect the occurrence of inflammation, and it is difficult to diagnose chronic obstructive airway diseases such as asthma, COPD, and the like early before symptoms occur, and thus efficient treatment therefor is difficult. Thus, according to the present invention, a risk of developing chronic obstructive airway diseases such as asthma, COPD, and the like can be predicted through metagenomic analysis of bacteria or bacteria-derived extracellular vesicles by using a human body-derived sample, and thus the onset of chronic obstructive airway diseases can be delayed or prevented through appropriate management by early diagnosis and prediction of a risk group for chronic obstructive airway diseases, and, even after chronic obstructive airway diseases occur, early diagnosis therefor can be implemented, thereby lowering the incidence rate of chronic obstructive airway diseases and increasing therapeutic effects. In addition, patients diagnosed with asthma or COPD are able to avoid exposure to causative factors predicted by metagenomic analysis, whereby the progression of asthma and COPD can be ameliorated, or recurrence thereof can be prevented.

1. A method of providing information for the diagnosis of a chronic obstructive airway disease, the method comprising:
(a) extracting DNA from extracellular vesicles isolated from a subject sample;
(b) performing polymerase chain reaction (PCR) on the extracted DNA using a pair of primers having SEQ ID NO: 1 and SEQ ID NO: 2; and
(c) comparing an increase or decrease in content of bacteria-derived extracellular vesicles between a normal individual-derived sample and a chronic obstructive pulmonary disease (COPD) patient-derived sample through sequencing of a product of the PCR,
comparing an increase or decrease in content of bacteria-derived extracellular vesicles between a normal individual-derived sample and an asthma patient-derived sample through sequencing of a product of the PCR, or
comparing an increase or decrease in content of bacteria-derived extracellular vesicles between an asthma patient-derived sample and a COPD patient-derived sample through sequencing of a product of the PCR.

2. The method of claim 1, wherein, in process (c), COPD is diagnosed by comparing an increase or decrease in content of bacteria-derived extracellular vesicles between a normal individual-derived sample and a COPD patient-derived sample through sequencing of a product of the PCR.

3. The method of claim 2, wherein, in process (c), the comparing comprises comparing an increase or decrease in content of:
extracellular vesicles derived from bacteria belonging to the phylum Tenericutes;
extracellular vesicles derived from one or more bacteria selected from the group consisting of the class Mollicutes and the class Solibacteres;
extracellular vesicles derived from one or more bacteria selected from the group consisting of the order Stramenopiles, the order Rubrobacterales, the order Turicibacterales, the order Rhodocyclales, the order RF39, and the order Solibacterales;
extracellular vesicles derived from one or more bacteria selected from the group consisting of the family Rubrobacteraceae, the family Turicibacteraceae, the family Rhodocyclaceae, the family Nocardiaceae, the family Clostridiaceae, the family S24-7, the family Staphylococcaceae, and the family Gordoniaceae; or
extracellular vesicles derived from one or more bacteria selected from the group consisting of the genus Hydrogenophilus, the genus Proteus, the genus Geobacillus, the genus Chromohalobacter, the genus Rubrobacter, the genus Megamonas, the genus Turicibacter, the genus Rhodococcus, the genus Phascolarctobacterium, the genus SMB53, the genus Desulfovibrio, the genus Jeotgalicoccus, the genus Cloacibacterium, the genus Klebsiella, the genus Escherichia, the genus Cupriavidus, the genus Adlercreutzia, the genus Clostridium, the genus Faecalibacterium, the genus Stenotrophomonas, the genus Staphylococcus, the genus Gordonia, the genus Micrococcus, the genus Coprococcus, the genus Novosphingobium, the genus Enhydrobacter, the genus Citrobacter, and the genus Brevundimonas.

4. The method of claim 1, wherein, in process (c), asthma is diagnosed by comparing an increase or decrease in content of bacteria-derived extracellular vesicles between a normal individual-derived sample and an asthma patient-derived sample through sequencing of a product of the PCR.

5. The method of claim 4, wherein, in process (c), the comparing comprises comparing an increase or decrease in content of:
extracellular vesicles derived from one or more bacteria selected from the group consisting of the phylum Chlorollexi, the phylum Armatimonadetes, the phylum Fusobacteria, the phylum Cyanobacteria, the phylum Planctomycetes, the phylum Thermi, the phylum Verrucomicrobia, the phylum Acidobacteria, and the phylum TM7;
extracellular vesicles derived from one or more bacteria selected from the group consisting of the class Rubrobacteria, the class Fimbriimonadia, the class Cytophagia, the class Chloroplast, the class Fusobacteriia, the class Saprospirae, the class Sphingobacteriia, the class Deinococci, the class Verrucomicrobiae, the class TM7-3, the class Alphaproteobacteria, the class Flavobacteriia, the class Bacilli, and the class 4C0d-2;
extracellular vesicles derived from one or more bacteria selected from the group consisting of the order Rubrobacterales, the order Stramenopiles, the order Bacillales, the order Rhodocyclales, the order Fimbriimonadales, the order Cytophagales, the order Rickettsiales, the order Alteromonadales, the order Actinomycetales, the order Streptophyta, the order Fusobacteriales, the order CW040, the order Saprospirales, the order Aeromonadales, the order Neisseriales, the order Rhizobiales, the order Pseudomonadales, the order Deinococcales, the order Xanthomonadales, the order Sphingomonadales, the order Sphingobacteriales, the order Verrucomicrobiales, the order Flavobacteriales, the order Caulobacterales, the order Enterobacteriales, the order Bifidobacteriales, and the order YS2;
extracellular vesicles derived from one or more bacteria selected from the group consisting of the family Rubrobacteraceae, the family Exiguobacteraceae, the family Nocardiaceae, the family F16, the family Pseudonocardiaceae, the family Dermabacteraceae, the family Brevibacteriaceae, the family Microbacteriaceae, the family Staphylococcaceae, the family Cytophagaceae, the family Planococcaceae, the family Tissierellaceae, the family Rhodocyclaceae, the family Propionibacteriaceae, the family Fimbriimonadaceae, the family Campylobacteraceae, the family Dermacoccaceae, the family Burkholderiaceae, the family Rhizobiaceae, the family Bacillaceae, the family Corynebacteriaceae, the family mitochondria, the family Fusobacteriaceae, the family Leptotrichiaceae, the family Pseudomonadaceae, the family Bradyrhizobiaceae, the family Aeromonadaceae, the family Neisseriaceae, the family Methylobacteriaceae, the family Carnobacteriaceae, the family Xanthomonadaceae, the family Geodermatophilaceae, the family Mycobacteriaceae, the family Gordoniaceae, the family Micrococcaceae, the family Hyphomicrobiaceae, the family Moraxellaceae, the family Sphingomonadaceae, the family Actinomycetaceae, the family Deinococcaceae, the family Intrasporangiaceae, the family Flavobacteriaceae, the family Lactobacillaceae, the family Verrucomicrobiaceae, the family Nocardioidaceae, the family Sphingobacteriaceae, the family Rhodospirillaceae, the family Caulobacteraceae, the family Weeksellaceae, the family Dietziaceae, the family Aerococcaceae, the family Porphyromonadaceae, the family Veillonellaceae, the family Enterobacteriaceae, the family Barnesiellaceae, the family Rikenellaceae, the family Bacteroidaceae, and the family Bifidobacteriaceae; or
extracellular vesicles derived from one or more bacteria selected from the group consisting of the genus Geobacillus, the genus Rubrobacter, the genus Exiguobacterium, the genus Ralstonia, the genus Sporosarcina, the genus Hydrogenophilus, the genus Rhodococcus, the genus Proteus, the genus Leptotrichia, the genus Brevibacterium, the genus Brachybacterium, the genus Staphylococcus, the genus Peptomphilus, the genus Lautropia, the genus Finegoldia, the genus Anaerococcus, the genus Sphingobacterium, the genus Propionibacterium, the genus Micrococcus, the genus Fimbriimonas, the genus Dermacoccus, the genus Campylobacter, the genus Agrobacterium, the genus Neisseria, the genus Acinetobacter, the genus Thermus, the genus Corynebacterium, the genus Fusobacterium, the genus Pseudomonas, the genus Jeotgalicoccus, the genus Dietzia, the genus Rubellimicrobium, the genus Flavobacterium, the genus Megamonas, the genus Porphyromonas, the genus Granulicatella, the genus Novosphingobium, the genus Sphingomonas, the genus Mycobacterium, the genus Methylobacterium, the genus Gordonia, the genus Burkholderia, the genus Kocuria, the genus Lactobacillus, the genus Deinococcus, the genus Kaistobacter, the genus Akkermansia, the genus Actinomyces, the genus Brevundimonas, the genus Virgibacillus, the genus Bacillus, the genus Eubacterium, the genus Rothia, the genus Chryseobacterium, the genus Faecalibacterium, the genus Roseburia, the genus Klebsiella, the genus Sutterella, the genus Paraprevotella, the genus Parabacteroides, the genus Butyricimonas, the genus Lachnobacterium, the genus Veillonella, the genus Bacteroides, the genus Lachnospira, the genus Bifidobacterium, the genus Bilophila, and the genus Enterobacter.

6. The method of claim 1, wherein, in process (c), asthma and COPD are differentially diagnosed by comparing an increase or decrease in content of bacteria-derived extracellular vesicles between an asthma patient-derived sample and a COPD patient-derived sample through sequencing of a product of the PCR.

7. The method of claim 6, wherein, in process (c), the comparing comprises comparing an increase or decrease in content of:
extracellular vesicles derived from one or more bacteria selected from the group consisting of the phylum Bacteroidetes, the phylum Tenericutes, the phylum Thermi, the phylum TM7, the phylum Cyanobacteria, the phylum Verrucomicrobia, the phylum Fusobacteria, the phylum Acidobacteria, the phylum Planctomycetes, the phylum Armatimonadetes, and the phylum Chlorollexi;
extracellular vesicles derived from one or more bacteria selected from the group consisting of the class Bacteroidia, the class 4C0d-2, the class Mollicutes, the class Bacilli, the class Deinococci, the class TM7-3, the class Flavobacteriia, the class Alphaproteobacteria, the class Verrucomicrobiae, the class Fusobacteriia, the class Saprospirae, the class Sphingobacteriia, the class Chloroplast, the class Cytophagia, the class Fimbriimonadia, the class Thermomicrobia, and the class Solibacteres;
extracellular vesicles derived from one or more bacteria selected from the group consisting of the order YS2, the order Bifidobacteriales, the order Turicibacterales, the order Bacteroidales, the order RF39, the order Enterobacteriales, the order Rhodobacterales, the order Neisseriales, the order Gemellales, the order Deinococcales, the order Flavobacteriales, the order Xanthomonadales, the order Verrucomicrobiales, the order Sphingomonadales, the order Caulobacterales, the order Fusobacteriales, the order Saprospirales, the order Pseudomonadales, the order Sphingobacteriales, the order Rhizobiales, the order Actinomycetales, the order CW040, the order Streptophyta, the order Rickettsiales, the order Alteromonadales, the order Cytophagales, the order Aeromonadales, the order Fimbriimonadales, the order JG30-KF-CM45, the order Bacillales, and the order Solibacterales;
extracellular vesicles derived from one or more bacteria selected from the group consisting of the family Helicobacteraceae, the family Bacteroidaceae, the family Bifidobacteriaceae, the family Turicibacteraceae, the family Rikenellaceae, the family Odoribacteraceae, the family Clostridiaceae, the family Barnesiellaceae, the family Veillonellaceae, the family Porphyromonadaceae, the family Enterobacteriaceae, the family Christensenellaceae, the family Lactobacillaceae, the family Rhodobacteraceae, the family Nocardiaceae, the family Neisseriaceae, the family Gemellaceae, the family Carnobacteriaceae, the family Aerococcaceae, the family Weeksellaceae, the family Deinococcaceae, the family Leptotrichiaceae, the family Mycobacteriaceae, the family Dietziaceae, the family Xanthomonadaceae, the family Ps eudomonadaceae, the family Verrucomicrobiaceae, the family Methylobacteriaceae, the family Flavobacteriaceae, the family Actinomycetaceae, the family Burkholderiaceae, the family Nocardioidaceae, the family Caulobacteraceae, the family Sphingomonadaceae, the family Corynebacteriaceae, the family Tissierellaceae, the family Chitinophagaceae, the family mitochondria, the family Sphingobacteriaceae, the family Fusobacteriaceae, the family Moraxellaceae, the family Micrococcaceae, the family Geodermatophilaceae, the family Dermacoccaceae, the family Intrasporangiaceae, the family Dermabacteraceae, the family Propionibacteriaceae, the family Rhodospirillaceae, the family Bradyrhizobiaceae, the family Campylobacteraceae, the family Brevibacteriaceae, the family Microbacteriaceae, the family Cellulomonadaceae, the family Gordoniaceae, the family Bacillaceae, the family Planococcaceae, the family Rhizobiaceae, the family Aeromonadaceae, the family Fimbriimonadaceae, the family Cytophagaceae, the family F16, the family Staphylococcaceae, the family Exiguobacteraceae, and the family Alteromonadaceae; or
extracellular vesicles derived from one or more bacteria selected from the group consisting of the genus Enterobacter, the genus Trabulsiella, the genus Phascolarctobacterium, the genus Klebsiella, the genus Bifidobacterium, the genus Bacteroides, the genus Turicibacter, the genus Sutterella, the genus Butyricimonas, the genus Parabacteroides, the genus Ruminococcus, the genus Veillonella, the genus Pediococcus, the genus Desulfovibrio, the genus SMB53, the genus Roseburia, the genus Odoribacter, the genus Dialister, the genus Escherichia, the genus Sphingobium, the genus Rothia, the genus Paracoccus, the genus Lactobacillus, the genus Rhodococcus, the genus Eubacterium, the genus Granulicatella, the genus Kaistobacter, the genus Capnocytophaga, the genus Deinococcus, the genus Mycobacterium, the genus Microbispora, the genus Methylobacterium, the genus Chryseobacterium, the genus Actinomyces, the genus Porphyromonas, the genus Kocuria, the genus Akkermansia, the genus Pseudomonas, the genus Coprococcus, the genus Peptomphilus, the genus Neisseria, the genus Corynebacterium, the genus Anaerococcus, the genus Acinetobacter, the genus Rubellimicrobium, the genus Sphingobacterium, the genus Sphingomonas, the genus Pedobacter, the genus Finegoldia, the genus Fusobacterium, the genus Lautropia, the genus Moraxella, the genus Enhydrobacter, the genus Dermacoccus, the genus Thermus, the genus Citrobacter, the genus Bacillus, the genus Stenotrophomonas, the genus Hymenobacter, the genus Brachybacterium, the genus Propionibacterium, the genus Leptotrichia, the genus Dietzia, the genus Brevibacterium, the genus Flavobacterium, the genus Gordonia, the genus Agrobacterium, the genus Fimbriimonas, the genus Novosphingobium, the genus Lysinibacillus, the genus Brevundimonas, the genus Achromobacter, the genus Micrococcus, the genus Staphylococcus, the genus Ralstonia, the genus Exiguobacterium, and the genus Alkanindiges.

8. The method of claim 1, wherein the subject sample is blood.

9. The method of claim 8, wherein the blood is whole blood, serum, plasma, or blood mononuclear cells.

10. A method of diagnosing chronic obstructive airway disease, the method comprising:
(a) extracting DNA from extracellular vesicles isolated from a subject sample;
(b) performing polymerase chain reaction (PCR) on the extracted DNA using a pair of primers having SEQ ID NO: 1 and SEQ ID NO: 2; and
(c) comparing an increase or decrease in content of bacteria-derived extracellular vesicles between a normal individual-derived sample and a chronic obstructive pulmonary disease (COPD) patient-derived sample through sequencing of a product of the PCR,
comparing an increase or decrease in content of bacteria-derived extracellular vesicles between a normal individual-derived sample and an asthma patient-derived sample through sequencing of a product of the PCR, or
comparing an increase or decrease in content of bacteria-derived extracellular vesicles between an asthma patient-derived sample and a COPD patient-derived sample through sequencing of a product of the PCR.

11. The method of claim 10, wherein, in process (c), COPD is diagnosed by comparing an increase or decrease in content of bacteria-derived extracellular vesicles between a normal individual-derived sample and a COPD patient-derived sample through sequencing of a product of the PCR.

12. The method of claim 11, wherein, in process (c), the comparing comprises comparing an increase or decrease in content of:
extracellular vesicles derived from bacteria belonging to the phylum Tenericutes;
extracellular vesicles derived from one or more bacteria selected from the group consisting of the class Mollicutes and the class Solibacteres;
extracellular vesicles derived from one or more bacteria selected from the group consisting of the order Stramenopiles, the order Rubrobacterales, the order Turicibacterales, the order Rhodocyclales, the order RF39, and the order Solibacterales;
extracellular vesicles derived from one or more bacteria selected from the group consisting of the family Rubrobacteraceae, the family Turicibacteraceae, the family Rhodocyclaceae, the family Nocardiaceae, the family Clostridiaceae, the family S24-7, the family Staphylococcaceae, and the family Gordoniaceae; or
extracellular vesicles derived from one or more bacteria selected from the group consisting of the genus Hydrogenophilus, the genus Proteus, the genus Geobacillus, the genus Chromohalobacter, the genus Rubrobacter, the genus Megamonas, the genus Turicibacter, the genus Rhodococcus, the genus Phascolarctobacterium, the genus SMB53, the genus Desulfovibrio, the genus Jeotgalicoccus, the genus Cloacibacterium, the genus Klebsiella, the genus Escherichia, the genus Cupriavidus, the genus Adlercreutzia, the genus Clostridium, the genus Faecalibacterium, the genus Stenotrophomonas, the genus Staphylococcus, the genus Gordonia, the genus Micrococcus, the genus Coprococcus, the genus Novosphingobium, the genus Enhydrobacter, the genus Citrobacter, and the genus Brevundimonas.

13. The method of claim 10, wherein, in process (c), asthma is diagnosed by comparing an increase or decrease in content of bacteria-derived extracellular vesicles between a normal individual-derived sample and an asthma patient-derived sample through sequencing of a product of the PCR.

14. The method of claim 13, wherein, in process (c), the comparing comprises comparing an increase or decrease in content of:
extracellular vesicles derived from one or more bacteria selected from the group consisting of the phylum Chlorollexi, the phylum Armatimonadetes, the phylum Fusobacteria, the phylum Cyanobacteria, the phylum Planctomycetes, the phylum Thermi, the phylum Verrucomicrobia, the phylum Acidobacteria, and the phylum TM7;
extracellular vesicles derived from one or more bacteria selected from the group consisting of the class Rubrobacteria, the class Fimbriimonadia, the class Cytophagia, the class Chloroplast, the class Fusobacteriia, the class Saprospirae, the class Sphingobacteriia, the class Deinococci, the class Verrucomicrobiae, the class TM7-3, the class Alphaproteobacteria, the class Flavobacteriia, the class Bacilli, and the class 4C0d-2;
extracellular vesicles derived from one or more bacteria selected from the group consisting of the order Rubrobacterales, the order Stramenopiles, the order Bacillales, the order Rhodocyclales, the order Fimbriimonadales, the order Cytophagales, the order Rickettsiales, the order Alteromonadales, the order Actinomycetales, the order Streptophyta, the order Fusobacteriales, the order CW040, the order Saprospirales, the order Aeromonadales, the order Neisseriales, the order Rhizobiales, the order Pseudomonadales, the order Deinococcales, the order Xanthomonadales, the order Sphingomonadales, the order Sphingobacteriales, the order Verrucomicrobiales, the order Flavobacteriales, the order Caulobacterales, the order Enterobacteriales, the order Bifidobacteriales, and the order YS2;
extracellular vesicles derived from one or more bacteria selected from the group consisting of the family Rubrobacteraceae, the family Exiguobacteraceae, the family Nocardiaceae, the family F16, the family Pseudonocardiaceae, the family Dermabacteraceae, the family Brevibacteriaceae, the family Microbacteriaceae, the family Staphylococcaceae, the family Cytophagaceae, the family Planococcaceae, the family Tissierellaceae, the family Rhodocyclaceae, the family Propionibacteriaceae, the family Fimbriimonadaceae, the family Campylobacteraceae, the family Dermacoccaceae, the family Burkholderiaceae, the family Rhizobiaceae, the family Bacillaceae, the family Corynebacteriaceae, the family mitochondria, the family Fusobacteriaceae, the family Leptotrichiaceae, the family Pseudomonadaceae, the family Bradyrhizobiaceae, the family Aeromonadaceae, the family Neisseriaceae, the family Methylobacteriaceae, the family Carnobacteriaceae, the family Xanthomonadaceae, the family Geodermatophilaceae, the family Mycobacteriaceae, the family Gordoniaceae, the family Micrococcaceae, the family Hyphomicrobiaceae, the family Moraxellaceae, the family Sphingomonadaceae, the family Actinomycetaceae, the family Deinococcaceae, the family Intrasporangiaceae, the family Flavobacteriaceae, the family Lactobacillaceae, the family Verrucomicrobiaceae, the family Nocardioidaceae, the family Sphingobacteriaceae, the family Rhodospirillaceae, the family Caulobacteraceae, the family Weeksellaceae, the family Dietziaceae, the family Aerococcaceae, the family Porphyromonadaceae, the family Veillonellaceae, the family Enterobacteriaceae, the family Barnesiellaceae, the family Rikenellaceae, the family Bacteroidaceae, and the family Bifidobacteriaceae; or
extracellular vesicles derived from one or more bacteria selected from the group consisting of the genus Geobacillus, the genus Rubrobacter, the genus Exiguobacterium, the genus Ralstonia, the genus Sporosarcina, the genus Hydrogenophilus, the genus Rhodococcus, the genus Proteus, the genus Leptotrichia, the genus Brevibacterium, the genus Brachybacterium, the genus Staphylococcus, the genus Peptomphilus, the genus Lautropia, the genus Finegoldia, the genus Anaerococcus, the genus Sphingobacterium, the genus Propionibacterium, the genus Micrococcus, the genus Fimbriimonas, the genus Dermacoccus, the genus Campylobacter, the genus Agrobacterium, the genus Neisseria, the genus Acinetobacter, the genus Thermus, the genus Corynebacterium, the genus Fusobacterium, the genus Pseudomonas, the genus Jeotgalicoccus, the genus Dietzia, the genus Rubellimicrobium, the genus Flavobacterium, the genus Megamonas, the genus Porphyromonas, the genus Granulicatella, the genus Novosphingobium, the genus Sphingomonas, the genus Mycobacterium, the genus Methylobacterium, the genus Gordonia, the genus Burkholderia, the genus Kocuria, the genus Lactobacillus, the genus Deinococcus, the genus Kaistobacter, the genus Akkermansia, the genus Actinomyces, the genus Brevundimonas, the genus Virgibacillus, the genus Bacillus, the genus Eubacterium, the genus Rothia, the genus Chryseobacterium, the genus Faecalibacterium, the genus Roseburia, the genus Klebsiella, the genus Sutterella, the genus Paraprevotella, the genus Parabacteroides, the genus Butyricimonas, the genus Lachnobacterium, the genus Veillonella, the genus Bacteroides, the genus Lachnospira, the genus Bifidobacterium, the genus Bilophila, and the genus Enterobacter.

15. The method of claim 10, wherein, in process (c), asthma and COPD are differentially diagnosed by comparing an increase or decrease in content of bacteria-derived extracellular vesicles between an asthma patient-derived sample and a COPD patient-derived sample through sequencing of a product of the PCR.

16. The method of claim 15, wherein, in process (c), the comparing comprises comparing an increase or decrease in content of:
extracellular vesicles derived from one or more bacteria selected from the group consisting of the phylum Bacteroidetes, the phylum Tenericutes, the phylum Thermi, the phylum TM7, the phylum Cyanobacteria, the phylum Verrucomicrobia, the phylum Fusobacteria, the phylum Acidobacteria, the phylum Planctomycetes, the phylum Armatimonadetes, and the phylum Chlorollexi;
extracellular vesicles derived from one or more bacteria selected from the group consisting of the class Bacteroidia, the class 4C0d-2, the class Mollicutes, the class Bacilli, the class Deinococci, the class TM7-3, the class Flavobacteriia, the class Alphaproteobacteria, the class Verrucomicrobiae, the class Fusobacteriia, the class Saprospirae, the class Sphingobacteriia, the class Chloroplast, the class Cytophagia, the class Fimbriimonadia, the class Thermomicrobia, and the class Solibacteres;
extracellular vesicles derived from one or more bacteria selected from the group consisting of the order YS2, the order Bifidobacteriales, the order Turicibacterales, the order Bacteroidales, the order RF39, the order Enterobacteriales, the order Rhodobacterales, the order Neisseriales, the order Gemellales, the order Deinococcales, the order Flavobacteriales, the order Xanthomonadales, the order Verrucomicrobiales, the order Sphingomonadales, the order Caulobacterales, the order Fusobacteriales, the order Saprospirales, the order Pseudomonadales, the order Sphingobacteriales, the order Rhizobiales, the order Actinomycetales, the order CW040, the order Streptophyta, the order Rickettsiales, the order Alteromonadales, the order Cytophagales, the order Aeromonadales, the order Fimbriimonadales, the order JG30-KF-CM45, the order Bacillales, and the order Solibacterales;
extracellular vesicles derived from one or more bacteria selected from the group consisting of the family Helicobacteraceae, the family Bacteroidaceae, the family Bifidobacteriaceae, the family Turicibacteraceae, the family Rikenellaceae, the family Odoribacteraceae, the family Clostridiaceae, the family Barnesiellaceae, the family Veillonellaceae, the family Porphyromonadaceae, the family Enterobacteriaceae, the family Christensenellaceae, the family Lactobacillaceae, the family Rhodobacteraceae, the family Nocardiaceae, the family Neisseriaceae, the family Gemellaceae, the family Carnobacteriaceae, the family Aerococcaceae, the family Weeksellaceae, the family Deinococcaceae, the family Leptotrichiaceae, the family Mycobacteriaceae, the family Dietziaceae, the family Xanthomonadaceae, the family Pseudomonadaceae, the family Verrucomicrobiaceae, the family Methylobacteriaceae, the family Flavobacteriaceae, the family Actinomycetaceae, the family Burkholderiaceae, the family Nocardioidaceae, the family Caulobacteraceae, the family Sphingomonadaceae, the family Corynebacteriaceae, the family Tissierellaceae, the family Chitinophagaceae, the family mitochondria, the family Sphingobacteriaceae, the family Fusobacteriaceae, the family Moraxellaceae, the family Micrococcaceae, the family Geodermatophilaceae, the family Dermacoccaceae, the family Intrasporangiaceae, the family Dermabacteraceae, the family Propionibacteriaceae, the family Rhodospirillaceae, the family Bradyrhizobiaceae, the family Campylobacteraceae, the family Brevibacteriaceae, the family Microbacteriaceae, the family Cellulomonadaceae, the family Gordoniaceae, the family Bacillaceae, the family Planococcaceae, the family Rhizobiaceae, the family Aeromonadaceae, the family Fimbriimonadaceae, the family Cytophagaceae, the family F16, the family Staphylococcaceae, the family Exiguobacteraceae, and the family Alteromonadaceae; or
extracellular vesicles derived from one or more bacteria selected from the group consisting of the genus Enterobacter, the genus Trabulsiella, the genus Phascolarctobacterium, the genus Klebsiella, the genus Bifidobacterium, the genus Bacteroides, the genus Turicibacter, the genus Sutterella, the genus Butyricimonas, the genus Parabacteroides, the genus Ruminococcus, the genus Veillonella, the genus Pediococcus, the genus Desulfovibrio, the genus SMB53, the genus Roseburia, the genus Odoribacter, the genus Dialister, the genus Escherichia, the genus Sphingobium, the genus Rothia, the genus Paracoccus, the genus Lactobacillus, the genus Rhodococcus, the genus Eubacterium, the genus Granulicatella, the genus Kaistobacter, the genus Capnocytophaga, the genus Deinococcus, the genus Mycobacterium, the genus Microbispora, the genus Methylobacterium, the genus Chryseobacterium, the genus Actinomyces, the genus Porphyromonas, the genus Kocuria, the genus Akkermansia, the genus Pseudomonas, the genus Coprococcus, the genus Peptomphilus, the genus Neisseria, the genus Corynebacterium, the genus Anaerococcus, the genus Acinetobacter, the genus Rubellimicrobium, the genus Sphingobacterium, the genus Sphingomonas, the genus Pedobacter, the genus Finegoldia, the genus Fusobacterium, the genus Lautropia, the genus Moraxella, the genus Enhydrobacter, the genus Dermacoccus, the genus Thermus, the genus Citrobacter, the genus Bacillus, the genus Stenotrophomonas, the genus Hymenobacter, the genus Brachybacterium, the genus Propionibacterium, the genus Leptotrichia, the genus Dietzia, the genus Brevibacterium, the genus Flavobacterium, the genus Gordonia, the genus Agrobacterium, the genus Fimbriimonas, the genus Novosphingobium, the genus Lysinibacillus, the genus Brevundimonas, the genus Achromobacter, the genus Micrococcus, the genus Staphylococcus, the genus Ralstonia, the genus Exiguobacterium, and the genus Alkanindiges.

17. The method of claim 10, wherein the subject sample is blood.

18. The method of claim 17, wherein the blood is whole blood, serum, plasma, or blood mononuclear cells.


SEQUENCE LISTING (2 sequences)

SEQ ID NO: 1 - DNA, 50 bp, Artificial Sequence
  16S_V3_F
  misc_feature (42)..(42): any nucleotide
tcgtcggcagcgtcagatgtgtataagagacagcctacgggnggcwgcag

SEQ ID NO: 2 - DNA, 55 bp, Artificial Sequence
  16S_V4_R
gtctcgtgggctcggagatgtgtataagagacaggactachvgggtatctaatcc